(19) United States
(12) Patent Application Publication
(10) Pub. No.: US 2009/0208580 A1
(43) Pub. Date: Aug. 20, 2009

(54) Functionalized dendrimer-encapsulated and dendrimer-stabilized nanoparticles

(75) Inventors: Xiangyang Shi, Ann Arbor, MI (US); James R. Baker, JR., Ann Arbor, MI (US); Suhe Wang, Ann Arbor, MI (US)

Correspondence Address: Casimir Jones, S.C., 440 Science Drive, Suite 203, Madison, WI, 53711, US

(73) Assignee: Regents of the University of Michigan, Ann Arbor, MI (US)

(21) Appl. No.: 11/523,509

(22) Filed: Sep. 19, 2006

Related U.S. Application Data
(60) Provisional application No. 60/718,448, filed on Sep. 19, 2005.

(51) Int. Cl. A61K 9/14; B32B 1/00; A61K 31/7052; C12N 5/00; A61P 35/00; A61P 31/00

(52) U.S. Cl. 424/489; 428/402; 428/402.24; 514/44 R; 435/375

(57) ABSTRACT

The present invention relates to compositions comprising functionalized dendrimer-stabilized nanoparticles (DSNPs), functionalized dendrimer-encapsulated nanoparticles (DENPs) (e.g., metal DENPs), and methods of generating and using the same.

This application claims priority to U.S. Provisional Application No. 60/718,448, filed Sep. 19, 2005, the entire contents of which are incorporated herein by reference.

This invention was funded, in part, under National Cancer Institute (NCI), National Institute of Health (NIH) Contract N01-CO-27031-16, Contract NOI-CO-97111, Contract 1 RO1 CA119409, and Contract 1 RO1 EB002657, and U.S. Department of Energy Award No. FG01-00NE22943. The government may have certain rights in the invention.

### FIELD OF THE INVENTION

The present invention relates to compositions comprising functionalized dendrimer-stabilized nanoparticles (DSNPs), functionalized dendrimer-encapsulated nanoparticles (DENPs) (e.g., metal DENPs), and methods of generating and using the same.

### BACKGROUND OF THE INVENTION

Cancer is the second leading cause of death, resulting in one out of every four deaths, in the United States. In 1997, the estimated total number of new diagnoses for lung, breast, prostate, colorectal and ovarian cancer was approximately two million. Due to the ever increasing aging population in the United States, it is reasonable to expect that rates of cancer incidence will continue to grow.

Cancer is currently treated using a variety of modalities including surgery, radiation therapy and chemotherapy. The choice of treatment modality will depend upon the type, location and dissemination of the cancer. For example, many common neoplasms, such as colon cancer, respond poorly to available therapies.

For tumor types that are responsive to current methods, only a fraction of cancers respond well to the therapies. In addition, despite the improvements in therapy for many cancers, most currently used therapeutic agents have severe side effects. These side effects often limit the usefulness of chemotherapeutic agents and result in a significant portion of cancer patients without any therapeutic options. Other types of therapeutic initiatives, such as gene therapy or immunotherapy, may prove to be more specific and have fewer side effects than chemotherapy. However, while showing some progress in a few clinical trials, the practical use of these approaches remains limited at this time.

Despite the limited success of existing therapies, the understanding of the underlying biology of neoplastic cells has advanced. The cellular events involved in neoplastic transformation and altered cell growth are now identified and the multiple steps in carcinogenesis of several human tumors have been documented (See e.g., Isaacs, Cancer 70:1810 (1992)). Oncogenes that cause unregulated cell growth have been identified and characterized as to genetic origin and function. Specific pathways that regulate the cell replication cycle have been characterized in detail and the proteins involved in this regulation have been cloned and characterized. Also, molecules that mediate apoptosis and negatively regulate cell growth have been clarified in detail (Kerr et al., Cancer 73:2013 (1994)). It has now been demonstrated that manipulation of these cell regulatory pathways has been able to stop growth and induce apoptosis in neoplastic cells (See e.g., Cohen and Tohoku, Exp. Med., 168:351 (1992) and Fujiwara et al., J. Natl. Cancer Inst., 86:458 (1994)). The metabolic pathways that control cell growth and replication in neoplastic cells are important therapeutic targets.

Despite these impressive accomplishments, many obstacles still exist before these therapies can be used to treat cancer cells in vivo. For example, these therapies require the identification of specific pathophysiologic changes in an individual's particular tumor cells. This requires mechanical invasion (biopsy) of a tumor and diagnosis typically by in vitro cell culture and testing. The tumor phenotype then has to be analyzed before a therapy can be selected and implemented. Such steps are time consuming, complex, and expensive.

There is a need for treatment methods that are selective for tumor cells compared to normal cells. Current therapies are only relatively specific for tumor cells. Although tumor targeting addresses this selectivity issue, it is not adequate, as most tumors do not have unique antigens. Further, the therapy ideally should have several, different mechanisms of action that work in parallel to prevent the selection of resistant neoplasms. The therapy ideally should allow the physician to identify residual or minimal disease before and immediately after treatment, and to monitor the response to therapy. This is important since a few remaining cells may result in re-growth, or worse, lead to a tumor that is resistant to therapy. Identifying residual disease at the end of therapy (i.e., rather than after tumor regrowth) may facilitate eradication of the few remaining tumor cells.

Thus, an ideal therapy should have the ability to target a tumor, image the extent of the tumor (e.g., tumor metastasis) and identify the presence of the therapeutic agent in the tumor cells. Thus, therapies are needed that allows the physician to select therapeutic molecules based on the pathophysiologic abnormalities in the tumor cells, to document the response to the therapy, and to identify residual disease.

### SUMMARY OF THE INVENTION

The present invention relates to compositions comprising functionalized dendrimer-stabilized nanoparticles (DSNPs), functionalized dendrimer-encapsulated nanoparticles (DFNPs) (e.g., metal DENPs), and methods of generating and using the same

Accordingly, in some embodiments, the present invention provides a method of synthesizing a functionalized (e.g., acetamide-functionalized) dendrimer encapsulated nanoparticle (DENP) comprising: providing a DENP and reacting DENP with acetic anhydride. In some embodiments, the nanoparticle is a metal. In some embodiments, the metal is gold. In some embodiments, the metal includes, but is not limited to, copper, platinum, silver, lead, cobalt, iron, manganese, chromium and nickel. In some embodiments, the nanoparticle is a semiconductor quantum dot. The present invention is not limited by the type of quantum dot. Indeed, a variety of quantum dots are contemplated to be useful in the present invention including, but not limited to, CdSe, CdS, PbSe and PbS.

The present invention also provides a method of synthesizing a hydroxyl-functionalized DENP comprising: providing a DENP and reacting the DENP with glycidol. In some embodiments, the nanoparticle is a metal. In some embodiments, the metal is gold. In some embodiments, the metal includes, but is not limited to, copper, platinum, silver, lead, cobalt, iron, manganese, chromium and nickel.

The present invention also provides a method of synthesizing an acetamide-functionalized dendrimer stabilized nanoparticle comprising providing a solution comprising {(Au³⁺)₇-G5.NH₂} complex and triethylamine; and a solution of acetic anhydride; and mixing the two solutions. In some embodiments, the solution of acetic anhydride is a methanol-based solution. In some embodiments, the nanoparticle is a metal.

The present invention also provides a method of synthesizing a hydroxyl-functionalized dendrimer stabilized nanoparticle comprising providing a solution comprising G5.NGlyOH dendrimenrs and a solution comprising HAuCl₄; and mixing the two solutions. In some embodiments, each of the solutions are methanol-based solutions. In some embodiments, the nanoparticle is a metal.

The present invention also provides a composition comprising a functionalized (e.g., an acetamide- or hydroxyl-functionalized) DENP and/or a functionalized dendrimer stabilized nanoparticle (DSNP). In some embodiments, the functionalized DENP is <5 nm in size (e.g., is between 1-5 nm, between 2-4 nm, between 2-3 nm, or between 2-5 nm in size, although smaller (e.g., <1 nm) DENPs are also contemplated). In some embodiments, a DENP is larger than 5 nm in size. In some embodiments, the functionalized DSNP is <20 nm in size (e.g., is between 5-15 nm or between 10-15 rn in size). In some embodiments, a DSNP may be larger than 20 nm. In some embodiments, the DENP comprises one dendrimer per one metal nanoparticle. In some embodiments, the DENP comprises one dendrimer encapsulating multiple nanoparticles. In some embodiments, a DSNP comprises one nanoparticle stabilized by multiple dendrimers. In some embodiments, the nanoparticle is a metal. In some embodiments, the metal is gold. In some embodiments, the metal includes, but is not limited to, copper, platinum, silver, lead, cobalt, iron, manganese, chromium and nickel. In some embodiments, the functionalized DENP and/or DSNP comprises one or more functional groups. In some embodiments, the functional groups comprises a therapeutic agent, a targeting agent, an imaging agent, and/or a biological monitoring agent. In some embodiments, the therapeutic agent comprises a chemotherapeutic compound (e.g., methotrexate). In some embodiments of the present invention, the therapeutic agent comprises, but is not limited to, a chemotherapeutic agent, an anti-oncogenic agent, an anti-vascularizing agent, a tumor suppressor agent, an anti-microbial agent, or an expression construct comprising a nucleic acid encoding a therapeutic protein, although the present invention is not limited by the nature of the therapeutic agent. In further embodiments, the therapeutic agent is protected with a protecting group selected from photo-labile, radio-labile, and enzyme-labile protecting groups. In some embodiments, the chemotherapeutic agent is selected from a group comprising, but not limited to, platinum complex, verapamil, podophylltoxin, carboplatin, procarbazine, mechloroethamine, cyclophosphamide, camptothecin, ifosfamide, melphalan, chlorambucil, bisulfan, nitrosurea, adriamycin, dactinomycin, daunorubicin, doxorubicin, bleomycin, plicomycin, mitomycin, bleomycin, etoposide, tamoxifen, paclitaxel, taxol, transplatinum, 5-fluorouracil, vincristin, vinblastin, and methotrexate. In some embodiments, the anti-oncogenic agent comprises an antisense nucleic acid (e.g., RNA, molecule). In certain embodiments, the antisense nucleic acid comprises a sequence complementary to an RNA of an oncogene. In preferred embodiments, the oncogene includes, but is not limited to, abl, Bcl-2, Bcl-xL, erb, fms, gsp, hst, jun, myc, neu, raf; ras, ret, src, or trk. In some embodiments, the nucleic acid encoding a therapeutic protein encodes a factor including, but not limited to, a tumor suppressor, cytokine, receptor, inducer of apoptosis, or differentiating agent. In preferred embodiments, the tumor suppressor includes, but is not limited to, BRCA1, BRCA2, C-CAM, p16, p21, p53, p73, Rb, and p27. In preferred embodiments, the cytokine includes, but is not limited to, GMCSF, IL-1, IL-2, IL-3, IL-4, IL-5, IL-6, IL-7, IL-8, IL-9, IL-10, IL-11, IL-12, IL-13, IL-14, IL-15, β-interferon, γ-interferon, and TNF. In preferred embodiments, the receptor includes, but is not limited to, CFTR, EGFR, estrogen receptor, IL-2 receptor, and VEGFR. In preferred embodiments, the inducer of apoptosis includes, but is not limited to, AdE1B, Bad, Bak, Bax, Bid, Bik, Bim, Harakid, and ICE-CED3 protease. In some embodiments, the therapeutic agent comprises a short-half life radioisotope.

The present invention is not limited by type of anti-oncogenic agent or chemotherapeutic agent used (e.g., conjugated to a dendrimer of the present invention). Indeed, a variety of anti-oncogenic agents and chemotherapeutic agents are contemplated to be useful in the present invention including, but not limited to, Acivicin; Aclarubicin; Acodazole Hydrochloride; Acronine; Adozelesin; Adriamycin; Aldesleukin; Alitretinoin; Allopurinol Sodium; Altretamine; Ambomycin; Ametantrone Acetate; Aminoglutethimide; Amsacrine; Anastrozole; Annonaceous Acetogenins; Anthramycin; Asimicin; Asparaginase; Asperlin; Azacitidine; Azetepa; Azotomycin; Batimastat; Benzodepa; Bexarotene; Bicalutamide; Bisantrene Hydrochloride; Bisnafide Dimesylate; Bizelesin; Bleomycin Sulfate; Brequinar Sodium; Bropirimine; Bullatacin; Busulfan; Cabergoline; Cactinomycin; Calusterone; Caracemide; Carbetimer; Carboplatin; Carmustine; Carabicin Hydrochloride; Carzelesin; Cedefingol; Celecoxib; Chlorambucil; Cirolemycin; Cisplatin; Cladribine; Crisnatol Mesylate; Cyclophosphamide; Cytarabine; Dacarbazine; DACA (N-[2-(Dimethyl-amino)ethyl]acridine-4-carboxamide); Dactinomycin; Daunorubicin Hydrochloride; Daunomycin; Decitabine; Denileukin Diftitox; Dexormaplatin; Dezaguanine; Dezaguanine Mesylate; Diaziquone; Docetaxel; Doxorubicin; Doxorubicin Hydrochloride; Droloxifene; Droloxifene Citrate; Dromostanolone Propionate; Duazomycin; Edatrexate; Eflomithine Hydrochloride; Elsamitrucin; Enloplatin; Enpromate; Epipropidine; Epirubicin Hydrochloride; Erbulozole; Esorubicin Hydrochloride; Estramustine; Estramustine Phosphate Sodium; Etanidazole; Ethiodized Oil I 131; Etoposide; Etoposide Phosphate; Etoprine; Fadrozole Hydrochloride; Fazarabine; Fenretinide; Floxuridine; Fludarabine Phosphate; Fluorouracil; 5-FdUMP; Fluorocitabine; Fosquidone; Fostriecin Sodium; FK-317; FK-973; FR-66979; FR-900482; Gemcitabine; Geimcitabine Hydrochloride; Gemtuzumab Ozogamicin; Gold Au 198; Goserelin Acetate; Guanacone; Hydroxyurea; Idarubicin Hydrochloride; Ifosfamide; Ilmofosine; Interferon Alfa-2a; Interferon Alfa-2b; Interferon Alfa-n 1; Interferon Alfa-n3; Interferon Beta-1a; Interferon Gamma-1b; Iproplatin; Irinotecan Hydrochloride; Lanreotide Acetate; Letrozole; Leuprolide Acetate; Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Methoxsalen; Metoprine; Meturedepa; Mitindomide; Mitocarcin; Mitocromin; Mitogillin; Mitomalcin; Mitomycin; Mytomycin C; Mitosper; Mitotane; Mitoxantrone Hydrochloride; Mycophenolic Acid; Nocodazole; Nogalamycin; Oprelvekin; Ormaplatin; Oxisuran; Paclitaxel; Pamidronate Disodium; Pegaspargase; Peliomycin; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Riboprine; Rituximab; Rogletimide; Rolliniastatin; Safingol; Safingol Hydrochloride; Samarium/Lexidronam; Semustine; Simtrazene; Sparfosate Sodium; Sparsomycin; Spirogermanium Hydrochloride; Spiromustine; Spiroplatin; Squamocin; Squamotacin; Streptonigrin; Streptozocin; Strontium Chloride Sr 89; Sulofenur; Talisomycin; Taxane; Taxoid; Tecogalan Sodium; Tegafur; Teloxantrone Hydrochloride; Temoporfin; Teniposide; Teroxirone; Testolactone; Thiamiprine; Thioguanine; Thiotepa; Thymitaq; Tiazofurin; Tirapazamine; Tomudex; TOP-53; Topotecan Hydrochloride; Toremifene Citrate; Trastuzumab; Trestolone Acetate; Triciribine Phosphate; Trimetrexate; Trimetrexate Glucuronate; Triptorelin; Tubulozole Hydrochloride; Uracil Mustard; Uredepa; Valrubicin; Vapreotide; Verteporfin; Vinblastine; Vinblastine Sulfate; Vincristine; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; Zorubicin Hydrochloride; 2-Chlorodeoxyadenosine; 2′-Deoxyformycin; 9-aminocamptothecin; raltitrexed; N-propargyl-5,8-dideazafolic acid; 2-chloro-2′-arabino-fluoro-2′-deoxyadenosine; 2-chloro-2′-deoxyadenosine; anisomycin; trichostatin A; hPRL-G129R; CEP-751; linomide; sulfur mustard; nitrogen mustard (mechlorethamine); cyclophosphamide; melphalan; chlorambucil; ifosfamide; busulfan; N-methyl-N-nitrosourea (MNU); N,N′-Bis(2-chloroethyl)-N-nitrosourea (BCNU); N-(2-chloroethyl)-N′-cyclohex-yl-N-nitrosourea (CCNU); N-(2-chloroethyl)-N′-(trans-4-methylcyclohexyl-N-nitrosourea (MeCCNU); N-(2-chloroethyl)-N′-(diethyl)ethylphosphonate-N-nit-rosourea (fotemustine); streptozotocin; diacarbazine (DTIC); mitozolomide; temozolomide; thiotepa; mitomycin C; AZQ; adozelesin; Cisplatin; Carboplatin; Ormaplatin; Oxaliplatin; C1-973; DWA 2114R; JM216; JM335; Bis (platinum); tomudex; azacitidine; cytarabine; gemcitabine; 6-Mercaptopurine; 6-Thioguanine; Hypoxanthine; teniposide; 9-amino camptothecin; Topotecan; CPT-11; Doxorubicin; Daunomycin; Epirubicin; darubicin; mitoxantrone; losoxantrone; Dactinomycin (Actinomycin D); amsacrine; pyrazoloacridine; all-trans retinol; 14-hydroxy-retro-retinol; all-trans retinoic acid; N-(4-Hydroxyphenyl) retinamide; 13-cis retinoic acid; 3-Methyl TTNTEB; 9-cis retinoic acid; fludarabine (2-F-ara-AMP); and 2-chlorodeoxyadenosine (2-Cda).

Other anti-oncogenic agents and chemotherapeutic agents include antiproliferative agents (e.g., Piritrexim Isothionate), antiprostatic hypertrophy agents (e.g., Sitogluside), benign prostatic hyperplasia therapy agents (e.g., Tamsulosin Hydrochloride), prostate growth inhibitor agents (e.g., pentomone), and radioactive agents.

Yet other anti-oncogenic agents and chemotherapeutic agents may comprise anti-cancer supplementary potentiating agents, including tricyclic anti-depressant drugs (e.g., imipramine, desipramine, amitryptyline, clomipramine, trimipramine, doxepin, nortriptyline, protriptyline, amoxapine and maprotiline); non-tricyclic anti-depressant drugs (e.g., sertraline, trazodone and citalopram); Ca⁺⁺ antagonists (e.g., verapamil, nifedipine, nitrendipine and caroverine); Calmodulin inhibitors (e.g., prenylamine, trifluoroperazine and clomipramine); Amphotericin B; Triparanol analogues (e.g., tamoxifen); antiarrhythmic drugs (e.g., quinidine); antihypertensive drugs (e.g., reserpine); thiol depleters (e.g., buthionine and sulfoximine) and multiple drug resistance reducing agents such as Cremaphor EL.

Still other anti-oncogenic agents and chemotherapeutic agents are those selected from the group comprising annonaceous acetogenins; asimicin; rolliniastatin; guanacone, squamocin, bullatacin; squamotacin; taxanes; paclitaxel; gemcitabine; methotrexate FR-900482; FK-973; FR-66979; FK-317; 5-FU; FUDR; FdUMP; hydroxyurea; docetaxel; discodermolide; epothilones; vincristine; vinblastine; vinorelbine; meta-pac; irinotecan; SN-38; 10-OH campto; topotecan; etoposide; adriamycin; flavopiridol; Cis-Pt; carbo-Pt; bleomycin; mitomycin C; mithramycin; capecitabine; cytarabine; 2-C1-2′deoxyadenosine; Fludarabine-PO.sub.4; mitoxantrone; mitozolomide; pentostatin; and tomudex.

Yet other anti-oncogenic agents and chemotherapeutic agents comprise taxanes (e.g., paclitaxel and docetaxel). In some embodiments, the anti-oncogenic agent or chemotherapeutic agent comprises tamoxifen or the aromatase inhibitor arimidex (e.g., anastrozole).

In some embodiments of the present invention, the biological monitoring agent comprises an agent that measures an effect of a therapeutic agent (e.g., directly or indirectly measures a cellular factor or reaction induced by a therapeutic agent), however, the present invention is not limited by the nature of the biological monitoring agent. In some embodiments, the monitoring agent is capable of measuring the amount of or detecting apoptosis caused by the therapeutic agent.

In some embodiments of the present invention, the imaging agent comprises a radioactive label including, but not limited to ¹⁴C, ³⁶Cl, ⁵⁷Co, ⁵⁸Co, ⁵¹Cr, ¹²⁵I, ¹³¹I, ¹¹¹Ln, ¹⁵²Eu, ⁵⁹Fe, ⁶⁷Ga, ³²P, ¹⁸⁶Re, ³⁵S, ⁷⁵Se, Tc-99m, and ¹⁷⁵Yb. In some embodiments, the imaging agent comprises a fluorescing entity. In a preferred embodiment, the imaging agent is fluorescein isothiocyanate or 6-TAMRA. In some embodiments, the imaging agent is AlexFluo.

In some embodiments of the present invention, the targeting agent includes, but is not limited to an antibody, receptor ligand, hormone, vitamin, or antigen, however, the present invention is not limited by the nature of the targeting agent. In some embodiments, the antibody is specific for a disease-specific antigen. In some preferred embodiments, the disease-specific antigen comprises a tumor-specific antigen. In some embodiments, the receptor ligand includes, but is not limited to, a ligand for CFTR, EGFR, estrogen receptor, FGR2, folate receptor, IL-2 receptor, glycoprotein, and VEGFR. In a preferred embodiment, the receptor ligand is folic acid. Other embodiments that may be used with the present invention are described in U.S. Pat. No. 6,471,968 and WO 01/87348, each of which is herein incorporated by reference in their entireties.

In some embodiments, the functionalized DENP and/or DSNP is conjugated to the functional groups. In some embodiments, the conjugation comprises covalent bonds, ionic bonds, metallic bonds, hydrogen bonds, Van der Waals bonds, ester bonds or amide bonds. In some embodiments, the therapeutic agent comprises a chemotherapeutic agent, an anti-oncogenic agent, an anti-vascularizing agent, a tumor suppressor agent, an anti-microbial agent, or an expression construct comprising a nucleic acid encoding a therapeutic protein. In some embodiments, the therapeutic agent is protected with a protecting group. In some embodiments, the protecting group is selected from the group consisting of photo-labile protecting group, a radio-labile protecting group, and an enzyme-labile protecting group.

The present invention also provides a kit comprising a functionalized DENP and/or DSNP.

In some embodiments, any one of the functional groups (e.g., therapeutic agent) is provided in multiple copies on a single DENP and/or DSNP. Thus, in some embodiments, a single DENP and/or DSNP comprises 2-100 copies of a single functional group (e.g., a therapeutic agent such as methotrexate). In some embodiments, a DENP and/or DSNP comprises 2-5, 5-10, 10-20 or 20-50 copies of a single functional group. In some embodiments, a DENP and/or DSNP comprises 5-20 copies. In some embodiments, a DENP and/or DSNP comprises 50-100 or 100-200 copies of a functional group (e.g., a therapeutic agent, a targeting agent, or an imaging agent). In some embodiments, a DENP and/or DSNP comprises greater than 200 copies of a functional group. The invention further provides a DENP and/or DSNP that comprises multiple copies of two or more different functional group. For example, in some embodiments, the present invention provides a DENP and/or DSNP that comprises multiple copies (e.g., 2-10, 5-10, 10-15, 15-50, 50-100, 100-200, or more than 200 copies) of one type of functional group (e.g., a therapeutic agent such as methotrexate or any one of the other targeting agents discussed herein) and multiple copies (e.g., 2-10, 15-50, 50-100, 100-200, or more than 200 copies) of a second type of functional group (e.g., a targeting agent such as folic acid or any one of the other targeting agents discussed herein). In some embodiments, a DENP and/or DSNP comprises multiple copies of 2-10 different functional groups.

The present invention also provides a method of treating a disease (e.g., cancer or infectious disease) comprising administering to a subject suffering from or susceptible to the disease a therapeutically effective amount of a composition comprising functionalized DENP and/or DSNP of the present invention. In preferred embodiments, the DENPs and/or DSNPs of the present invention are configured such that they are readily cleared from the subject (e.g., so that there is little to no detectable toxicity at efficacious doses). In some embodiments, the disease is a neoplastic disease, for example, but not limited to, leukemia, acute leukemia, acute lymphocytic leukemia, acute myelocytic leukemia, myeloblastic, promyelocytic, myelomonocytic, monocytic, erythroleukemia, chronic leukemia, chronic myelocytic, (granulocytic) leukemia, chronic lymphocytic leukemia, Polycythemia vera, lymphoma, Hodgkin's disease, non-Hodgkin's disease, Multiple myeloma, Waldenstrom's macroglobulinemia, Heavy chain disease, solid tumors, sarcomas and carcinomas, fibrosarcoma, myxosarcoma, liposarcoma, chondrosarcoma, osteogenic sarcoma, chordoma, angiosarcoma, endotheliosarcoma, lymphangiosarcoma, lymphangioendotbeliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, uterine cancer, testicular tumor, lung carcinoma, small cell lung carcinoma, bladder carcinoma, epithelial carcinoma, glioma, astrocytoma, medulloblastoma, craniopharyngioma, ependymoma, pincalonma, hemangioblastoma, acoustic neuroma, oligodendroglioma, meningioma, melanoma, and neuroblastomaretinoblastoma. In some embodiments, the disease is an inflammatory disease selected from the group consisting of, but not limited to, eczema, inflammatory bowel disease, rheumatoid arthritis, asthma, psoriasis, ischemia/reperfusion injury, ulcerative colitis and acute respiratory distress syndrome. In some embodiments, the disease is a viral disease selected from the group consisting of, but not limited to, viral disease caused by hepatitis B, hepatitis C, rotavirus, human immunodeficiency virus type I (HIV-I), human immunodeficiency virus type II (HIV-II), human T-cell lymphotropic virus type I (HTLV-I), human T-cell lymphotropic virus type II (HTLV-II), AIDS, DNA viruses such as hepatitis type B and hepatitis type C virus; parvoviruses, such as adeno-associated virus and cytomegalovirus; papovaviruses such as papilloma virus, polyoma viruses, and SV40; adenoviruses; herpes viruses such as herpes simplex type I (HSV-I), herpes simplex type II (HSV-II), and Epstein-Barr virus; poxviruses, such as variola (smallpox) and vaccinia virus; and RNA viruses, such as human immunodeficiency virus type I (HIV-I), human immunodeficiency virus type II (HIV-II), human T-cell lymphotropic virus type I (HTLV-I), human T-cell lymphotropic virus type II (HTLV-II), influenza virus, measles virus, rabies virus, Sendai virus, picornaviruses such as poliomyelitis virus, coxsackieviruses, rhinoviruses, reoviruses, togaviruses such as rubella virus (German measles) and Semliki forest virus, arboviruses, and hepatitis type A virus.

The present invention also provides a method of treating a disease comprising administering to a subject suffering from or susceptible to the disease a therapeutically effective amount of a composition comprising a functionalized DENP and/or DSNP, the functionalized DENP and/or DSNP further comprising one or more functional groups, the one or more functional groups selected from the group consisting of a therapeutic agent, a targeting agent, and an imaging agent. In some embodiments, the disease is a neoplastic disease. In some embodiments, the neoplastic disease is selected from the group comprising leukemia, acute leukemia, acute lymphocytic leukemia, acute myelocytic leukemia, myeloblastic, promyelocytic, myelomonocytic, monocytic, erythroleukemia, chronic leukemia, chronic myelocytic, (granulocytic) leukemia, chronic lymphocytic leukemia, Polycythemia vera, lymphoma, Hodgkin's disease, non-Hodgkin's disease, Multiple myeloma, Waldenstrom's macroglobulinemia, Heavy chain disease, solid tumors, sarcomas and carcinomas, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, uterine cancer, testicular tumor, lung carcinoma, small cell lung carcinoma, bladder carcinoma, epithelial carcinoma, glioma, astrocytoma, medulloblastoma, craniopharyngioma, ependymoma, pincaloma, hemangioblastoma, acoustic neuroma, oligodendroglioma, meningioma, melanoma, and neuroblastomaretinoblastoma.

The present invention also provides a method of altering tumor growth in a subject, comprising providing a composition comprising functionalized DENP and/or DSNP, the DENP and/or DSNP further comprising one or more functional groups, the one or more functional groups selected from the group consisting of a therapeutic agent, a targeting agent, and an imaging agent; and administering the composition to the subject under conditions such that the tumor growth is altered. In some embodiments, altering comprises inhibiting tumor growth in the subject. In some embodiments, altering comprises reducing the size of the tumor in the subject. In some embodiments, the composition comprising a functionalized DENP and/or DSNP is co-administered with a chemotherapeutic agent or anti-oncogenic agent. In some embodiments, altering tumor growth sensitizes the tumor to chemotherapeutic or anti-oncogenic treatment.

The present invention also provides a method of targeting a functionalized DENP and/or DSNP to a cell comprising providing a functionalized dendrimer stabilized nanoparticle, wherein the functionalized DENP and/or DSNP comprises a cell specific targeting moiety; and a cell; and exposing the cell to the functionalized DENP and/or DSNP under conditions such that the targeting moiety interacts with the cell. In some embodiments, the DENP and/or DSNP is internalized by the targeted cell but not internalized by a non-targeted cell. In some embodiments, the targeted cell is a cancer cell. In some embodiments, the cell specific targeting moiety comprises folic acid. In some embodiments, the DENP and/or DSNP comprises one or more functional groups.

### DESCRIPTION OF THE DRAWINGS

FIG. 1 shows (a) UV-Vis and (b) fluorescence spectra of Au DSNPs. 1. {(Au⁰)₆-E2.NH₂}; 2. {(Au⁰)₁₂-E3.NH₂}; 3. {(Au⁰)₂₄-E4.NH₂}4. {(Au⁰)₅₇-E5.NH₂}; and 5. {(Au⁰)₉₈-E6.NH₂}. In (b), 6 and 7 indicate gold colloids with diameter of 5 and 100 min, respectively.

FIG. 2 shows large scale TEM images of (a) {(Au⁰)₆-E2.NH₂}; (b) {(Au⁰)₁₂—-E3.NH₂}; (c) {(Au⁰)₂₄-E4.NH₂}; (d) {(Au⁰)₅₇-E5.NH₂}; and (e) {(Au⁰)₉₈-E6.NH₂}; and their sizes as a function of the number of dendrimer generations (f).

FIG. 3 shows high-resolution TEM images of (a) {(Au⁰)₁₂-E3.NH₂} and (b) {(Au⁰)₅₇-E5.NH₂} DSNPs, (c) a typical SAED pattern of {(Au⁰)₉₈-E6.NH₂} DSNPs, and (d) an EDS spectrum of {(Au⁰)₂₄-E4.NH₂} DSNPs.

FIG. 4 shows PAGE electropherograms of Au DSNPs and their corresponding dendrimer stabilizers.

FIG. 5 shows a typical capillary electropherogram of (a) {(Au⁰)₅₇-E5.NH₂} DSNPs and UV-Vis spectra of (b) Peak 1, (c) Peak 2, (d) Peak 3, and (e) Peak 4 analyzed using Agilent CE software. Peak 1 indicates the internal standard 2,3-DAP. Peak 2, 3, 4 are related to the {(Au⁰)₅₇-E5.NH₂} DSNP species.

FIG. 6 depicts one embodiment of the present invention with reactions to modifiy Au DENPs prepared using amine-terminated E5.NH₂ dendrimers.

FIG. 7 depicts UV-Vis spectra of synthesized and modified Au DENPs.

FIG. 8 shows TEM micrographs of the (a) {(Au⁰)_{51.2}-E5.NH₂}, (c) {(Au⁰)_{51.2}-E5.NHAc}, and (e) {(Au⁰)_{51.2}-E5.NGlyOH} DENPs. The insets of (a), (c), and (e) show the high-resolution TEM images of respective individual DENPs. FIGS. 8 (b), (d) and (f) are size distribution histograms of the {(Au⁰)_{51.2}-E5.NH₂}, {(Au⁰)_{51.2}-E5.NHAc}, and {(Au⁰)_{51.2}-E5.NGlyOH} DENPs, respectively.

FIG. 9 shows PAGE electropherograms of Au DENPs and the corresponding dendrimers. Lane 1: E5.NH₂; Lane 2: E5.NHAc; Lane 3: E5.NGlyOH; Lane 4: {(Au⁰)_{51.2}-E5.NH₂}; Lane 5: {(Au⁰)_{51.2}-E5.NHAc}; and Lane 6: {(Au⁰)_{51.2}-E5.NGlyOH}.

FIG. 10 depicts a synthesis scheme of Au DENPs in one embodiment of the present invention.

FIG. 11 shows ¹H NMR and ¹³C NMR spectra of {(Au⁰)_{51.2}-E5.NH₂} Au DENPs and the corresponding dendrimer derivatives.

FIG. 12 shows ¹H NMR and ¹³C NMR spectra of {(Au⁰)_{51.2}-E5.NHAc} Au DENPs and the corresponding dendrimer derivatives.

FIG. 13 shows ¹H NMR and ¹³C NMR spectra of {(Au⁰)_{51.2}-E5.NGlyOH} Au DENPs and the corresponding dendrimer derivatives.

FIG. 14 shows UV-vis spectra of E5.NH₂, E5.NHAc, and E5.NGlyOH dendrimer derivatives.

FIG. 15 shows a photograph of the aqueous solutions of {(Au⁰)_{51.2}-E5.NH₂}, {(Au⁰)_{51.2}-E5.NHAc}, and {(Au⁰)_{51.2}-E5.NGlyOH} DENPs (from left to right).

FIG. 16 shows an MTT assay of KB cell viability after treatment with {(Au⁰)_{51.2}-E5.NH₂}, {(Au⁰)_{51.2}-E5.NHAc}, and {(Au⁰)_{51.2}-E5.NGlyOH} DENPs for 24 hours.

FIG. 17 shows phase-contrast photomicrographs of control KB cells without treatment with Au DENPs (a), KB cells treated with 2.0 μM {(Au⁰)_{51.2}-E5.NH₂} DENPs (b), KB cells treated with 2.0 μM {(Au⁰)_{51.2}-E5.NHAc} DENPs (c), and KB cells treated with 2.0 μM {(Au⁰)_{51.2}-E5.NGlyOH} DENPs (d).

FIG. 18 shows an MTT assay of KB cell viability after treatment with E5.NH₂, E5.NHAc, and E5.NGlyOH dendrimers for 24 hours.

FIG. 19 shows a photograph of the aqueous solutions of Au NPs synthesized using preformed E5.NHAc (1) and E5.NGlyOH (2) dendrimers as templates.

FIG. 20 shows a schematic representation of reactions involved in modifying Au DENPs for cancer cell targeting and imaging.

FIG. 21 shows ¹H NMR spectra of {(Au⁰)_{51.2}-G5-FI₅-Ac} (a) and {(Au⁰)_{51.2}-G5-FI₅-FA₅-Ac} (b) DENPs.

FIG. 22 shows characterization and toxicity test of functionalized Au DENPs. a and c; TEM images of the functionalized {(Au⁰)_{51.2}-G5-FI₅-Ac} and {(Au⁰)_{51.2}-G5-FI₅-FA₅-Ac} DENPs, respectively. b and d; The size distribution histograms of {(Au⁰)_{51.2}-G5-FI₅-Ac} and {(Au⁰)_{51.2}-G5-FI₅-FA₅-Ac} DENPs, respectively. e; UV-vis spectra of the starting {(Au⁰)_{51.2}-G5.NH₂} and functionalized {(Au⁰)_{51.2}-G5-FI₅-Ac} and {(Au⁰)_{51.2}-G5-FI₅-FA₅-Ac} DENPs. f; An MTT assay of KB cell viability after treatment with {(Au⁰)_{51.2}-G5-FI₅-Ac} and {(Au⁰)_{51.2}-G5-FI₅-FA₅-Ac} DENPs for 24 h.

FIG. 23 shows a photograph of the aqueous solutions of {(Au⁰)_{51.2}-G5-FI₅-Ac}(1) and {(Au⁰)_{51.2}-G5-FI₅—FA₅-Ac} (2) DENPs after 9 months of storage.

FIG. 24 shows flow cytometric and confocal microscopic studies of the binding of functionalized Au DENPs with KB cells. a and b, Binding of {(Au⁰)_{51.2}-G5-F₅-Ac} and {(Au⁰)_{51.2}-G5-FI₅-FA₅-Ac} DENPs (25 nM) with KB cells with high- and low-levels of FAR, respectively. 1. PBS control; 2. {(Au⁰)_{51.2}-G5-FI₅-Ac}; 3. {(Au⁰)_{51.2}-G5-FI₅-FA₅-Ac}. c and d, Dose-dependent binding of {(Au⁰)_{51.2}-G5-FI₅-Ac} and {(Au⁰)_{51.2}-G5-FI₅FA₅-Ac} DENPs with KB cells expressing high- and low-levels of FAR, respectively. e-g, Confocal microscopic images of KB cells with high-level FAR treated with PBS buffer (e), {(Au⁰)_{51.2}-G5-FI₅-Ac} (25 nM) (f), and {(Au⁰)_{51.2}-G5-FI₅-FA₅-Ac} (25 nM) (g) DENPs for 2 h, respectively.

FIG. 25 shows TEM images of cellular uptake of Au DENPs. a-c, TEM images of KB cells with high-level FAR treated with {(Au⁰)_{51.2}-G5-FI₅-FA₅-Ac} (a and b) and {(Au⁰)_{51.2}-G5-FI₅-Ac} DENPs (c) for 2 h, respectively. b, A magnified area of the lysosome of the same cell shown in (a). The concentration for both Au DENPs is maintained at 50 μM.

FIG. 26 shows TEM image of the uptake of {(Au⁰)_{51.2}-G5-FI₅-FA₅-Ac} DENPs in the vacuoles of KB cells.

FIG. 27 shows TEM image of the uptake of {(Au⁰)_{51.2}-G5-FI₅-FA₅-Ac} DENPs in the nuclei of KB cells.

FIG. 28 shows TEM image of the minimal non-specific uptake of {(Au⁰)_{51.2}-G5-FI₅-Ac} DENPs in the vacuole of some KB cells.

FIG. 29 shows UV-Vis spectra of the formed {(Au³⁺)₇-G5.NH₂} complexes (Curve 1), {(Au⁰)₇-G5.NHAc} DSNPs (Curve 2), {(Au³⁺)₇-G5.NH₂} complexes after reaction with glycidol for 24 h (Curve 3), {(Au⁰)₇-G5.NGlyOH} DSNPs (Curve 4), and {(Au⁰)₇-G5.NGlyOH} DSNPs formed by addition of additional glycidol. Inset shows the photographs of the corresponding solutions of {(Au³⁺)₇-G5.NH₂} complexes (1), {(Au⁰)₇-G5.NHAc} DSNPs (2), and {(Au⁰)₇-G5.NGlyOH} DSNPs (3).

FIG. 30 shows a photograph of Au NP suspensions prepared using preformed G5.NHAc dendrimers as stabilizers: (1) simply mixing G5.NHAc dendrimers with HAuCl₄; (2) the mixture of G5.NHAc dendrimers and HAuCl₄ was added with triethylamine; (3) the mixture of G5.NHAc dendrimers, HAuCl₄, and triethylamine was added with acetic anhydride.

FIG. 31 shows a TEM image (a), size distribution histogram (b), and photograph (c) of the suspension of {(Au⁰)₇-G5.NGlyOH} DSNPs prepared in the presence of free glycidol molecules.

FIG. 32 shows ¹H NMR spectra of G5.NH₂ dendrimers (a); {(Au³⁺)₇-G5.NH₂} complexes (b); G5.NHAc dendrimers (c); and {(Au⁰)₇-G5.NHAc} DSNPs (d). A schematic representation of the dendrimer structure used for NMR assignment is shown at the bottom.

FIG. 33 shows ¹H NMR spectra of G5.NGlyOH dendrimers (a) and {(Au⁰)₇-G5.NGlyOH} DSNPs. A schematic representation of the dendrimer structure used for NMR assignment is shown at the bottom.

FIG. 34 shows TEM image (a), size distribution histogram (b), selected area electron diffraction pattern (c), and an EDS spectrum (d) of {(Au⁰)₇-G5.NHAc} DSNPs.

FIG. 35 shows TEM image (a), size distribution histogram (b), and high-resolution TEM image (c) of {(Au⁰)₇-G5.NGlyOH} DSNPs.

FIG. 36 shows a) UV-Vis spectra of G5.NHAc-FI (Curve 1), G5.NHAc-FI-FA (Curve 2) dendrimers, and {(Au⁰)₇-G5.NHAc-FI} (Curve 3), {(Au⁰)₇-G5.NHAc-FI-FA} (Curve 4) DSNPs. (b) A photograph (from left to right) shows the water solution of G5.NHAc-FI, G5.NHAc-FI-FA dendrimers, and {(Au⁰)₇-G5.NHAc-FI}, {(Au⁰)₇-G5.NHAc-FI-FA} DSNPs.

FIG. 37 shows ¹H NMR spectra of {(Au⁰)₇-G5.NHAc-FI} (a) and {(Au⁰)₇-G5.NHAc-FI-FA} (b) DSNPs.

FIG. 38 shows large scale (a) and magnified (b) TEM images of {(Au⁰)₇-G5.NHAc-FI} DSNPs. (c) shows the size distribution histogram of the same DSNPs.

FIG. 39 shows large scale (a) and magnified (b) TEM images of {(Au⁰)₇-G5.NHAc-FI-FA}DSNPs. (c) shows the size distribution histogram of the same DSNPs.

FIG. 40 shows flow cytometric characterization of the dose-dependent binding of {(Au⁰)₇-G5.NHAc-FI} and {(Au⁰)₇-G5.NHAc-FI-FA} DSNPs with KB cells expressing high-(a) and low-(b) levels of folate receptor (FAR), respectively.

FIG. 41 shows confocal microscopic images of KB cells with high-level FAR treated with PBS buffer (a), {(Au⁰)₇-G5.NHAc-FI} (25 nM) (b), and {(Au⁰)₇-G5.NHAc-FI-FA} (25 mM) (c) DSNPs for 2 h, respectively.

FIG. 42 shows an MTT assay of KB cell viability after treatment with {(Au⁰)₇-G5.NHAc-FI} and {(Au⁰)₇-G5.NHAc-FI-FA} DSNPs for 24 h.

### DEFINITIONS

To facilitate an understanding of the present invention, a number of terms and phrases are defined below:

As used herein, the term “agent” refers to a composition that possesses a biologically relevant activity or property. Biologically relevant activities are activities associated with biological reactions or events or that allow the detection, monitoring, or characterization of biological reactions or events. Biologically relevant activities include, but are not limited to, therapeutic activities (e.g., the ability to improve biological health or prevent the continued degeneration associated with an undesired biological condition), targeting activities (e.g., the ability to bind or associate with a biological molecule or complex), monitoring activities (e.g., the ability to monitor the progress of a biological event or to monitor changes in a biological composition), imaging activities (e.g., the ability to observe or otherwise detect biological compositions or reactions), and signature identifying activities (e.g., the ability to recognize certain cellular compositions or conditions and produce a detectable response indicative of the presence of the composition or condition). The agents of the present invention are not limited to these particular illustrative examples. Indeed any useful agent may be used including agents that deliver or destroy biological materials, cosmetic agents, and the like. In preferred embodiments of the present invention, the agent or agents are associated with at least one dendrimer (e.g., incorporated into the dendrimer, surface exposed on the dendrimer, etc.). In some embodiments of the present invention, one dendrimer is associated with two or more agents that are different than” each other (e.g., one dendrimer associated with a targeting agent and a therapeutic agent). “Different than” refers to agents that are distinct from one another in chemical makeup and/or functionality.

As used herein, the terms “dendrimer encapsulated nanoparticle” and “DENP” refer generally to a nanostructure where one dendrimer molecule entraps one or more nanoparticles (e.g., metal nanoparticles). As used herein, the terms “dendrimer stabililzed nanoparticle” and “DSNP” refer generally to a nanostructure where one nanoparticle is stabilized by multiple dendrimer molecules.

As used herein, the term “functionalized dendrimer nanoparticle” refers to a “functionalized dendrimer encapsulated nanoparticle” and/or a “functionalized dendrimer stabilized nanoparticle.”

As used herein, the terms “functionalized dendrimer encapsulated nanoparticle” and “functionalized DENP” refer generally to a dendrimer encapsulated nanoparticle wherein charge reducing molecules (e.g., acetamide and hydroxyl) have been substituted for terminal amine groups present within the dendrimer component of the dendrimer encapsulated nanoparticle. The present invention is not limited to acetamide and hydroxyl groups. Indeed, any molecule (e.g., charge reducing molecule) that can be substituted for terminal amine groups and that reduces the overall net charge of the dendrimer encapsulated nanoparticle finds use in the present invention.

As used herein, the terms “functionalized dendrimer stabilized nanoparticle” and “functionalized DSNP” refer generally to a dendrimer stabilized nanoparticle wherein charge reducing molecules (e.g., acetamide and hydroxyl) have been substituted for terminal amine groups present within the dendrimer component of the dendrimer stabilized nanoparticle. The present invention is not limited to acetamide and hydroxyl groups. Indeed, any molecule (e.g., charge reducing molecule) that can be substituted for terminal amine groups and that reduces the overall net charge of the dendrimer stabilized nanoparticle finds use in the present invention.

As used herein, the term “nanodevice” refers to small (e.g., invisible to the unaided human eye) compositions containing or associated with one or more “agents.” In its simplest form, the nanodevice consists of a physical composition (e.g., a dendrimer or a dendrimer encapsulated nanoparticle) associated with at least one agent that provides biological functionality (e.g., a therapeutic agent). However, the nanodevice may comprise additional components (e.g., additional dendrimers and/or agents). In preferred embodiments of the present invention, the physical composition of the nanodevice comprises at least one dendrimer encapsulated nanoparticle (e.g., an acetamide-functionalized and/or hyrdroxyl functionalized denandrimer encapsulated nanoparticle) with biological functionality provided by at least one agent associated with a dendrimer.

The term “biologically active,” as used herein, refers to a protein or other biologically active molecules (e.g., catalytic RNA or small molecule) having structural, regulatory, or biochemical functions of a naturally occurring molecule.

The term “agonist,” as used herein, refers to a molecule which, when interacting with a biologically active molecule, causes a change (e.g., enhancement) in the biologically active molecule, which modulates the activity of the biologically active molecule. Agonists may include proteins, nucleic acids, carbohydrates, or any other molecules which bind or interact with biologically active molecules. For example, agonists can alter the activity of gene transcription by interacting with RNA polymerase directly or through a transcription factor.

The terms “antagonist” or “inhibitor,” as used herein, refer to a molecule which, when interacting with a biologically active molecule, blocks or modulates the biological activity of the biologically active molecule. Antagonists and inhibitors may include proteins, nucleic acids, carbohydrates, or any other molecules that bind or interact with biologically active molecules. Inhibitors and antagonists can effect the biology of entire cells, organs, or organisms (e.g., an inhibitor that slows tumor growth).

The term “modulate,” as used herein, refers to a change in the biological activity of a biologically active molecule. Modulation can be an increase or a decrease in activity, a change in binding characteristics, or any other change in the biological, functional, or immunological properties of biologically active molecules.

The term “gene” refers to a nucleic acid (e.g., DNA) sequence that comprises coding sequences necessary for the production of a polypeptide or precursor. The polypeptide can be encoded by a full length coding sequence or by any portion of the coding sequence so long as the desired activity or functional properties (e.g., enzymatic activity, ligand binding, signal transduction, etc.) of the full-length or fragment are retained. The term also encompasses the coding region of a structural gene and the including sequences located adjacent to the coding region on both the 5′ and 3′ ends for a distance of about 1 kb or more on either end such that the gene corresponds to the length of the full-length mRNA. The sequences that are located 5′ of the coding region and which are present on the mRNA are referred to as 5′ non-translated sequences. The sequences that are located 3′ or downstream of the coding region and which are present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene which are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the terms “nucleic acid molecule encoding,” “DNA sequence encoding,” and “DNA encoding” refer to the order or sequence of deoxyribonucleotides along a strand of deoxyribonucleic acid. The order of these deoxyribonucleotides determines the order of amino acids along the polypeptide (protein) chain. The DNA sequence thus codes for the amino acid sequence.

The term “antigenic determinant” as used herein refers to that portion of an antigen that makes contact with a particular antibody (e.g., an epitope). When a protein or fragment of a protein is used to immunize a host animal, numerous regions of the protein may induce the production of antibodies which bind specifically to a given region or three-dimensional structure on the protein; these regions or structures are referred to as antigenic determinants. An antigenic determinant may compete with the intact antigen (e.g., the “immunogen” used to elicit the immune response) for binding to an antibody.

The terms “specific binding” or “specifically binding” when used in reference to the interaction of an antibody and a protein or peptide means that the interaction is dependent upon the presence of a particular structure (e.g., the antigenic determinant or epitope) on the protein; in other words the antibody is recognizing and binding to a specific protein structure rather than to proteins in general. For example, if an antibody is specific for epitope “A,” the presence of a protein containing epitope A (or free, unlabelled A) in a reaction containing labelled “A” and the antibody will reduce the amount of labelled A bound to the antibody.

The term “transgene” as used herein refers to a foreign gene that is placed into an organism by, for example, introducing the foreign gene into newly fertilized eggs or early embryos. The term “foreign gene” refers to any nucleic acid (e.g., gene sequence) that is introduced into the genome of an animal by experimental manipulations and may include gene sequences found in that animal so long as the introduced gene does not reside in the same location as does the naturally-occurring gene.

As used herein, the term “vector” is used in reference to nucleic acid molecules that transfer DNA segment(s) from one cell to another. The term “vehicle” is sometimes used interchangeably with “vector.” Vectors are often derived from plasmids, bacteriophages, or plant or animal viruses.

The term “expression vector” as used herein refers to a recombinant DNA molecule containing a desired coding sequence and appropriate nucleic acid sequences necessary for the expression of the operably linked coding sequence in a particular host organism. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals.

As used herein, the term “gene transfer system” refers to any means of delivering a composition comprising a nucleic acid sequence to a cell or tissue. For example, gene transfer systems include, but are not limited to vectors (e.g., retroviral, adenoviral, adeno-associated viral, and other nucleic acid-based delivery systems), microinjection of naked nucleic acid, and polymer-based delivery systems (e.g., liposome-based and metallic particle-based systems). As used herein, the term “viral gene transfer system” refers to gene transfer systems comprising viral elements (e.g., intact viruses and modified viruses) to facilitate delivery of the sample to a desired cell or tissue. As used herein, the term “adenovirus gene transfer system” refers to gene transfer systems comprising intact or altered viruses belonging to the family Adenoviridae.

The term “transfection” as used herein refers to the introduction of foreign DNA into eukaryotic cells. Transfection may be accomplished by a variety of means known to the art including calcium phosphate-DNA co-precipitation, DEAE-dextran-mediated transfection, polybrene-mediated transfection, electroporation, microinjection, liposome fusion, lipofection, protoplast fusion, retroviral infection, and biolistics.

As used herein, the term “cell culture” refers to any in vitro culture of cells. Included within this term are continuous cell lines (e.g., with an immortal phenotype), primary cell cultures, finite cell lines (e.g., non-transformed cells), and any other cell population maintained in vitro.

As used herein, the term “in vitro” refers to an artificial environment and to processes or reactions that occur within an artificial environment. In vitro environments can consist of, but are not limited to, test tubes and cell culture. The term “in vivo” refers to the natural environment (e.g., an animal or a cell) and to processes or reaction that occur within a natural environment.

The term “test compound” refers to any chemical entity, pharmaceutical, drug, and the like that can be used to treat or prevent a disease, illness, sickness, or disorder of bodily function. Test compounds comprise both known and potential therapeutic compounds. A test compound can be determined to be therapeutic by screening using the screening methods of the present invention. A “known therapeutic compound” refers to a therapeutic compound that has been shown (e.g., through animal trials or prior experience with administration to humans) to be effective in such treatment or prevention.

The term “sample” as used herein is used in its broadest sense and includes environmental and biological samples. Environmental samples include material from the environment such as soil and water. Biological samples may be animal, including, human, fluid (e.g., blood, plasma and serum), solid (e.g., stool), tissue, liquid foods (e.g., milk), and solid foods (e.g., vegetables).

As used herein, the terms “photosensitizer,” and “photodynamic dye,” refer to materials which undergo transformation to an excited state upon exposure to a light quantum. Examples of photosensitizers and photodynamic dyes include, but are not limited to, Photofrin 2, benzoporphyrin, m-tetrahydroxyphenylchlorin, tin etiopurpurin, copper benzochlorin, and other porphyrins.

The present invention relates to compositions comprising functionalized dendrimer-stabilized nanoparticles, functionalized dendrimer-encapsulated nanoparticles, and methods of generating and using the same.

Metal (e.g., gold or “Au”) nanoparticles (NPs) have recently received immense scientific and technological interest because of their extensive applications in biology, catalysis, and nanotechnology. Recent advances in biologic nanotechnology show that metal (e.g., gold, platinum, etc.) nanoparticles (NPs) can be used as platforms for biodiagnosis, biosensing, gene delivery carriers, and targeted drug delivery (See, e.g., Tkachenko et al., J. Am. Chem. Soc. 2003, 125, 4700-4701; Rosi and Mirkin, Chem. Rev. 2005, 105, 1547-1562; Daniel and Astruc, Chem. Rev. 2004, 104, 293-346; Parak et al., Nanotechnology 2003, 14, R15-R27). In most cases, the surface modification and engineering of metal NPs is performed before they are applied in biological systems. For example, peptides (See, e.g., Tkachenko et al., J. Am. Chem. Soc. 2003, 125; Aubin et al., Nano Lett. 2005, 5, 519-522; Tkachenko et al., Bioconjugate Chem. 2004, 15, 482-490) and proteins (See, e.g., E1-Sayed et al., Nano Lett. 2005, 5, 829-834; Yang et al., Bioconjugate Chem. 2005, 16, 494-496) have been conjugated onto gold (Au⁰) NPs for targeting purposes. Antibody has been conjugated onto Au NP surfaces for immunoassay and biosensing (See, e.g., Thanh and Rosenzweig, Anal. Chem. 2002, 74, 1624-1628; Ho et al., Anal. Chem. 2004, 76, 7162-7168). DNA-modified Au NPs can be used as nanoprobes to detect target DNA sequences through hybridization (See, e.g., Rosi and Mirkin, Chem. Rev. 2005, 105, 1547-1562; Parak et al., Nanotechnology 2003, 14, R15-R27). Importantly, the Au NPs used for bioconjugation have, to date, been prepared by citric acid reduction and protection under an elevated temperature. Thio chemistry has been extensively utilized in the conjugation of Au NPs with various biological ligands (See, e.g., Rosi and Mirkin, Chem. Rev. 2005, 105, 1547-1562; Daniel and Astruc, Chem. Rev. 2004, 104, 293-346).

In order to achieve various applications, the metal NP surfaces have been modified with different functionalities. For instance, alkanethiol (See, e.g., Lala et al., Langnmuir 2001, 17, 3766-3768) and alkylamine (See, e.g., Mayya and Caruso, Langmuir 2003, 19, 6987-6993; Cheng and Wang, J. Phys. Chem. B. 2004, 108, 24-26; Liu et al., J. Am. Chem. Soc. 2001, 123, 11148-11154) molecules have been used for phase transfer of gold NPs and oligonucleotides have been linked onto gold NPs for subsequent spatial organization of nanocrystals and detection of particular DNA sequences (See, e.g., Mirkin, MRS Bull. 2000, 25, 43-54; Niemeyer, Curr. Opin. Chem. Biol. 2000, 4, 609-6183; Zanchet et al., J. Phys. Chem. B 2002, 106, 11758-11763; Parak et al., Nano Lett. 2003, 3, 33-36).

An alternative approach to prepare metal NPs has been developed by using dendrimers (e.g., polyamidoamine (PAMAM) dendrimers) as templates or stabilizers (See, e.g., Balogh and Tomalia, J. Am. Chem. Soc. 1998, 120, 7355-7356; Esumi et al., Langmuir 1998, 14, 3157-3159; Crooks et al., Accounts Chem. Res. 2001, 34, 181-190; Zhao et al., J. Am. Chem. Soc. 1998, 120, 4877-4878). Dendrimers (e.g., PAMAM dendrimers) are close to spherical, highly branched macromolecules with symmetrically emanating dendrons of defined molecular weight and size (See, e.g., U.S. Pat. No. 6,471,968, and U.S. Pat. App. Nos. 60/604,321, filed Aug. 25, 2004, and 60/690,652, filed Jun. 15, 2005, herein incorporated by reference in their entireties). They are composed of a core molecule and dendritic branches that regularly extend from the core to terminal groups (See, Tomalia et al., Polymer J. 1985, 17, 117; Tomalia et al., Macromolecules 1986, 19, 2466-2468; Tomalia et al., Angew. Chem. Int. Ed. Engl. 1990, 29, 138). Dendrimers (e.g., PAMAMs) have a narrow polydispersity and are ideal stabilizers to encapsulate and stabilize metal nanoparticles due to their “built-in” functional groups, fairly uniform composition and defined structures. Organic/inorganic hybrid metal dendrimer NPs hold great promise in various applications such as catalysis (See, e.g., Zhao and Crooks, Angew. Chem. Int. Ed. 1999, 38, 364-366), optics (See, e.g., Ispasoiu et al., J. Am. Chem. Soc. 2000, 122, 11005-11006; Ye et al., Appl. Phys. Lett. 2002, 80, 1713-1715), biological sensing (See, e.g., Bielinska et al., J. Nanoparticle Res. 2002, 4, 395-403), cancer therapeutics (See, e.g., Balogh et al., Chimica Oggi/Chemistry Today 2002, 20, 35-40), and building blocks to assemble functional films (See, e.g., He et al., Chem. Mater. 1999, 11, 3268-3274; Esumi et al., Langmuir 2003, 19, 7679-7681).

The preparation of dendrimer-stabilized metal (e.g., gold) nanoparticles (Au DSNPs) usually involves complexation of gold salts (e.g. HAuCl₄) with PAMAM dendrimers, followed by physical or chemical reduction (See, e.g., Crooks et al., Accounts Chem. Res. 2001, 34, 181-190; Esumi, K. Topics in Current Chemistry (Colloid Chemistry II) 2003, 227, 31-52; Esumi, K. Encyclopedia of Nanoscience and Nanotechnology 2004, 2, 317-326; Balogh et al., J. Nanoparticle Res. 1999, 1, 353-368). Dendrimer encapsulated/stabilized metal NPs have been described (See, e.g., Balogh and Tomalia, J. Am. Chem. Soc. 1998, 120, 7355-7356; Esumi et al., Langmuir 1998, 14, 3157-3159; Zhao et al., J. Am. Chem. Soc. 1998, 120, 48774878) as well as specifically regarding Au DSNPs (See, e.g., Esumi et al., Langmuir 1998, 14, 3157-3159; Balogh et al., J. Nanoparticle Res. 1999, 1, 353-368; Garcia et al., Anal. Chem. 1999, 71, 256-258; Grohn et al., Macromolecules 2000, 33, 6042-6050). The size of the Au DSNPs is mainly dependent on the molar ratio between dendrimers and Au atoms (See, e.g., Esumi, K. Topics in Current Chemistry (Colloid Chemistry II) 2003, 227, 31-52). Mechanistic studies have shown that dendrimer terminal amines are extremely effective in the stabilization of Au NPs (See, Garcia et al., Anal. Chem. 1999, 71, 256-258; Manna et al., Chem. Mater. 2001, 13, 1674-1681).

Although studies have been performed regarding their synthesis and potential applications, the study of generation-dependent structure and properties of Au DSNPs using systematic characterization techniques have yet to be fully investigated. For example, an understanding of the formed nanohybrid structures from different aspects of view still remains a great challenge (See, e.g., Grohn et al., Macromolecules 2000, 33, 6042-6050; Manna et al., Chem. Mater. 2001, 13, 1674-1681).

One approach to the preparation of metal (e.g., Au) NPs is through the use of PAMAM dendrimers as templates (See, e.g., Crooks et al., Acc. Chem. Res.; 2001, 34, 181-190; Balogh et al., J. Nanoparticle Res. 1999, 1; 353-368; Esumi et al., Langmuir 1998, 14, 3157-3159; Esumi, K. Topics in Current Chemistry 2003, 227, 31-52). Dendrimer-encapsulated NPs (e.g., Au DENPs) have been prepared using amine-terminated PAMAM dendrimers because of their commercial availability. However, due to the high in vivo toxicity of amine-terminated PAMAM dendrimers (See, e.g., Balogh et al., Pharma Chem 2003, 2, 94-99), the subsequent {(Au⁰)ₙ—PAMAM} DENPs have been extremely limited in their biological applications. For example, to date, metal DENPs have not enjoyed broad, biological applications because the DENPs possess an overall positive charge (e.g. due to the amine groups present at the terminal ends of the dendrimers) that is not compatible with in vivo applications (e.g., they are toxic).

Accordingly, the present invention provides compositions comprising and methods of generating functionalixed {(Au⁰), —PAMAM} DSNPs and/or functionalized DENPs that overcome incompatibility issues with biological systems. For example, in preferred embodiments, the functionalized DSNPs and/or functionalized DENPs are non-toxic (e.g., when administered to a subject) due to reduction of overall charge of the DSNPs and/or DENPs (e.g., using methods of the present invention).

Although an understanding of the mechanism is not necessary to practice the present invention and the present invention is not limited to any particular mechanism of action, in some embodiments, decreasing the surface charge of amine-terminated PAMAM dendrimers (e.g., towards or to neutral) reduces their in vivo toxicity. For example, in some embodiments, decreasing (e.g., neutralizing) the surface charge of amino-terminated PAMAM dendrimers is achieved by acetylation and/or hydroxylation of the PAMAM terminal amine groups (See, e.g., Examples 2-3 and 4-5), although the present invention is not limited to acetylation or hydroxylation.

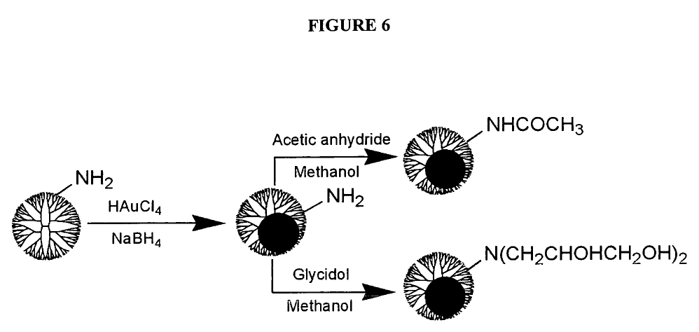

Thus, in some embodiments, the present invention provides a method of generating functionalized dendrimer-stabilized nanoparticles (DSNPs) or functionalize dendrimer-encapsulated nanoparticles (e.g., Au DENPs) with different functional groups (e.g., through the modification of the terminal amine groups of PAMAM templates, See Examples 2 and 4). For example, in some embodiments, DENPs (e.g., Au DENPs preformed using amine-terminated PAMAM dendrimers) are used as templates and reacted with acetic anhydride molecules to form acetamide-functionalized Au DENPs (See Example 2 and FIG. 6). In some embodiments, DENPs (e.g., Au DENPs) are used as templates and reacted with glycidol molecules to form hydroxyl-functionalized Au DENPs (See Example 2 and FIG. 6). In some embodiments, the DENPs are first conjugated to one or more functional groups (e.g., a therapeutic agent, an imaging agent, a targeting agent, or a combination thereof) prior to reaction with glycidol and/or acetic anhydride. The present invention is not limited by the nature of the agent used to reduce the charge of the DENPs (e.g., that adds one or more molecules—e.g., hydroxyl or acetamide molecules—to terminal amines of the DENPs). Indeed, any agent that can be used to reduce the net charge of the DENPs is contemplated to be useful in the present invention. In some embodiments, generation 5 dendrimers (G5.NH₂, also referred to herein as E5.NH₂ with E denoting the ethylenediamine core of the dendrimer) are used as preformed, amine-terminated PAMAM dendrimers. In some embodiments, the dendrimer is a generation 5 (G5) polyamideamine (PAMAM) or polypropylamine (POPAM) dendrimer, although the present invention is not limited to any particular generation or chemistry used to generate the dendrimers. For example, in some embodiments, the dendrimer is a G3 dendrimer, a G4, dendrimer, a G5 dendrimer, a G6 dendrimer, a G7 dendrimer, a G8 denrimer, or a dendrimer of a generation greater than 8 or less than 3. In some embodiments, the composition comprises a dendron (See, e.g., Al-Jamal et al., Adv Drug Deliv Rev. 2005 57(15):2238-2270) rather than or in addition to the dendrimer.

In some embodiments, the present invention provides a one-step synthesis of DSNPs with acetamide or hydroxyl surfaces via acetylation of dendrimer-metal complexes (e.g., dendrimer-Au(III) complexes) or by mixing glycidol hydroxylated dendrimers with metal (e.g., Au) anions, respectively (See Examples 5 and 6). In some embodiments, changing the molar ratio between dendrimers and metal atoms can be used to control the size of the DSNPs. In some embodiments, utilizing dendrimers of different generations may be used to control the size of the DSNPs. In some embodiments, the average size of a DSNP is about 13 nm. However, the present invention is not limited to any particular size of DSNP. Indeed, a variety of sizes of DSNPs find use in the present invention including, but not limited to, DSNPs smaller than 13 nm (e.g., averaging 11 nm, 10 nm, 9 nm, 8 nm, or less) or larger than 13 nm (e.g., 14 nm, 15 mm, 17 nm, 20 nm or more).

The present invention provides a novel method of modifying DENPs and/or DSNPs (e.g., Au DENPs and/or DSNPs) surfaces through dendrimer-mediated conventional organic reactions. The formed DENPs and/or DSNPs (e.g., Au DENPs and/or DSNPs) after surface functionalization are stable, water-soluble, and display similar size distributions and optical properties, while the surface charge polarity can be changed (e.g., using the compositions and methods of the present invention, See, e.g., Examples 1-3). Accordingly, in some embodiments, the present invention provides the ability to directly tailor the surface functionalities of preformed Au DENPs and/or DSNPs. Compositions comprising functionalized DENPs and/or DSNPs (e.g., Au DENPs and/or DSNPs) of the present invention find use in a variety of settings including, but not limited to, therapeutic, diagnostic and research applications.

For example, in some embodiments, the functionalized DENPs and/or DSNPs of the present invention find use as a nanoplatform for targeting, imgaing, and/or treatment of cancers; drug delivery; biosensing (e.g., imaging a target); catalysis; optics; as well as development of novel functional materials.

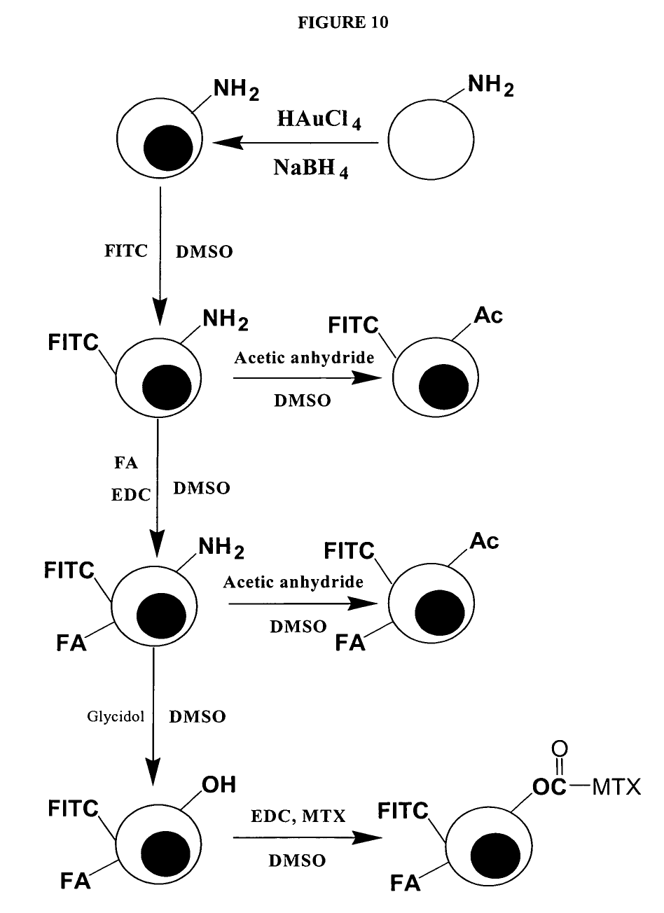

In some embodiments, hydroxyl groups (e.g., present within a functionalized DENP and/or DSNP of the present invention) are used for linking functional groups (e.g., a therapeutic agent, an imaging agent, or a targeting agent) to the DENP and/or DSNP. In some embodiments, DENPs and/or DSNPs (e.g., Au DENPs and/or DSNPs) with terminal amine groups can be modified (e.g., conjugated to) with various functional groups (e.g., a therapeutic agent, a drug or pharmaceutical, an imaging agent, or a targeting agent), with the remaining amine groups (e.g., amine groups not conjugated to a functional group) transferred to acetamide or hydroxyl groups (e.g., using the compositions and methods of the present invention) to decrease the total surface charge. Although an understanding of the mechanism is not necessary to practice the present invention and the present invention is not limited to any particular mechanism of action, neutralization of the functionalized metal DENPs decrease the non-specific binding and toxicity of the DENPs and/or DSNPs. In some embodiments, more than one functional group is added to the DENPs and/or DSNPs (e.g., multiple targeting agents, multiple therapeutic agents or multiple imaging agents, or a combination thereof, See, e.g., FIG. 10). In some embodiments, DENPs and/or DSNPs of the present invention are modified with (e.g., conjugated to) one or more targeting agents, one or more therapeutic agents and/or one or more imaging agents (See, Examples 4 and 6).

In some embodiments, the present invention provides a method of generating a functionalized, non-toxic DENPs and/or DSNPs (e.g., Au DENPs and/or DSNPs) comprising one or more functional groups. In some embodiments, DENPs and/or DSNPs are conjugated to functional groups (e.g., conjugated to therapeutic agents, imaging agents and/or targeting agents), and, once the functional groups are added, acetamide and/or hydroxyl molecules are added (e.g., via methods of the present invention using acetic anhydride and/or glycidol). Although an understanding of the mechanism is not necessary to practice the present invention and the present invention is not limited to any particular mechanism of action, in some embodiments, addition of acetamide and/or hydroxyl groups to non-reacted amine groups (e.g., amine groups not conjugated to a functional group) decreases the overall surface charge of the DENPs and/or DSNPs (e.g., by modifying the amine groups to hydroxyl or acetamide groups, thereby decreasing the toxicity).

The present invention is not limited by the order in which the functional groups and hydroxyl/acetamide groups are added. In some embodiments, one or more functional groups are added to (e.g., conjugated to terminal amine groups of) DENPs and/or DSNPs (e.g., Au DENPs and/or DSNPs) and then hydroxyl/acetamide groups are added (e.g., via organic reactions involving glycidol and/or acetic anhydride) to the non-conjugated amine groups. In other embodiments, one or more functional groups are added to DENPs and/or DSNPs, followed by addition of hydroxyl groups to the non-conjugated amine groups. In other embodiments, one or more functional groups are added to DENPs and/or DSNPs, followed by addition of hydroxyl groups to the non-conjugated amine groups, followed by addition of one or more functional groups to the terminal hydroxyl groups.

Thus, in some embodiments, depending upon the nature of the functional groups (e.g., therapeutic agents, imaging agents and/or targeting agents), DENPs and/or DSNPs may be conjugated to one or more functional groups (e.g., an imaging agent and/or a targeting agent and/or a therapeutic agent), followed by hydroxylation of amine groups of the DENPs and/or DSNPs (e.g., via exposure to glycidol in the organic synthesis reaction), followed by conjugation of one or more functional groups (e.g., a therapeutic agent such as a chemotherapeutic or other drug) by conjugating to the newly added hydroxyl groups.

In some embodiments, the functionalized DENPs and/or DSNPs of the present invention are used in treatment and/or monitoring during cancer therapy. However, the systems and compositions of the present invention find use in the treatment and monitoring of a variety of disease states or other physiological conditions, and the present invention is not limited to use with any particular disease state or condition. Other disease states that find particular use with the present invention include, but are not limited to, cardiovascular disease, viral disease, inflammatory disease, and other proliferative disorders.

Some embodiments of the present invention provide compositions comprising a DENPs and/or DSNPs conjugated to one or more functional groups, the functional groups including, but not limited to, therapeutic agents, biological monitoring components, biological imaging components, targeting components, and components to identify the specific signature of cellular abnormalities. As such, the therapeutic nanodevice is made up of individual dendrimers, each with one or more functional groups being specifically conjugated with or covalently linked to the DENPs and/or DSNPs (See Examples 4-6).

The following discussion describes individual component parts of the dendrimer and methods of making and using the same in some embodiments of the present invention. To illustrate the design and use of the systems and compositions of the present invention, the discussion focuses on specific embodiments of the use of the compositions in the treatment and monitoring of breast adenocarcinoma and colon adenocarcinoma. These specific embodiments are intended only to illustrate certain preferred embodiments of the present invention and are not intended to limit the scope thereof (e.g., compositions and methods of the present invention find use in the identification and treatment of prostate cancer and virally infected cells and tissue). In some embodiments, the DENPs and/or DSNPs of the present invention target neoplastic cells through cell-surface moieties and are taken up by the tumor cell for example through receptor mediated endocytosis. In preferred embodiments, an imaging component (e.g., conjugated to a dendrimer of the present invention) allows the tumor to be imaged (e.g., through the use of MRI).

In some embodiments, the release of a therapeutic agent is facilitated by the therapeutic component being attached to a labile protecting group, such as, for example, cisplatin being attached to a photolabile protecting group that becomes released by laser light directed at those cells emitting the color of fluorescence activated as mentioned above (e.g., red-emitting cells). Optionally, the therapeutic device (e.g., compositions comprising DENPs and/or DSNPs of the present invention) also may have a component to monitor the response of a target cell or tissue (e.g., a tumor) to therapy. For example, where a chemotherapeutic agent (e.g., methotrexate) conjugated to a DENP and/or DSNP of the present invention induces apoptosis of a targeted cell, the caspase activity of the targeted cells may be used to activate a green fluorescence. This allows apoptotic cells to turn orange, (combination of red and green) while residual cells remain red. Any normal cells that are induced to undergo apoptosis in collateral damage fluoresce green.

As is clear from the above example, the use of the compositions of the present invention facilitates non-intrusive sensing, signaling, and intervention for cancer and other diseases and conditions. Since specific protocols of molecular alterations in cancer cells are identified using this technique, non-intrusive sensing through the DSNPs and/or DENPs is achieved and may then be employed automatically against various tumor phenotypes.

### Therapeutic Agents

A wide range of therapeutic agents find use with the present invention. Accordingly, the present invention is not limited by the type of therapeutic agent(s) that may be conjugated to a DENP AND/OR DSNP and/or DSNP of the present invention. Any therapeutic agent that can be associated with a dendrimer may be delivered using the methods, systems, and compositions of the present invention. To illustrate delivery of therapeutic agents, the following discussion focuses mainly on the delivery of methotrexate, cisplatin and taxol for the treatment of cancer. Also discussed are various photodynamic therapy compounds, and various antimicrobial compounds.

i. Methotrexate, Cisplatin and Taxol

The cytotoxicity of methotrexate depends on the duration for which a threshold intracellular level is maintained (Levasseur et al., Cancer Res 58, 5749 (1998); Goldman & Matherly, Pharmacol Ther 28, 77 (1985)). Cells contain high concentrations of DHFR, and, to shut off the DHFR activity completely, anti-folate levels six orders of magnitude higher than the Ki for DHFR is required (Sierrra & Goldman, Seminars in Oncology 26, 11 (1999)). Furthermore, less than 5% of the enzyme activity is sufficient for full cellular enzymatic function (White & Goldman, Biol Chem 256, 5722 (1981)). Cisplatin and Taxol have a well-defined action of inducing apoptosis in tumor cells (See e.g., Lanni et al., Proc. Natl. Acad. Sci., 94:9679 (1997); Tortora et al., Cancer Research 57:5107 (1997); and Zaffaroni et al., Brit. J. Cancer 77:1378 (1998)). However, treatment with these and other chemotherapeutic agents is difficult to accomplish without incurring significant toxicity. The agents currently in use are generally poorly water soluble, quite toxic, and given at doses that affect normal cells as wells as diseased cells. For example, paclitaxel (Taxol), one of the most promising anticancer compounds discovered, is poorly soluble in water.

Paclitaxel has shown excellent antitumor activity in a wide variety of tumor models such as the B16 melanoma, L1210 leukemias, MX-1 mammary tumors, and CS-1 colon tumor xenografts. However, the poor aqueous solubility of paclitaxel presents a problem for human administration. Accordingly, currently used paclitaxel formulations require a cremaphor to solubilize the drug. The human clinical dose range is 200-500 mg. This dose is dissolved in a 1:1 solution of ethanol:cremaphor and diluted to one liter of fluid given intravenously. The cremaphor currently used is polyethoxylated castor oil. It is given by infusion by dissolving in the cremaphor mixture and diluting with large volumes of an aqueous vehicle. Direct administration (e.g., subcutaneous) results in local toxicity and low levels of activity. Thus, there is a need for more efficient and effective delivery systems for these chemotherapeutic agents.

The present invention overcomes these problems by providing methods and compositions for specific drug delivery. The present invention also provides the ability to administer combinations of agents (e.g., two or more different therapeutic agents) to produce an additive effect. The use of multiple agents may be used to counter disease resistance to any single agent. For example, resistance of some cancers to single drugs (taxol) has been reported (Yu et al., Molecular Cell. 2:581 (1998)). Furthermore, the present invention provides the ability to administer one or more functionalized DENPs and/or DSNPs (e.g., conjugated to one or more functional groups described herein) in combination with other forms of therapeutic treatments (e.g., in combination with a chemotherapeutic treatment for cancer).

The present invention also provides compositions comprising DENPs and/or DSNPs that specifically target and bind to a target cell (e.g., a cancer cell) without binding to a non-target cell (e.g., a non-cancer cell). The ability to differentiate target cells from non-target cells permits compositions and methods comprising DENPs and/or DSNPs to be used to differentiate target cells from surrounding non-target cells and tissue.

The present invention also provides the opportunity to monitor therapeutic success following delivery of a therapeutic (e.g., methotrexate and/or cisplatin and/or Taxol) to a subject. For example, measuring the ability of these drugs to induce apoptosis in vitro is reported to be a marker for in vivo efficacy (Gibb, Gynecologic Oncology 65:13 (1997)). Therefore, in addition to the targeted delivery of either one, two or all of these drugs (or other therapeutic agents) to provide effective anti-tumor therapy and reduce toxicity, the effectiveness of the therapy can be gauged by techniques of the present invention that monitor the induction of apoptosis. Importantly, these therapeutics are active against a wide-range of tumor types including, but not limited to, breast cancer and colon cancer (Akutsu et al., Eur. J. Cancer 31A:2341 (1995)).

Although the above discussion describes three specific agents, any agent (e.g., small molecule, drug or pharmaceutical) that is routinely used in a cancer therapy context finds use in the present invention. In treating cancer according to the invention, the therapeutic component of the DENP and/or DSNP may comprise compounds including, but not limited to, adriamycin, 5-fluorouracil, etoposide, camptothecin, actinomycin-D, mitomycin C, or more preferably, cisplatin. The agent may be prepared and used as a combined therapeutic composition, or kit, by combining it with an immunotherapeutic agent, as described herein.

In some embodiments of the present invention, the DENP and/or DSNP is contemplated to comprise one or more agents that directly cross-link nucleic acids (e.g., DNA) to facilitate DNA damage leading to a synergistic, antineoplastic agents of the present invention. Agents such as cisplatin, and other DNA alkylating agents may be used. Cisplatin has been widely used to treat cancer, with efficacious doses used in clinical applications of 20 mg/M² for 5 days every three weeks for a total of three courses. The DENPs may be delivered via any suitable method, including, but not limited to, injection intravenously, subcutaneously, intratumorally, intraperitoneally, or topically (e.g., to mucosal surfaces).

Agents that damage DNA also include compounds that interfere with DNA replication, mitosis and chromosomal segregation. Such chemotherapeutic compounds include adriamycin, also known as doxorubicin, etoposide, verapamil, podophyllotoxin, and the like. Widely used in a clinical setting for the treatment of neoplasms, these compounds are administered through bolus injections intravenously at doses ranging from 25-75 Mg/M² at 21 day intervals for adriamycin, to 35-50 Mg/M² for etoposide intravenously or double the intravenous dose orally.

Agents that disrupt the synthesis and fidelity of nucleic acid precursors and subunits also lead to DNA damage and find use as chemotherapeutic agents in the present invention. A number of nucleic acid precursors have been developed. Particularly useful are agents that have undergone extensive testing and are readily available. As such, agents such as 5-fluorouracil (5-FU) are preferentially used by neoplastic tissue, making this agent particularly useful for targeting to neoplastic cells. The doses delivered may range from 3 to 15 mg/kg/day, although other doses may vary considerably according to various factors including stage of disease, amenability of the cells to the therapy, amount of resistance to the agents and the like.

The anti-cancer therapeutic agents that find use in the present invention are those that are amenable to incorporation into DENP and/or DSNP structures or are otherwise associated with DENP and/or DSNP structures such that they can be delivered into a subject, tissue, or cell without loss of fidelity of its anticancer effect. For a more detailed description of cancer therapeutic agents such as a platinum complex, verapamil, podophyllotoxin, carboplatin, procarbazine, mechlorethamine, cyclophosphamide, camptothecin, ifosfamide, melphalan, chlorambucil, bisulfan, nitrosurea, adriamycin, dactinomycin, daunorubicin, doxorubicin, bleomycin, plicomycin, mitomycin, etoposide (VP16), tamoxifen, taxol, transplatinum, 5-fluorouracil, vincristin, vinblastin and methotrexate and other similar anti-cancer agents, those of skill in the art are referred to any number of instructive manuals including, but not limited to, the Physician's Desk reference and to Goodman and Gilman's “Pharmaceutical Basis of Therapeutics” ninth edition, Eds. Hardman et al., 1996.

In some embodiments, the drugs are preferably attached to the DENP and/or DSNP with photocleavable linkers. For example, several heterobifunctional, photocleavable linkers that find use with the present invention are described by Ottl et al. (Ottl et al., Bioconjugate Chem., 9:143 (1998)). These linkers can be either water or organic soluble. They contain an activated ester that can react with amines or alcohols and an epoxide that can react with a thiol group. In between the two groups is a 3,4-dimethoxy-6-nitrophenyl photoisomerization group, which, when exposed to near-ultraviolet light (365 nm), releases the amine or alcohol in intact form. Thus, the therapeutic agent, when linked to the DENP and/or DSNP compositions of the present invention using such linkers, may be released in biologically active or activatable form through exposure of the target area to near-ultraviolet light.

In some embodiments, methotrexate is conjugated to the DENP and/or DSNP via an ester bond. In an exemplary embodiment, the alcohol group of taxol is reacted with the activated ester of the organic-soluble linker. This product in turn is reacted with the partially-thiolated surface of appropriate dendrimers (the primary amines of the dendrimers can be partially converted to thiol-containing groups by reaction with a sub-stoichiometric amount of 2-iminothiolano). In the case of cisplatin, the amino groups of the drug are reacted with the water-soluble form of the linker. If the amino groups are not reactive enough, a primary amino-containing active analog of cisplatin, such as Pt(II) sulfadiazine dichloride (See e.g., Pasani et al., Inorg. Chim. Acta 80:99 (1983) and Abel et al, Eur. J. Cancer 9:4 (1973)) can be used. Thus conjugated, the drug is inactive and will not harm normal cells. When the functionalized DENP and/or DSNP (e.g., conjugated to methotrexate) is localized within tumor cells, it is exposed to laser light of the appropriate near-UV wavelength, causing the active drug to be released into the cell.

Similarly, in other embodiments of the present invention, the amino groups of cisplatin (or an analog thereof) is linked with a very hydrophobic photocleavable protecting group, such as the 2-nitrobenzyloxycarbonyl group (See, e.g., Pillai, V. N. R. Synthesis: 1-26 (1980)). With this hydrophobic group attached, the drug is loaded into and very preferentially retained by the hydrophobic cavities within the PAMAM dendrimer (See e.g., Esfand et al., Pharm. Sci., 2:157 (1996)), insulated from the aqueous environment. When exposed to near-UV light (about 365 nm), the hydrophobic group is cleaved, leaving the intact drug. Since the drug itself is hydrophilic, it diffuses out of the DENP and/or DSNP and into the tumor cell, where it initiates apoptosis.

An alternative to photocleavable linkers are enzyme cleavable linkers. A number of photocleavable linkers have been demonstrated as effective anti-tumor conjugates and can be prepared by attaching cancer therapeutics, such as doxorubicin, to water-soluble polymers with appropriate short peptide linkers (See e.g., Vasey et al., Clin. Cancer Res., 5:83 (1999)). The linkers are stable outside of the cell, but are cleaved by thiolproteases once within the cell. In a preferred embodiment, the conjugate PK1 is used. As an alternative to the photocleavable linker strategy, enzyme-degradable linkers, such as Gly-Phe-Leu-Gly may be used.

The present invention is not limited by the nature of the therapeutic technique. For example, other conjugates that find use with the present invention include, but are not limited to, using conjugated boron clusters for BNCT (Capala et al., Bioconjugate Chem., 7:7 (1996)), the use of radioisotopes, and conjugation of toxins such as ricin to the nanodevice.

ii. Photodynamic Therapy

Photodynamic therapeutic agents may also be used as therapeutic agents in the present invention. In some embodiments, the dendrimeric compositions of the present invention containing photodynamic compounds are illuminated, resulting in the production of singlet oxygen and free radicals that diffuse out of the fiberless radiative effector to act on the biological target (e.g., tumor cells or bacterial cells). Some preferred photodynamic compounds include, but are not limited to, those that can participate in a type II photochemical reaction:

PS+hv [symbol] PS* (1)

PS*(1) [symbol] PS* (3)

PS*(3)+O₂ [symbol] PS+*O₂

*O₂+T [symbol] cytotoxicity

where PS=photosenstizer, PS*(1)=excited singlet state of PS, PS*(3)=excited triplet state of PS, hv=light quantum, *O₂=excited singlet state of oxygen, and T=biological target. Other photodynamic compounds useful in the present invention include those that cause cytotoxity by a different mechanism than singlet oxygen production (e.g., copper benzochlorin, Selman, et al., Photochem. Photobiol., 57:681-85 (1993), incorporated herein by refercnce). Examples of photodynamic compounds that find use in the present invention include, but are not limited to Photofrin 2, phtalocyanins (See e.g., Brasseur et al., Photochem. Photobiol., 47:705-11 (1988)), benzoporphyrin, tetrahydroxyphenylporphyrins, naphtalocyanines (See e.g., Firey and Rodgers, Photochem. Photobiol., 45:535-38 (1987)), sapphyrins (Sessler et al., Proc. SPIE, 1426:318-29 (1991)), porphinones (Chang et al., Proc. SPIE, 1203:281-86 (1990)), tin etiopurpurin, ether substituted porphyrins (Pandey et al., Photochem. Photobiol., 53:65-72 (1991)), and cationic dyes such as the phenoxazines (See e.g., Cincotta et al., SPIE Proc., 1203:202-10 (1990)).
iii. Antimicrobial Therapeutic Agents

Antimicrobial therapeutic agents may also be used as therapeutic agents in the present invention. Any agent that can kill, inhibit, or otherwise attenuate the function of microbial organisms may be used, as well as any agent contemplated to have such activities. Antimicrobial agents include, but are not limited to, natural and synthetic antibiotics, antibodies, inhibitory proteins, antisense nucleic acids, membrane disruptive agents and the like, used alone or in combination. Indeed, any type of antibiotic may be used including, but not limited to, anti-bacterial agents, anti-viral agents, anti-fungal agents, and the like.

### Signature Identifying Agents

In certain embodiments, the DENPs and/or DSNPs of the present invention contain one or more signature identifying agent's that are activated by, or are able to interact with, a signature component (“signature”). In preferred embodiments, the signature identifying agent is an antibody, preferably a monoclonal antibody, that specifically binds the signature (e.g., cell surface molecule specific to a cell to be targeted).

In some embodiments of the present invention, tumor cells are identified. Tumor cells have a wide variety of signatures, including the defined expression of cancer-specific antigens such as Mucd, HER-2 and mutated p53 in breast cancer. These act as specific signatures for the cancer, being present in 30% (HER-2) to 70% (mutated p53) of breast cancers. In a preferred embodiment, DENPs and/or DSNPs of the present invention comprise a monoclonal antibody that specifically binds to a mutated version of p53 that is present in breast cancer.

In some embodiments of the present invention, cancer cells expressing susceptibility genes are identified. For example, in some embodiments, there are two breast cancer susceptibility genes that are used as specific signatures for breast cancer: BRCA1 on chromosome 17 and BRCA2 on chromosome 13. When an individual carries a mutation in either BRCA1 or BRCA2, they are at an increased risk of being diagnosed with breast or ovarian cancer at some point in their lives. These genes participate in repairing radiation-induced breaks in double-stranded DNA. It is thought that mutations in BRCA1 or BRCA2 might disable this mechanism, leading to more errors in DNA replication and ultimately to cancerous growth.

In addition, the expression of a number of different cell surface receptors find use as targets for the binding and uptake of the DENPs and/or DSNPs. Such receptors include, but are not limited to, EGF receptor, folate receptor, FGR receptor 2, and the like.

In some embodiments of the present invention, changes in gene expression associated with chromosomal abborations are the signature component. For example, Burkitt lymphoma results from chromosome translocations that involve the Myc gene. A chromosome translocation means that a chromosome is broken, which allows it to associate with parts of other chromosomes. The classic chromosome translocation in Burkitt lymophoma involves chromosome 8, the site of the Myc gene. This changes the pattern of Myc expression, thereboy disrupting its usual function in controlling cell growth and proliferation.

In other embodiments, gene expression associated with colon cancer are identified as the signature component. Two key genes are known to be involved in colon cancer: MSH2 on chromosome 2 and MLH1 on chromosome 3. Normally, the protein products of these genes help to repair mistakes made in DNA replication. If the MSH2 and MLH1 proteins are mutated, the mistakes in replication remain unrepaired, leading to damaged DNA and colon cancer. MEN1 gene, involved in multiple endocrine neoplasia, has been known for several years to be found on chromosome 11, was more finely mapped in 1997, and serves as a signature for such cancers. In preferred embodiments of the present invention, an antibody specific for the altered protein or for the expressed gene to be detected is complexed with DENPs and/or DSNPs of the present invention.

In yet another embodiment, adenocarcinoma of the colon has defined expression of CEA and mutated p53, both well-documented tumor signatures. The mutations of p53 in some of these cell lines are similar to that observed in some of the breast cancer cells and allows for the sharing of a p53 sensing component between the two nanodevices for each of these cancers (i.e., in assembling the DENP, dendrimers comprising the same signature identifying agent may be used for each cancer type). Both colon and breast cancer cells may be reliably studied using cell lines to produce tumors in nude mice, allowing for optimization and characterization in animals.

From the discussion above it is clear that there are many different tumor signatures that find use with the present invention, some of which are specific to a particular type of cancer and others which are promiscuous in their origin. The present invention is not limited to any particular tumor signature or any other disease-specific signature. For example, tumor suppressors that find use as signatures in the present invention include, but are not limited to, p53, Mucl, CEA, p16, p21, p27, CCAM, RB, APC, DCC, NF-1, NF-2, WT-1, MEN-1, MEN-II, p73, VHL, FCC and MCC.

### Biological Imaging Component

In some embodiments of the present invention, the DENP and/or DSNP comprises at least one dendrimer-based nanoscopic building block that can be readily imaged. The present invention is not limited by the nature of the imaging component used. In some embodiments of the present invention, imaging modules comprise surface modifications of quantum dots (See e.g., Chan and Nie, Science 281:2016 (1998)) such as zinc sulfide-capped cadmium selenide coupled to biomolecules (Sooklal, Adv. Mater., 10:1083 (1998)). In some embodiments, the monitoring agent is the metal nanoparticle present within the DENP and/or DSNP.

However, in preferred embodiments, the imaging module comprises dendrimers produced according to the “nanocomposite” concept (Balogh et al., Proc. of ACS PMSE 77:118 (1997) and Balogh and Tomalia, J. Am. Che. Soc., 120:7355 (1998)). In these embodiments, dendrimers are produced by reactive encapsulation, where a reactant is preorganized by the dendrimer template and then subsequently immobilized in/on the polymer molecule by a second reactant (See, e.g., Examples 1-2 and 5-6). Size, shape, size distribution and surface functionality of these nanoparticles are determined and controlled by the dendritic macromolecules. These materials have the solubility and compatibility of the host and have the optical or physiological properties of the guest molecule (i.e., the molecule that permits imaging). While the dendrimer host may vary according to the medium, it is possible to load the dendrimer hosts with different compounds and at various guest concentration levels. Complexes and composites may involve the use of a variety of metals or other inorganic materials. The high electron density of these materials considerably simplifies the imaging by electron microscopy and related scattering techniques. In addition, properties of inorganic atoms introduce new and measurable properties for imaging in either the presence or absence of interfering biological materials. In some embodiments of the present invention, encapsulation of gold, silver, copper, platinum, cobalt, iron atoms/molecules and/or organic dye molecules such as fluorescein are encapsulated into dendrimers for use as nanoscopic composite labels/tracers, although any material that facilitates imaging or detection may be employed. In some embodiment, a DENP and/or DSNP (e.g., a Au DENP and/or DSNP) of the present invention may comprise one or more other imaging agents. For example, in some embodiments, the imaging agent is a fluorescing agent (e.g., fluorescein isothiocyanate).

In some embodiments of the present invention, imaging is based on the passive or active observation of local differences in density of selected physical properties of the investigated complex matter. These differences may be due to a different shape (e.g., mass density detected by atomic force microscopy), altered composition (e.g. radiopaques detected by X-ray), distinct light emission (e.g., fluorochromes detected by spectrophotometry), different diffraction (e.g., electron-beam detected by TEM), contrasted absorption (e.g., light detected by optical methods), or special radiation emission (e.g., isotope methods), etc. Thus, quality and sensitivity of imaging depend on the property observed and on the technique used. The imaging techniques for cancerous cells have to provide sufficient levels of sensitivity to observe small, local concentrations of selected cells. The earliest identification of cancer signatures requires high selectivity (i.e., highly specific recognition provided by appropriate targeting) and the highest possible sensitivity.

### A. Magnetic Resonance Imaging

Once the DENP and/or DSNP has attached to (or been internalized into) a target cell (e.g., a tumor cell or other type of diseased cell or healthy cell), one or more modules of the DENP and/or DSNP (e.g., the metal nanoparticle encapsulated by the dendrimer, and/or, an imaging agent conjugated to the dendrimer) serve to image its location. Dendrimers have already been employed as biomedical imaging agents, perhaps most notably for magnetic resonance imaging (MRI) contrast enhancement agents (See e.g., Wiener et al., Mag. Reson. Med. 31:1 (1994); an example using PAMAM dendrimers). These agents are typically constructed by conjugating chelated paramagnetic ions, such as Gd(III)-diethylenetriaminepentaacetic acid (Gd(III)-DTPA), to water-soluble dendrimers. Other paramagnetic ions that may be useful in this context of the invention include, but are not limited to, gadolinium, manganese, copper, chromium, iron, cobalt, erbium, nickel, europium, technetium, indium, samarium, dysprosium, ruthenium, ytterbium, yttrium, and holmium ions and combinations thereof. In some embodiments of the present invention, the dendrimer is also conjugated to a targeting group, such as epidermal growth factor (EGF), to make the conjugate specifically bind to the desired cell type (e.g., in the case of EGF, EGFR-expressing tumor cells). In a preferred embodiment of the present invention, DTPA is attached to dendrimers via the isothiocyanate of DTPA as described by Wiener (Wiener et al., Mag. Reson. Med. 31:1 (1994)).

DENP and/or DSNP MRI agents are particularly effective due to the polyvalency, size and architecture of DENPs and/or DSNPs (e.g., comprising both dendrimers conjugated to one or more functional groups and an encapsulated metal nanoparticle), which results in molecules with large proton relaxation enhancements, high molecular relaxivity, and a high effective concentration of paramagnetic ions at the target site. Dendrimeric gadolinium contrast agents have even been used to differentiate between benign and malignant breast tumors using dynamic MRI, based on how the vasculature for the latter type of tumor images more densely (Adam et al., Ivest. Rad. 31:26 (1996)). Thus, MRI provides a particularly useful imaging system of the present invention.

### B. Microscopic Imaging

Static structural microscopic imaging of cancerous cells and tissues has traditionally been performed outside of the patient. Classical histology of tissue biopsies provides a fine illustrative example, and has proven a powerful adjunct to cancer diagnosis and treatment. After removal a specimen is sliced thin (e.g., less than 40 microns), stained, fixed, and examined by a pathologist. If images are obtained, they are most often 2-D transmission bright-field projection images. Specialized dyes are employed to provide selective contrast, which is almost absent from the unstained tissue, and to also provide for the identification of aberrant cellular constituents. Quantifying sub-cellular structural features by using computer-assisted analysis, such as in nuclear ploidy determination, is often confounded by the loss of histologic context owing to the thinness of the specimen and the overall lack of 3-D information. Despite the limitations of the static imaging approach, it has been invaluable to allow for the identification of neoplasia in biopsied tissue. Furthermore, its use is often the crucial factor in the decision to perform invasive and risky combinations of chemotherapy, surgical procedures, and radiation treatments, which are often accompanied by severe collateral tissue damage, complications, and even patient death.

The DENPs and/or DSNPs of the present invention allow functional microscopic imaging of tumors and provide improved methods for imaging. The methods find usc iti vivo, in vitro, and ex vivo. For example, in one embodiment of the present invention, DENPs and/or DSNPs of the present invention are designed to emit light or other detectable signals upon exposure to light. Although the labeled DENPs and/or DSNPs may be physically smaller than the optical resolution limit of the microscopy technique, they become self-luminous objects when excited and are readily observable and measurable using optical techniques. In some embodiments of the present invention, sensing fluorescent biosensors in a microscope involves the use of tunable excitation and emission filters and multiwavelength sources (Farkas et al., SPEI 2678:200 (1997)). In embodiments where the imaging agents are present in deeper tissue, longer wavelengths in the Near-infrared (NIR) are used (See e.g., Lester et al., Cell Mol. Biol. 44:29 (1998)). Dendrimeric biosensing in the Near-IR has been demonstrated with dendrimeric biosensing antenna-like architectures (Shortreed et al., J. Phys. Chem., 101:6318 (1997)). Biosensors that find use with the present invention include, but are not limited to, fluorescent dyes and molecular beacons.

In some embodiments of the present invention, in vivo imaging is accomplished using functional imaging techniques. Functional imaging is a complementary and potentially more powerful techniques as compared to static structural imaging. Functional imaging is best known for its application at the macroscopic scale, with examples including functional Magnetic Resonance Imaging (fMRI) and Positron Emission Tomography (PET). However, functional microscopic imaging may also be conducted and find use in in vivo and ex vivo analysis of living tissue. Functional microscopic imaging is an efficient combination of 3-D imaging, 3-D spatial multispectral volumetric assignment, and temporal sampling: in short a type of 3-D spectral microscopic movie loop. Interestingly, cells and tissues autofluoresce. When excited by several wavelengths, providing much of the basic 3-D structure needed to characterize several cellular components (e.g., the nucleus) without specific labeling. Oblique light illumination is also useful to collect structural information and is used routinely. As opposed to structural spectral microimaging, functional spectral microimaging may be used with biosensors, which act to localize physiologic signals within the cell or tissue. For example, in some embodiments of the present invention, biosensor-comprising DENPs and/or DSNPs or DSNPs of the present invention are used to image upregulated receptor families such as the folate or EGF classes. In such embodiments, functional biosensing therefore involves the detection of physiological abnormalities relevant to carcinogenesis or malignancy, even at early stages. A number of physiological conditions may be imaged using the compositions and methods of the present invention including, but not limited to, detection of nanoscopic dendrimeric biosensors for pH, oxygen concentration, Ca²⁺ concentration, and other physiologically relevant analytes.

Other imaging techniques greatly benefited by (e.g., that find great utility for) the compositions and method of the present invention include transmission electron microscopy (TEM) imaging techniques. Such techniques have been widely used to image cell and tissue morphologies using metal nanoparticles as contrasting agents (See, e.g., Li et al., Biomaterials. 17, 3463 (2004); Liu, J Electron Microsc (Tokyo). Epub August 25 (2005)).

Additionally, compositions and methods of the present invention find use in optic and environmental applications. For example, in some embodiments, the compositions and methods of the present invention (e.g., DSNPs and/or DENPs and/or DSNPs) can be used to enhance laser-induced optical breakdown (See, e.g., Ye et al., Applied Physics Letters, 80, 1713 (2002)). In other embodiments, compositions and methods of the present invention are used with fiber-optic sensing techniques to image cell and tissue morphologies and laser-induced optical breakdown (e.g., for cancer treatment). (See, e.g., Ye et al., U.S. Pat. App. No. 20040131322, PCT App. No. WO/2004057386, and Thomas et al., Biophysical Journal, 86(6), 3959 (2004), each of which is herein incorporated by reference in their entireties). In some embodiments, compositions and methods of the present invention (e.g., DSNPs and/or DENPs and/or DSNPs) can be generated and used to remove organic contaminents from waste water (See, e.g., Shi et al., Abstracts of Papers, 229th ACS National Meeting, San Diego, Calif., United States, Mar. 13-17, 2005 (2005), Shi et al., Nanotechnology 2006, 17, 4554-4560).

### Biological Monitoring Component

The biological monitoring or sensing component of the DENPs and/or DSNPs of the present invention is one which that can monitor the particular response in the tumor cell induced by an agent (e.g., a therapeutic agent provided by the therapeutic component of the DENPs and/or DSNPs). While the present invention is not limited to any particular monitoring system, the invention is illustrated by methods and compositions for monitoring cancer treatments. In preferred embodiments of the present invention, the agent induces apoptosis in cells and monitoring involves the detection of apoptosis. In particular embodiments, the monitoring component is an agent that fluoresces at a particular wavelength when apoptosis occurs. For example, in a preferred embodiment, caspase activity activates green fluorescence in the monitoring component. Apoptotic cancer cells, which have turned red as a result of being targeted by a particular signature with a red label, turn orange while residual cancer cells remain red. Normal cells induced to undergo apoptosis (e.g., through collateral damage), if present, will fluoresce green.

In these embodiments, fluorescent groups such as fluorescein are employed in the monitoring component. Fluorescein is easily attached to the dendrimer surface via the isothiocyanate derivatives, available from Molecular Probes, Inc. This allows the nanodevices to be imaged with the cells via confocal microscopy. Sensing of the effectiveness of the nanodevices is preferably achieved by using fluorogenic peptide enzyme substrates. For example, apoptosis caused by the therapeutic agents results in the production of the peptidase caspase-1 (ICE). Calbiochem sells a number of peptide substrates for this enzyme that release a fluorescent moiety. A particularly useful peptide for use in the present invention is:

(SEQ ID NO: 1)

MCA-Tyr-Glu-Val-Asp-Gly-Trp-Lys-(DNP)-NH₂

where MCA is the (7-methoxycoumarin-4-yl)acetyl and DNP is the 2,4-dinitrophenyl group (See, e.g., Talanian et al., J. Biol. Chem., 272: 9677 (1997)). In this peptide, the MCA group has greatly attenuated fluorescence, due to fluorogenic resonance energy transfer (FRET) to the DNP group. When the enzyme cleaves the peptide between the aspartic acid and glycine residues, the MCA and DNP are separated, and the MCA group strongly fluoresces green (excitation maximum at 325 nm and emission maximum at 392 nm).

In preferred embodiments of the present invention, the lysine end of the peptide is linked to the DENPs and/or DSNPs, so that the MCA group is released into the cytosol when it is cleaved. The lysine end of the peptide is a useful synthetic handle for conjugation because, for example, it can react with the activated ester group of a bifunctional linker such as Mal-PEG-OSu. Thus the appearance of green fluorescence in the target cells produced using these methods provides a clear indication that apoptosis has begun (if the cell already has a red color from the presence of aggregated quantum dots, the cell turns orange from the combined colors).

Additional fluorescent dyes that find use with the present invention include, but are not limited to, acridine orange, reported as sensitive to DNA changes in apoptotic cells (Abrams et al., Development 117:29 (1993)) and cis-parinaric acid, sensitive to the lipid peroxidation that accompanies apoptosis (Hockenbery et al., Cell 75:241 (1993)). It should be noted that the peptide and the fluorescent dyes are merely exemplary. It is contemplated that any peptide that effectively acts as a substrate for a caspase produced as a result of apoptosis finds use with the present invention.

### Targeting Components

As described above, another component of the present invention is that the DENP and/or DSNP compositions are able to specifically target a particular cell type (e.g., tumor cell). Although an understanding of the mechanism is not necessary to practice the present invention and the present invention is not limited to any particular mechanism of action, in some embodiments, the DENP and/or DSNP targets a cell (e.g., a neoplastic cell) through a cell surface moiety and is taken into the cell through receptor mediated endocytosis.

Any moiety known to be located on the surface of target cells (e.g. tumor cells or other type of diseased or healthy cell) finds use with the present invention. For example, an antibody directed against such a moiety targets the compositions of the present invention to cell surfaces containing the moiety. Alternatively, the targeting moiety may be a ligand directed to a receptor present on the cell surface or vice versa. In a preferred embodiment of the present invention, the targeting moiety is the folic acid receptor (See, e.g., Examples 4-6). In some embodiments, the targeting moiety is an RGD peptide receptor (e.g., αᵥβ₃ integrin). Similarly, vitamins also may be used to target the therapeutics (e.g., DENPs and/or DSNPs comprising a therapeutic agent) of the present invention to a particular cell.

In some embodiments of the present invention, the targeting moiety may also function as a signatures component. For example, tumor specific antigens including, but not limited to, carcinoembryonic antigen, prostate specific antigen, tyrosinase, ras, a sialyly lewis antigen, erb, MAGE-1, MAGE-3, BAGE, MN, gp100, gp75, p97, proteinase 3, a mucin, CD81, CID9, CD63; CD53, CD38, CO-029, CA125, GD2, GM2 and O-acetyl GD3, M-TA-A, M-fetal or M-urinary find use with the present invention. Alternatively the targeting moiety may be a tumor suppressor, a cytokine, a chemokine, a tumor specific receptor ligand, a receptor, an inducer of apoptosis, or a differentiating agent.

Tumor suppressor proteins contemplated for targeting include, but are not limited to, p16, p21, p27, p53, p73, Rb, Wilns tumor (WT-1), DCC, neurofibromatosis type 1 (NF-1), von Hippel-Lindau (VHL) disease tumor suppressor, Maspin, Brush-1, BRCA-1, BRCA-2, the multiple tumor suppressor (MTS), gp95/p97 antigen of human melanoma, renal cell carcinoma-associated G250 antigen, KS1/4 pan-carcinoma antigen, ovarian carcinoma antigen (CAI 25), prostate specific antigen, melanoma antigen gp75, CD9, CD63, CD53, CD37, R2, CD81, C0029, TI-1, L6 and SAS. Of course these are merely exemplary tumor suppressors and it is envisioned that the present invention may be used in conjunction with any other agent that is or becomes known to those of skill in the art as a tumor suppressor.

In some embodiments of the present invention targeting is directed to factors expressed by an oncogene. These include, but are not limited to, t rosine kinascs, both membrane-associated and cytoplasmic forms, such as members of the Src family, serine/threonine kinases, such as Mos, growth factor and receptors, such as platelet derived growth factor (PDDG), SMALL GTPases (G proteins) including the ras family, cyclin-dependent protein kinases (cdk), members of the myc family members including c-myc, N-myc, and L-myc and bcl-2 and family members.

Cytokines that may be targeted by the present invention include, but are not limited to, IL-1, IL-2, IL-3, IL-4, IL-5, IL-6, IL-7, IL-8, IL-9, IL-10, ILA 1, IL-12, IL-13, IL-14, IL-15, TNF, GMCSF, β-interferon and γ-interferon. Chemokines that may be used include, but are not limited to, M1P1α, M1P1β, and RANTES.

Enzymes that may be targeted by the present invention include, but are not limited to, cytosine deaminase, hypoxanthine-guanine phosphoribosyltransferase, galactose-1-phosphate uridyltransferase, phenylalanine hydroxylase, glucocerbrosidase, sphingomyelinase, alpha-L-iduronidase, glucose-6-phosphate dehydrogenase, HSV thymidine kinase, and human thymidine kinase.

Receptors and their related ligands that find use in the context of the present invention include, but are not limited to, the folate receptor, adrenergic receptor, growth hormone receptor, luteinizing hormone receptor, estrogen receptor, epidermal growth factor receptor, fibroblast growth factor receptor, and the like.

Hormones and their receptors that find use in the targeting aspect of the present invention include, but are not limited to, growth hormone, prolactin, placental lactogen, luteinizing hormone, foilicle-stimulating hormone, chorionic gonadotropin, thyroid-stimulating hormone, leptin, adrenocorticotropin (ACTH), angiotensin I, angiotensin II, .beta.-endorphin, .beta.-melanocyte stimulating hormone (β-MSH), cholecystokinin, endothelin I, galanin, gastric inhibitory peptide (GIP), glucagon, insulin, amylin, lipotropins, GLP-1 (7-37) neurophysins, and somatostatin.

In addition, the present invention contemplates that vitamins (both fat soluble and non-fat soluble vitamins) placed in the targeting component of the nanodevice may be used to target cells that have receptors for, or otherwise take up these vitamins. Particularly preferred for this aspect are the fat soluble vitamins, such as vitamin D and its analogues, vitamin E, Vitamin A, and the like or water soluble vitamins such as Vitamin C, and the like.

In some embodiments of the present invention, any number of cancer cell targeting groups are attached to DENPs and/or DSNPs. Thus a DENP of the present invention is such that it is specific for targeting cancer cells (i.e., much more likely to attach to cancer cells and not to healthy cells). In addition, the polyvalency of the DENPs and/or DSNPs allows the attachment of polyethylene glycol (PEG) or polyethyloxazoline (PEOX) chains to help increase the blood circulation time and decrease the immunogenicity of the DENPs and/or DSNPs.

In preferred embodiments of the present invention, targeting groups are conjugated to DENPs and/or DSNPs with either short (e.g., direct coupling), medium (e.g. using small-molecule bifunctional linkers such as SPDP, sold by Pierce Chemical Company), or long (e.g., PEG bifunctional linkers, sold by Shearwater Polymers) linkages. Since DENPs and/or DSNPs have surfaces with a large number of functional groups (e.g., terminal amine or hydroxyl groups present after functionalization), more than one targeting group may be attached to each dendrimer. As a result, there are multiple binding events between the DENPs and/or DSNPs and the target cell. In these embodiments, the DENPs and/or DSNPs have a very high affinity for their target cells via this “cooperative binding” or polyvalent interaction effect.

For steric reasons, the smaller the ligands, the more can be attached to the surface of a DENPs and/or DSNPs. Recently, Wiener reported that dendrimers with attached folic acid would specifically accumulate on the surface and within tumor cells expressing the high-affinity folate receptor (hFR) (See, e.g., Wiener et al., Invest. Radiol., 32:748 (1997)). The hFR receptor is expressed or upregulated on epithelial tumors, including breast cancers. Control cells lacking hFR showed noi significant accumulation of folate-derivatized dendrimers. Folic acid can be attached to full generation PAMAM dendrimers via a carbodiimide coupling reaction. Folic acid is a good targeting candidate for the dendrimers, with its small size and a simple conjugation procedure.

A larger, yet still relatively small ligand is epidermal growth factor (EGF), a single-chain peptide with 53 amino acid residues. It has been shown that PAMAM dendrimers conjugated to EGF with the linker SPDP bind to the cell surface of human glioma cells and are endocytosed, accumulating in lysosomes (See, e.g., Casale et al., Bioconjugate Chem., 7:7 (1996)). Since EGF receptor density is up to 100 times greater on brain tumor cells compared to normal cells, EGF provides a useful targeting agent for these kinds of tumors. Since the EGF receptor is also overexpressed in breast and colon cancer, EGF may be used as a targeting agent for these cells as well. Similarly, the fibroblast growth factor receptors (EGER) also bind the relatively small polypeptides (FGF), and many are known to be expressed at high levels in breast tumor cell lines (particularly FGF1, 2 and 4) (See, e.g., Penault-Llorca et al., Int. J. Cancer 61:170 (1995)).

In some embodiments of the present invention, the targeting moiety is an antibody or antigen binding fragment of an antibody (e.g., Fab units). For example, a well-studied antigen found on the surface of many cancers (including breast HER2 tumors) is glycoprotein p 185, which is exclusively expressed in malignant cells (See, e.g., Press et al., Oncogene 5:953 (1990)). Recombinant humanized anti-HER2 monoclonal antibodies (rhuMabHER2) have even been shown to inhibit the growth of HER2 overexpressing breast cancer cells, and are being evaluated (in conjunction with conventional chemotherapeutics) in phase III clinical trials for the treatment of advanced breast cancer (See, e.g., Pegram et al., Proc. Am. Soc. Clin. Oncol., 14:106 (1995)). Park and Papahadjopoulos have attached Fab fragments of rhuMabHER2 to small unilamellar liposomes, which then can be loaded with the chemotherapeutic doxorubicin (dox) and targeted to HER2 overexpressing tumor xenografts (See, e.g., Park et al., Cancer Lett., 118:153 (1997) and Kirpotin et al., Biochem., 36:66 (1997)). These dox-loaded “immunoliposomes” showed increased cytotoxicity against tumors compared to corresponding non-targeted dox-loaded liposomes or free dox, and decreased systemic toxicity compared to free dox.

Antibodies can be generated to allow for the targeting of antigens or immunogens (e.g., tumor, tissue or pathogen specific antigens) on various biological targets (e.g., pathogens, tumor cells, normal tissue). Such antibodies include, but are not limited to polyclonal, monoclonal, chimeric, single chain, Fab fragments, and an Fab expression library.

In some embodiments, the antibodies recognize tumor specific epitopes (e.g., TAG-72 (See, e.g., Kjeldsen et al., Cancer Res. 48:2214-2220 (1988); U.S. Pat. Nos. 5,892,020; 5,892,019; and 5,512,443); human carcinoma antigen (See, e.g., U.S. Pat. Nos. 5,693,763; 5,545,530; and 5,808,005); TP1 and TP3 antigens from osteocarcinoma cells (See, e.g., U.S. Pat. No. 5,855,866); Thomsen-Friedenreich (TF) antigen from adenocarcinoma cells (See, e.g., U.S. Pat. No. 5,110,911); “KC-4 antigen” from human prostrate adenocarcinoma (See, e.g., U.S. Pat. Nos. 4,708,930 and 4,743,543); a human colorectal cancer antigen (See, e.g., U.S. Pat. No. 4,921,789); CA125 antigen from cystadenocarcinoma (See, e.g., U.S. Pat. No. 4,921,790); DF3 antigen from human breast carcinoma (See, e.g., U.S. Pat. Nos. 4,963,484 and 5,053,489); a human breast tumor antigen (See, e.g., U.S. Pat. No. 4,939,240); p97 antigen of human melanoma (See, e.g., U.S. Pat. No. 4,918,164); carcinoma or orosomucoid-related antigen (CORA)(See, e.g., U.S. Pat. No. 4,914,021); a human pulmonary carcinoma antigen that reacts with human squamous cell lung carcinoma but not with human small cell lung carcinoma (See, e.g., U.S. Pat. No. 4,892,935); T and Tn haptens in glycoproteins of human breast carcinoma (See, e.g., Springer et al., Carbohydro. Res. 178:271-292 (1988)), MSA breast carcinoma glycoprotein termed (See, e.g., Tjandra et al., Br. J. Surg. 75:811-817 (1988)); MFGM breast carcinoma antigen (See, e.g., Ishida et al., Tumor Biol. 10:12-22 (1989)); DU-PAN-2 pancreatic carcinoma antigen (See, e.g., Lan et al., Cancer Res. 45:305-310 (1985)); CA125 ovarian carcinoma antigen (See, e.g., Hanisch et al., Carbohydr. Res. 178:29-47 (1988)); YH206 lung carcinoma antigen (See, e.g., Hinoda et al., (1988) Cancer J. 42:653-658 (1988)). Each of the foregoing references are specifically incorporated herein by reference.

In other preferred embodiments, the antibodies recognize specific pathogens (e.g., Legionella peomophilia, Mycobacterium tuberculosis, Clostridium tetani, Hemophilus influenzae, Neisseria gonorrhoeae, Treponema pallidum, Bacillus anthracis, Vibrio cholerae, Borrelia burgdorferi, Cornebacterium diphtheria, Staphylococcus aureus, human papilloma virus, human immunodeficiency virus, rubella virus, polio virus, and the like).

Various procedures known in the art are used for the production of polyclonal antibodies. For the production of antibody, various host animals can be immunized by injection with the peptide corresponding to the desired epitope including but not limited to rabbits, mice, rats, sheep, goats, etc. In a preferred embodiment, the peptide is conjugated to an immunogenic carrier (e.g., diphtheria toxoid, bovine serum albumin (BSA), or keyhole limpet hemocyanin (KLH). Various adjuvants are used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (Bacille Calmette-Guerin) and Corynebacterium parvum.

For preparation of monoclonal antibodies, any technique that provides for the production of antibody molecules by continuous cell lines in culture may be used (See e.g., Harlow and Lane, Antibodies: A Laboratory Manual, Cold Spring Harbor Laboratory Press, Cold Spring Harbor, N.Y.). These include, but are not limited to, the hybridoma technique originally developed by Kohler and Milstein (Kohler and Milstein, Nature 256:495-497 (1975)), as well as the trioma technique, the human B-cell hybridoma technique (See e.g., Kozbor et al. Immunol. Today 4:72 (1983)), and the EBV-hybridoma technique to produce human monoclonal antibodies (Cole et al., in Monoclonal Antibodies and Cancer Therapy, Alan R. Liss, Inc., pp. 77-96 (1985)).

In an additional embodiment of the invention, monoclonal antibodies can be produced in germ-free animals utilizing recent technology (See e.g., PCT/US90/02545). According to the invention, human antibodies may be used and can be obtained by using human hybridomas (See, e.g., Cote et al., Proc. Natl. Acad. Sci. U.S.A. 80:2026-2030 (1983)) or by transforming human B cells with EBV virus in vitro (See, e.g., Cole et al., in Monoclonal Antibodies and Cancer Therapy, Alan R. Liss, pp. 77-96 (1985)).

According to the invention, techniques described for the production of single chain antibodies (See, e.g., U.S. Pat. No. 4,946,778; herein incorporated by reference) can be adapted to produce specific single chain antibodies. An additional embodiment of the invention utilizes the techniques described for the construction of Fab expression libraries (See, e.g., Huse et al., Science 246:1275-1281 (1989)) to allow rapid and easy identification of monoclonal Fab fragments with the desired specificity.

Antibody fragments that contain the idiotype (antigen binding region) of the antibody molecule can be generated by known techniques. For example, such fragments include but are not limited to: the F(ab′)2 fragment that can be produced by pepsin digestion of the antibody molecule; the Fab′ fragments that can be generated by reducing the disulfide bridges of the F(ab′)2 fragment, and the Fab fragments that can be generated by treating the antibody molecule with papain and a reducing agent.

In the production of antibodies, screening for the desired antibody can be accomplished by techniques known in the art (e.g., radioimmunoassay, ELISA (enzyme-linked immunosorbant assay), “sandwich” immunoassays, immunoradiometric assays, gel diffusion precipitin reactions, immunodiffusion assays, in situ immunoassays (using colloidal gold, enzyme or radioisotope labels, for example), Western Blots, precipitation reactions, agglutination assays (e.g., gel agglutination assays, hemagglutination assays, etc.), complement fixation assays, immunofluorescence assays, protein A assays, and immunoelectrophoresis assays, etc.).

The DENP systems of the present invention have many advantages over liposomes, such as their greater stability, better control of their size and polydispersity, and generally lower toxicity and immunogenicity (See e.g., Duncan et al, Polymer Preprints 39:180 (1998)). Thus, in some embodiments of the present invention, anti-HER2 antibody fragments, as well as other targeting antibodies are conjugated to DENPs and/or DSNPs, as targeting agents for the nanodevices of the present invention.

In some embodiments, for cancer (e.g., breast cancer), the cell surface may be targeted with folic acid, EGF, FGF, and antibodies (or antibody fragments) to the tumor-associated antigens MUC1, cMet receptor and CD56 (NCAM). Once internalized into the cell, the DENP binds (via conjugated antibodies) to HER2, MUC1 or mutated p53.

The bifunctional linkers SPDP and SMCC and the longer Mal-PEG-OSu linkers are particularly useful for antibody-dendrimer conjugation. In addition, many tumor cells contain surface lectins that bind to oligosaccharides, with specific recognition arising chiefly from the terminal carbohydrate residues of the latter (See, e.g., Sharon and Lis, Science 246:227 (1989)). Attaching appropriate monosaccharides to nonglycosylated proteins such as BSA provides a conjugate that binds to tumor lectin much more tightly than the free monosaccharide (See, e.g., Monsigny et al., Biochemie 70:1633 (1988)).

Mannosylated PAMAM dendrimers bind mannoside-binding lectin up to 400 more avidly than monomeric mannosides (See, e.g., Page and Roy, Bioconjugate Chem., 8:714 (1997)). Sialylated dendrimers and other dendritic polymers bind to and inhibit a variety of sialate-binding viruses both in vitro and in vivo. By conjugating multiple monosaccharide residues (e.g., α-galactoside, for galactose-binding cells) to DENPs and/or DSNPs, polyvalent conjugates are created with a high affinity for the corresponding type of tumor cell. The attachment reaction are easily carried out via reaction of the terminal amines with commercially-available α-galactosidyl-phenylisothiocyanate. The small size of the carbohydrates allows a high concentration to be present on the DENP surface.

A very flexible method to identify and select appropriate peptide targeting groups is the phage display technique (See e.g., Cortese et al., Curr. Opin. Biotechol., 6:73 (1995)), which can be conveniently carried out using commercially available kits. The phage display procedure produces a large and diverse combinatorial library of peptides attached to the surface of phage, which are screened against immobilized surface receptors for tight binding. After the tight-binding, viral constructs are isolated and sequenced to identify the peptide sequences. The cycle is repeated using the best peptides as starting points for the next peptide library. Eventually, suitably high-affinity peptides are identified and then screened for biocompatibility and target specificity. In this way, it is possible to produce peptides that can be conjugated to DENPs and/or DSNPs, producing multivalent conjugates with high specificity and affinity for the target cell receptors (e.g., tumor cell receptors) or other desired targets.

Related to the targeting approaches described above is the “pretargeting” approach (See e.g., Goodwin and Meares, Cancer (suppl.) 80:2675 (1997)). An example of this strategy involves initial treatment of the patient with conjugates of tumor-specific monoclonal antibodies and streptavidin. Remaining soluble conjugate is removed from the bloodstream with an appropriate biotinylated clearing agent. When the tumor-localized conjugate is all that remains, a radiolabeled, biotinylated agent is introduced, which in turn localizes at the tumor sites by the strong and specific biotin-streptavidin interaction. Thus, the radioactive dose is maximized in dose proximity to the cancer cells and minimized in the rest of the body where it can harm healthy cells.

It has been shown that if streptavidin molecules bound to a polystyrene well are first treated with a biotinylated dendrimer, and then radiolabeled streptavidin is introduced, up to four of the labeled streptavidin molecules are bound per polystyrene-bound streptavidin (See, e.g., Wilbur et al., Bioconjugate Chem., 9:813 (1998)). Thus, biotinylated dendrimers (e.g., present within the DENPs and/or DSNPs of the present invention) may be used in the methods of the present invention, acting as a polyvalent receptor for the radiolabel in vivo, with a resulting amplification of the radioactive dosage per bound antibody conjugate. In the preferred embodiments of the present invention, one or more multiply-biotinylated module(s) on the clustered dendrimer presents a polyvalent target for radiolabeled or boronated (See, e.g., Barth et al., Cancer Investigation 14:534 (1996)) avidin or streptavidin, again resulting in an amplified dose of radiation for the tumor cells.

DENPs and/or DSNPs may also be used as clearing agents by, for example, partially biotinylating a dendrimer that has a polyvalent galactose or mannose surface. The conjugate-clearing agent complex would then have a very strong affinity for the corresponding hepatocyte receptors.

In other embodiments of the present invention, an enhanced permeability and retention (EPR) method is used in targeting. The enhanced permeability and retention (EPR) effect is a more “passive” way of targeting tumors (See, e.g., Duncan and Sat, Ann. Oncol., 9:39 (1998)). The EPR effect is the selective concentration of macromolecules and small particles in the tumor microenvironment, caused by the hyperpermeable vasculature and poor lymphatic drainage of tumors. The DENP compositions of the present invention provide ideal polymers for this application, in that they are relatively rigid, of narrow polydispersity, of controlled size and surface chemistry, and have interior “cargo” space that can carry and then release antitumor drugs. In fact, PAMAM dendrimer-platinates have been shown to accumulate in solid tumors (Pt levels about 50 times higher than those obtained with cisplatin) and have in vivo activity in solid tumor models for which cisplatin has no effect (See, e.g., Malik et al., Proc. Int'l. Symp. Control. Rel. Bioact. Mater., 24:107 (1997) and Duncan et al., Polymer Preprints 39:180 (1998)).

The targeting moieties (e.g., DENPs and/or DSNPs comprising one or more targeting agents) of the present invention may recognize a variety of other epitopes on biological targets (e.g., on pathogens). In some embodiments, molecular recognition elements are incorporated to recognize, target or detect a variety of pathogenic organisms including, but not limited to, sialic acid to target HIV (See, e.g., Wies et al., Nature 333: 426 (1988)), influenza (See, e.g., White et al., Cell 56: 725 (1989)), Chlamydia (See, e.g., Infect. Imm. 57: 2378 (1989)), Neisseria meningitidis, Streptococcus suis, Salmonella, mumps, newcastle, and various viruses, including reovirus, Sendai virus, and myxovirus; and 9-OAC sialic acid to target coronavirus, encephalomyelitis virus, and rotavirus; non-sialic acid glycoproteins to detect cytomegalovirus (See, e.g., Virology 176: 337 (1990)) and measles virus (Virology 172: 386 (1989)); CD4 (See, e.g., Khatzman et al., Nature 312: 763 (1985)), vasoactive intestinal peptide (See, e.g., Sacerdote et al., J. of Neuroscience Research 18: 102 (1987)), and peptide T (See, e.g., Ruff et al., FEBS Letters 211: 17 (1987)) to target HIV; epidermal growth factor to target vaccinia (See, e.g., Epstein et al., Nature 318: 663 (1985)); acetylcholine receptor to target rabies (See, e.g., Lentz et al., Science 215: 182 (1982)); Cd3 complement receptor to target Epstein-Barr virus (See, e.g., Carel et al., J. Biol. Chem. 265: 12293 (1990)); beta-adrenergic receptor to target reovirus (See, e.g., Co et al., Proc. Natl. Acad. Sci. 82: 1494 (1985)); ICAM-1 (See, e.g., Marlin et al., Nature 344: 70 (1990)), N-CAM, and myelin-associated glycoprotein MAb (See, e.g., Shephey et al., Proc. Natl. Acad. Sci. 85: 7743 (1988)) to target rhinovirus; polio virus receptor to target polio virus (See, e.g., Mendelsohn et al., Cell 56: 855 (1989)); fibroblast growth factor receptor to target herpes virus (See, e.g., Kaner et al., Science 248: 1410 (1990)); oligomannose to target Escherichia coli; ganglioside G_{M1} to target Neissetia meningitidis; and antibodies to detect a broad variety of pathogens (e.g., Neissetia gonorrhoeae, V. vulnificus, V. parahaemolyticus, V. cholerae, and V. alginolyticus).

In some embodiments of the present invention, the targeting moities are preferably nucleic acids (e.g., RNA or DNA). In some embodiments, the nucleic acid targeting moities are designed to hybridize by base pairing to a particular nucleic acid (e.g., chromosomal DNA, mRNA, or ribosomal RNA). In other embodiments, the nucleic acids bind a ligand or biological target. Nucleic acids that bind the following proteins have been identified: reverse transcriptase, Rev and Tat proteins of HIV (See, e.g., Tuerk et al., Gene 137(1):33-9 (1993)); human nerve growth factor (See, e.g., Binkley et al., Nuc. Acids Res. 23(16):3198-205 (1995)); and vascular endothelial growth factor (See, e.g., Jellinek et al., Biochem. 83(34): 10450-6 (1994)). Nucleic acids that bind ligands are preferably identified by the SELEX procedure (See e.g., U.S. Pat. Nos. 5,475,096; 5,270,163; and 5,475,096; and in PCT publications WO 97/38134, WO 98/33941, and WO 99/07724, all of which are herein incorporated by reference), although many methods are known in the art.

### Evaluation of Anti-Tumor Efficacy and Toxicity of Nanodevice

The anti-tumor effects of various therapeutic agents on cancer cell lines and primary cell cultures may be evaluated using the DENPs and/or DSNPs of the present invention. For example, in preferred embodiments, assays are conducted, ill vitro, using established tumor cell line models or primary culture cells, or alternatively, assays can be conducted in vivo using animal models.

### A. Quantifying the Induction of Apoptosis of Human Tumor Cells In Vitro

In an exemplary embodiment of the present invention, the DENPs and/or DSNPs of the present invention are used to assay apoptosis of human tumor cells in vitro. Testing for apoptosis in the cells determines the efficacy of the therapeutic agent. Multiple aspects of apoptosis can and should be measured. These aspects include those described above, as well as aspects including, but are not limited to, measurement of phosphatidylserine (PS) translocation from the inner to outer surface of plasma membrane, measurement of DNA fragmentation, detection of apoptosis related proteins, and measurement of Caspase-3 activity.

### B. In Vitro Toxicology

In some embodiments of the present invention, to gain a general perspective into the safety of a particular nanodevice platform or component of that system, toxicity testing is performed. Toxicological information may be derived from numerous sources including, but not limited to, historical databases, in vitro testing, and in vivo animal studies.

In vitro toxicological methods have gained popularity in recent years due to increasing desires for alternatives to animal experimentation and an increased perception to the potential ethical, commercial, and scientific value. In vitro toxicity testing systems have numerous advantages including improved efficiency, reduced cost, and reduced variability between experiments. These systems also reduce animal usage, eliminate confounding systemic effects (e.g., immunity), and control environmental conditions.

Although any in vitro testing system may be used with the present invention, the most common approach utilized for in vitro examination is the use of cultured cell models. These systems include freshly isolated cells, primary cells, or transformed cell cultures. Cell culture as the primary means of studying in vitro toxicology is advantageous due to rapid screening of multiple cultures, usefulness in identifying and assessing toxic effects at the cellular, subcellular, or molecular level. In vitro cell culture methods commonly indicate basic cellular toxicity through measurement of membrane integrity, metabolic activities, and subcellular perturbations. Commonly used indicators for membrane integrity include cell viability (cell count), clonal expansion tests, trypan blue exclusion, intracellular enzyme release (e.g. lactate dehydrogenase), membrane permeability of small ions (K¹, Ca²⁺), and intracellular Ala accumulation of small molecules (e.g., ⁵¹Cr, succinate). Subcellular perturbations include monitoring mitochondrial enzyme activity levels via, for example, the MTT test, determining cellular adenine triphosphate (ATP) levels, neutral red uptake into lysosomes, and quantification of total protein synthesis. Metabolic activity indicators include glutathione content, lipid peroxiidation, and lactate/pyruvate ratio.

### C. MTT Assay

The MTT assay is a fast, accurate, and reliable methodology for obtaining cell viability measurements. The MTT assay was first developed by Mosmann (See, e.g., Mosmann, J. Immunol. Meth., 65:55 (1983)). It is a simple colorimetric assay numerous laboratories have utilized for obtaining toxicity results (See e.g., Kuhlmann et al., Arch. Toxicol., 72:536 (1998)). Briefly, the mitochondria produce ATP to provide sufficient energy for the cell. In order to do this, the mitochondria metabolize pyruvate to produce acetyl CoA. Within the mitochondria, acetyl CoA reacts with various enzymes in the tricarboxylic acid cycle resulting in subsequent production of ATP. One of the enzymes particularly useful in the MTT assay is succinate dehydrogenase. MTT (3-(4,5-dimethylthiazol-2-yl)-2 diphenyl tetrazolium bromide) is a yellow substrate that is cleaved by succinate dehydrogenase forming a purple formazan product. The alteration in pigment identifies changes in mitochondria function. Nonviable cells are unable to produce formazan, and therefore, the amount produced directly correlates to the quantity of viable cells. Absorbance at 540 nr is utilized to measure the amount of formazan product.

The results of the in vitro tests can be compared to in vivo toxicity tests in order to extrapolate to live animal conditions. Typically, acute toxicity from a single dose of the substance is assessed. Animals are monitored over 14 days for any signs of toxicity (increased temperature, breathing difficulty, death, etc). Traditionally, the standard of acute toxicity is the median lethal dose (LD₅₀), which is the predicted dose at which half of the treated population would be killed. The determination of this dose occurs by exposing test animals to a geometric series of doses under controlled conditions. Other tests include subacute toxicity testing, which measures the animal's response to repeated doses of the nanodevice for no longer than 14 days. Subchronic toxicity testing involves testing of a repeated dose for 90 days. Chronic toxicity testing is similar to subchronic testing but may last for over a 90-day period. In vivo testing can also be conducted to determine toxicity with respect to certain tissues. For example, in some embodiments of the present invention tumor toxicity (i.e., effect of the compositions of the present invention on the survival of tumor tissue) is determined (e.g., by detecting changes in the size and/or growth of tumor tissues).

### Gene Therapy Vectors

In particular embodiments of the present invention, the DENP and/or DSNP compositions comprise transgenes for delivery and expression to a target cell or tissue, in vitro, ex vivo, or in vivo. In such embodiments, rather than containing the actual protein, the DENP and/or DSNP complex comprises an expression vector construct containing, for example, a heterologous DNA encoding a gene of interest and the various regulatory elements that facilitate the production of the particular protein of interest in the target cells.

In some embodiments, the gene is a therapeutic gene that is used, for example, to treat cancer, to replace a defective gene, or a marker or reporter gene that is used for selection or monitoring purposes. In the context of a gene therapy vector, the gene may be a heterologous piece of DNA. The heterologous DNA may be derived from more than one source (i.e., a multigene construct or a fusion protein). Further, the heterologous DNA may include a regulatory sequence derived from one source and the gene derived from a different source.

Tissue-specific promoters may be used to effect transcription in specific tissues or cells so as to reduce potential toxicity or undesirable effects to non-targeted tissues. For example, promoters such as the PSA, probasin, prostatic acid phosphatase or prostate-specific glandular kallikrein (hK2) may be used to target gene expression in the prostate. Similarly, promoters may be used to target gene expression in other tissues (e.g., insulin, elastin amylase, pdr-1, pdx-1 and glucokinase promoters target to the pancreas; albumin PEPCK, HBV enhancer, alpha fetoproteinapolipoprotein C, alpha-1 antitrypsin, vitellogenin, NF-AB and transthyretin promoters target to the liver; myosin H chain, muscle creatine kinase, dystrophin, calpain p94, skeletal alpha-actin, fast troponin 1 promoters target to skeletal muscle; keratin promoters target the skin; sm22 alpha; SM-.alpha.-actin promoters target smooth muscle; CFTR; human cytokeratin 18 (K18); pulmonary surfactant proteins A, B and Q CC-10; P1 promoters target lung tissue; endothelin-1; E-selectin; von Willebrand factor; KDR/flk-1 target the endothelium; tyrosinase targets melanocytes).

The nucleic acid may be either cDNA or genomic DNA. The nucleic acid can encode any suitable therapeutic protein. Preferably, the nucleic acid encodes a tumor suppressor, cytokine, receptor, inducer of apoptosis, or differentiating agent. The nucleic acid may be an antisense nucleic acid. In such embodiments, the antisense nucleic acid may be incorporated into the DENPs and/or DSNPs of the present invention outside of the context of an expression vector.

In preferred embodiments, the nucleic acid encodes a tumor suppressor, cytokines, receptors, or inducers of apoptosis. Suitable tumor suppressors include BRCA 1, BRCA2, C-CAM, p16, p211 p53, p73, or Rb. Suitable cytokines include GMCSF, IL-1, IL-2, IL-3, IL-4, IL-5, IL6, IL-7, IL-8, IL-9, IL-10, IL-11, IL-12, IL-13, IL-14, IL-15, β-interferon, γ-interferon, or TNF. Suitable receptors include CFTR, EGFR, estrogen receptor, IL-2 receptor, or VEGFR. Suitable inducers of apoptosis include AdE1B, Bad, Bak, Bax, Bid, Bik, Bim, Harakiri, or ICE-CED3 protease.

### Methods of Combined Therapy

Tumor cell resistance to DNA damaging agents represents a major problem in clinical oncology. The DENPs and/or DSNPs of the present invention provide means of ameliorating this problem by effectively administering a combined therapy approach. However, it should be noted that traditional combination therapy may be employed in combination with the DENPs and/or DSNPs of the present invention. For example, in some embodiments of the present invention, DENPs and/or DSNPs may be used before, after, or in combination with the traditional therapies.

To kill cells, inhibit cell growth, or metastasis, or angiogenesis, or otherwise reverse or reduce the malignant phenotype of tumor cells using the methods and compositions of the present invention in combination therapy, one contacts a “target” cell with the DENP compositions described herein and at least one other agent. These compositions are provided in a combined amount effective to kill or inhibit proliferation of the cell. This process may involve contacting the cells with the immunotherapeutic agent and the agent(s) or factor(s) at the same time. This may be achieved by contacting the cell with a single composition or pharmacological formulation that includes both agents, or by contacting the cell with two distinct compositions or formulations, at the same time, wherein one composition includes, for example, an expression construct and the other includes a therapeutic agent.

Alternatively, the DENP and/or DSNP treatment may precede or follow the other agent treatment by intervals ranging from minutes to weeks. In embodiments where the other agent and immunotherapy are applied separately to the cell, one would generally ensure that a significant period of time did not expire between the time of each delivery, such that the agent and DENP and/or DSNP would still be able to exert an advantageously combined effect on the cell. In such instances, it is contemplated that cells are contacted with both modalities within about 12-24 hours of each other and, more preferably, within about 6-12 hours of each other, with a delay time of only about 12 hours being most preferred. In some situations, it may be desirable to extend the time period for treatment significantly, however, where several days (2 to 7) to several weeks (1 to 8) lapse between the respective administrations.

In some embodiments, more than one administration of the immunotherapeutic composition of the present invention or the other agent are utilized. Various combinations may be employed, where the DENP and/or DSNP is “A” and the other agent is “B”, as exemplified below:

### A/B/A, B/A/B, B/B/A, A/A/B, B/A/A, A/B/B, B/B/B/A, B/B/A/B,

### A/A/B/B, A/B/A/B, A/B/B/A, B/B/A/A, B/A/B/A, B/A/A/B, B/B/B/A,

### A/A/A/B, B/A/A/A, A/B/A/A, A/A/B/A, A/B/B/B, B/A/B/B, B/B/A/B.

Other combinations are contemplated. Again, to achieve cell killing, both agents are delivered to a cell in a combined amount effective to kill or disable the cell.

Other factors that may be used in combination therapy with the DENPs and/or DSNPs of the present invention include, but are not limited to, factors that cause DNA damage such as γ-rays, X-rays, and/or the directed delivery of radioisotopes to tumor cells. Other forms of DNA damaging factors are also contemplated such as microwaves and UV-irradiation. Dosage ranges for X-rays range from daily doses of 50 to 200 roentgens for prolonged periods of time (3 to 4 weeks), to single doses of 2000 to 6000 roentgens. Dosage ranges for radioisotopes vary widely, and depend on the half-life of the isotope, the strength and type of radiation emitted, and the uptake by the neoplastic cells. The skilled artisan is directed to “Remington's Pharmaceutical Sciences” 15th Edition, chapter 33, in particular pages 624-652. Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

In preferred embodiments of the present invention, the regional delivery of the nanodevice to patients with cancers is utilized to maximize the therapeutic effectiveness of the delivered agent. Similarly, a DENP comprising one or more functional groups (e.g., a therapeutic agent such as a chemotherapeutic or radiotherapeutic) may be directed to particular, affected region of the subjects body. Alternatively, systemic delivery of a DENP and/or DSNP (e.g., a DENP and/or DSNP comprising a therapeutic agent, targeting agent, and/or imaging agent) may be appropriate in certain circumstances, for example, where extensive metastasis has occurred, or where metastasis is suspected.

In addition to combining the DENPs and/or DSNPs with chemo- and radiotherapies, it also is contemplated that traditional gene therapies are used. For example, targeting of p53 or p16 mutations along with treatment of the DENP and/or DSNP provides an improved anti-cancer treatment. The present invention contemplates the co-treatment with other tumor-related genes including, but not limited to, p21, Rb, APC, DCC, NF-I, NF-2, BCRA2, p16, FHIT, WT-I, MEN-I, MEN-II, BRCA1, VHL, FCC, MCC, ras, myc, neu, raf erb, src, fms, jun, trk, ret, gsp, hst, bcl, and abl.

In vivo and ex vivo treatments are applied using the appropriate methods worked out for the gene delivery of a particular construct for a particular subject. For example, for viral vectors, one typically delivers 1×10⁴, 1×10⁵, 1×10⁶, 1×10⁷, 1×10⁸, 1×10⁹, 1×10¹⁰, 1×10¹¹ or 1×10¹² infectious particles to the patient. Similar figures may be extrapolated for liposomal or other non-viral formulations by comparing relative uptake efficiencies.

An attractive feature of the present invention is that the therapeutic compositions comprising DENPs and/or DSNPs may be delivered to local sites in a patient by a medical device. Medical devices that are suitable for use in the present invention include known devices for the localized delivery of therapeutic agents. Such devices include, but are not limited to, catheters such as injection catheters, balloon catheters, double balloon catheters, microporous balloon catheters, channel balloon catheters, infusion catheters, perfusion catheters, etc., which are, for example, coated with the therapeutic agents or through which the DENPs and/or DSNPs are administered; needle injection devices such as hypodermic needles and needle injection catheters; needleless injection devices such as jet injectors; coated stents, bifurcated stents, vascular grafts, stent grafts, etc.; and coated vaso-occlusive devices such as wire coils.

Exemplary devices are described in U.S. Pat. Nos. 5,935,114; 5,908,413; 5,792,105; 5,693,014; 5,674,192; 5,876,445; 5,913,894; 5,868,719; 5,851,228; 5,843,089; 5,800,519; 5,800,508; 5,800,391; 5,354,308; 5,755,722; 5,733,303; 5,866,561; 5,857,998; 5,843,003; and 5,933,145; the entire contents of which are incorporated herein by reference. Exemplary stents that are commercially available and may be used in the present application include the RADIUS (Scimed Life Systems, Inc.), the SYMPHONY (Boston Scientific Corporation), the Wallstent (Schneider Inc.), the PRECEDENT II (Boston Scientific Corporation) and the NIR (Medinol Inc.). Such devices are delivered to and/or implanted at target locations within the body by known techniques.

### Photodynamic Therapy

In some embodiments, the DENPs and/or DSNPs of the present invention comprise a photodynamic compound and a targeting agent that is administered to a patient. In some embodiments, the targeting agent is then allowed a period of time to bind the “target” cell (e.g. about 1 minute to 24 hours) resulting in the formation of a target cell-target agent complex. In some embodiments, the DENPs and/or DSNPs comprising the targeting agent and photodynamic compound are then illuminated (e.g., with a red laser, incandescent lamp, X-rays, or filtered sunlight). In some embodiments, the light is aimed at the jugular vein or some other superficial blood or lymphatic vessel. In some embodiments, the singlet oxygen and free radicals diffuse from the photodynamic compound to the target cell (e.g. cancer cell or pathogen) causing its destruction.

### Pharmaceutical Formulations

Where clinical applications are contemplated, in some embodiments of the present invention, the DENPs and/or DSNPs are prepared as part of a pharmaceutical composition in a form appropriate for the intended application. Generally, this entails preparing compositions that are essentially free of pyrogens, as well as other impurities that could be harmful to humans or animals. However, in some embodiments of the present invention, a straight DENP formulation may be administered using one or more of the routes described herein.

In preferred embodiments, the DENPs and/or DSNPs are used in conjunction with appropriate salts and buffers to render delivery of the compositions in a stable manner to allow for uptake by target cells. Buffers also are employed when the DENPs and/or DSNPs are introduced into a patient. Aqueous compositions comprise an effective amount of the DENPs and/or DSNPs to cells dispersed in a pharmaceutically acceptable carrier or aqueous medium. Such compositions also are referred to as inocula. The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. Except insofar as any conventional media or agent is incompatible with the vectors or cells of the present invention, its use in therapeutic compositions is contemplated. Supplementary active ingredients may also be incorporated into the compositions.

In some embodiments of the present invention, the active compositions include classic pharmaceutical preparations. Administration of these compositions according to the present invention is via any common route so long as the target tissue is available via that route. This includes oral, nasal, buccal, rectal, vaginal or topical. Alternatively, administration may be by orthotopic, intradermal, subcutaneous, intramuscular, intraperitoneal or intravenous injection.

The active DENPs and/or DSNPs may also be administered parenterally or intraperitoneally or intratumorally. Solutions of the active compounds as free base or pharmacologically acceptable salts are prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

In some embodiments, the present invention provides a composition comprising a DENP and/or DSNP comprising a targeting agent, a therapeutic agent and an imaging agent. In preferred embodiments, the dendrimer is used for delivery of a therapeutic agent (e.g., methotrexate) to tumor cells in vivo. In some embodiments, the therapeutic agent is conjugated to the DENP and/or DSNP via an acid-labile linker. Thus, in some embodiments, the therapeutic agent is released from the dendrimer within a target cell (e.g., within an endosome). This type of intracellular release (e.g., endosomal disruption of the acid-labile linker) is contemplated to provide additional specificity for the compositions and methods of the present invention. In preferred embodiments, the DENPs and/or DSNPs of the present invention contain between 100-150 primary amines on the surface of the dendrimer. Thus, the present invention provides DENPs and/or DSNPs with multiple (e.g., 100-150) reactive sites for the conjugation of functional groups comprising, but not limited to, therapeutic agents, targeting agents, imaging agents and biological monitoring agents, or for functionalizing (e.g., adding a hydroxyl or acetamide group to), thereby making the DENP less toxic.

The compositions and methods of the present invention are contemplated to be equally effective whether or not the DENP and/or DSNP compositions of the present invention comprise a fluorescein (e.g. FITC) imaging agent. Thus, each functional group present in a DENP and/or DSNP composition is able to work independently of the other functional groups. Thus, the present invention provides a dendrimer (e.g., that is part of the DENP and/or DSNP) that can comprise multiple combinations of targeting, therapeutic, imaging, and biological monitoring functional groups.

The present invention also provides a very effective and specific method of delivering molecules (e.g., therapeutic and imaging functional groups) to the interior of target cells (e.g., cancer cells). Thus, in some embodiments, the present invention provides methods of therapy that comprise or require delivery of molecules into a cell in order to function (e.g., delivery of genetic material such as siRNAs).

In some embodiments, pharmaceutical forms suitable for injectable use include sterile aqueous solutions or dispersions and sterile powders for the extemporaneous preparation of sterile injectable solutions or dispersions. The carrier may be a solvent or dispersion medium containing, for example, water, etnanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial an antifungal agents, for example, parabens, chlorobutanol, phenol, sorbic acid, thimerosal, and the like. In many cases, it may be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Upon formulation, the dendrimer compositions are administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution is suitably buffered, if necessary, and the liquid diluent first rendered isotonic with sufficient saline or glucose. These particular aqueous solutions are especially suitable for intravenous, intramuscular, subcutaneous and intraperitoneal administration. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). In some embodiments of the present invention, the DENPs and/or DSNPs are formulated within a therapeutic mixture to comprise about 0.0001 to 1.0 milligrams, or about 0.001 to 0.1 milligrams, or about 0.1 to 1.0 or even about 10 milligrams per dose or so. Multiple doses may be administered.

Additional formulations that are suitable for other modes of administration include vaginal suppositories and pessaries. A rectal pessary or suppository may also be used. Suppositories are solid dosage forms of various weights and shapes, usually medicated, for insertion into the rectum, vagina or the urethra. After insertion, suppositories soften, melt or dissolve in the cavity fluids. In general, for suppositories, traditional binders and carriers may include, for example, polyalkylene glycols or triglycerides; such suppositories may be formed from mixtures containing the active ingredient in the range of 0.5% to 10%, preferably 1%-2%. Vaginal suppositories or pessaries are usually globular or oviform and weighing about 5 g each. Vaginal medications are available in a variety of physical forms, e.g., creams, gels or liquids, which depart from the classical concept of suppositories. In addition, suppositories may be used in connection with colon cancer. The DENPs and/or DSNPs also may be formulated as inhalants for the treatment of lung cancer and such like.

### Method of Treatment or Prevention of Cancer and Pathogenic Diseases

In specific embodiments of the present invention methods and compositions are provided for the treatment of tumors in cancer therapy. It is contemplated that the present therapy can be employed in the treatment of any cancer for which a specific signature has been identified or which can be targeted. Cell proliferative disorders, or cancers, contemplated to be treatable with compositions and methods of the present invention include, but are not limited to, human sarcomas and carcinomas, including, but not limited to, fibrosarcoma, myxosarcoma, liposarcoma, chondrosarcoma, osteogenic sarcoma, chordoma, angiosarcoma, endotheliosarcoma, lymphangiosarcoma, Ewing's tumor, lymphangioendotheliosarcoma, synovioma, mesothelioma, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilns' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, acute lymphocytic leukemia and acute myelocytic leukemia (myeloblastic, promyelocytic, myelomonocytic, monocytic and erythroleukemia); chronic leukemia (chronic myclocytic (granulocytic) leukemia and chronic lymphocytic leukemia); and polycythemia vera, lymphoma (Hodgkin's disease and non-Hodgkin's disease), multiple myeloma, Waldenstrbm's macroglobulinemia, and heavy chain disease.

It is contemplated that the present therapy can be employed in the treatment of any pathogenic disease for which a specific signature has been identified or which can be targeted for a given pathogen. Examples of pathogens contemplated to be treatable with the methods of the present invention include, but are not limited to, Legionella peomophilia, Mycobacterium tuberculosis, Clostridium tetani, Hemophilus influenzae, Neisseria gonorrhoeae, Treponema pallidum, Bacillus anthracis, Vibrio cholerae, Borrelia burgdorferi, Cornebacterium diphtheria, Staphylococcus aureus, human papilloma virus, human immunodeficiency virus, rubella virus, polio virus, and the like.

### EXPERIMENTAL

The following examples are provided in order to demonstrate and fuarthcr illustrate certain preferred embodiments and aspects of the present invention and are not to be construed as limiting the scope thereof.

In the experimental disclosure which follows, the following abbreviations apply: g (grams); 1 or L (liters); μg (micrograms); μl (microliters); μm (micrometers); μM (micromolar); μmol (micromoles); mg (milligrams); ml (milliliters); mm (millimeters); mM (millimolar); mmol (millimoles); M (molar); mol (moles); ng (nanograms); nm (nanometers); nmol (nanomoles); N (normal); and pmol (picomoles).

### Example 1

### Crystalline Dendrimer-Stabilized Gold Nanoparticles

Materials. Amine-terminated PAMAM dendrimers of generation 2 through 6 (E2.NH₂ through E6.NH₂, E denotes the ethylenediamine core) were purchased from Dendritech (Midland, Mich.). All other chemicals were obtained from Aldrich and used as received. Water used in all of the experiments was purified using a Milli-Q Plus 185 water purification system (Millipore, Bedford, Mass., USA) with resistivity higher than 18 MΩ.cm. Regenerated cellulose dialysis membranes (MWCO=10,000) were acquired from Fisher. The Au DSNPs were prepared using hydrazine reduction chemistry in the same dendrimer terminal amine (DTA)/gold atom molar ratios according to a described procedure (See, e.g., Balogh et al., J. Nanoparticle Res. 1999, 1, 353-368). They are denoted as {(Au⁰)₆-E2 NH₂}, {(Au⁰)₁₂-E3.NH₂}, {(Au⁰)₂₄-E4.NH₂}, {(Au⁰)₅₇-E5.NH₂}, and {(Au⁰)₉₈-E6.NH₂}, respectively. The preparation stoichiometry is shown in Table 1, below.

TABLE 1
Preparation parameters and zeta potentials of Au DSNPs.

| | | | | | | Au:D | DTA^{[b]}/Au | |
|---|---|---|---|---|---|---|---|---|
| Au DSNPs | D^{[a]} | C_{D}(mg/mL) | D (mol) | Au (mol) | C_{Au}(mg/mL) | molar ratio | Molar ratio | Zeta potential (mV) |
| {(Au⁰)₆-E2•NH₂} | E2•NH₂ | 10.82 | 1.6610e−5 | 9.95e−5 | 0.003940 | 5.99:1 | 2.67:1 | +39.67 |
| {(Au⁰)₁₂-E3•NH₂} | E3•NH₂ | 11.29 | 8.1710e−6 | 9.95e−5 | 0.003940 | 12.18:1 | 2.63:1 | +26.42 |
| {(Au⁰)₂₄-E4•NH₂} | E4•NH₂ | 11.90 | 4.1860e−6 | 9.95e−5 | 0.003940 | 23.78:1 | 2.69:1 | +39.98 |
| {(Au⁰)₅₇-E5•NH₂} | E5•NH₂ | 10.14 | 1.7580e−6 | 9.95e−5 | 0.003940 | 56.61:1 | 2.26:1 | +41.11 |
| {(Au⁰)₉₃-E6•NH₂} | E6•NH₂ | 11.77 | 1.0140e−6 | 9.95e−5 | 0.003940 | 98.15:1 | 2.61:1 | +40.21 |

^{[a]}D denotes dendrimer;
^{[b]}DTA denotes dendrimer terminal amines.

Briefly, gold-dendrimer complexes were prepared in aqueous solution by mixing 5 mL of 20 mM aqueous solutions of HAuCl₄ with 5 mL aqueous solutions of the respective PAMAM dendrimers with identical molar ratios of DTA/Au atoms. The yellow HAuCl₄ solution lost its color immediately upon mixing with the PAMAMs indicating the formation of complexes between the dendrimer terminal amines and the gold anions. Stable Au DSNPs were prepared by reducing the PAMAM-tetrachloroaurate complexes at room temperature with 50 mol % excess of hydrazine under vigorous magnetic stirring for 2 h. Upon addition of the hydrazine solution to the PAMAM-tetrachloroaurate complexes, a color change from slightly yellow to deep red indicated the formation of zerovalent gold.

Ultraviolet-visible spectrometry. Ultraviolet-visible spectra were collected using a Lambda 20 UV-Vis Spectrometer. All samples were dissolved in water at the concentration of 0.1 mg/mL.

Fluorescence spectroscopy. The excitation and emission spectra of Au DSNPs were collected using the Fluoromax-2 Fluorimeter. All samples were dissolved in water at the concentration of 0.1 mg/mL. The excitation spectra were collected at the range of 230-440 nm with an emission wavelength at 450 nm. The emission spectra were collected at the range of 410-780 nm with an excitation wavelength at 400 nm. Both excitation and emission slit openings were set as 5 nm.

Zeta Potential measurements. Zeta Potential measurements were performed using a PSS/NICOMP 380 ZLS particle sizing system (Santa Barbara, Calif.) with a red-diode laser at 635 nm in a multiangle cell.

Transmission electron microscopy (TEM). A JEOL 2010F Analytical Electron Microscope was performed at 200 kV with an EDS system attached. 5 μL aqueous solution of Au DSNPs (0.1 mg/mL) was dropped onto carbon-coated copper grid and air dried before measurements. Ultrathin sections of the PAGE gel samples of Au-DSNPs for TEM were sliced with a Leica ultracut UCT ultramicrotome after setting them in LR-white resin. The thin sections were placed onto carbon-coated copper grids.

Polyacrylamide gel electrophoresis (PAGE). Analysis of PAMAM dendrimers and Au DSNPs by PAGE was performed on a Micrograd vertical electrophoresis system (Model FB-VE10-1, FisherBiotech, Pittsburgh, Pa.) with a commercial power supply (Model EC135-90; Thermo Electron Corporation, Milford, Mass.). Precast 4-20% gradient express gels for PAGE were obtained from ISC BioExpress (Kaysville, Utah). Tris-Glycine (TG) native buffer (pH=8.3) was purchased from Invitrogen (Carlsbad, Calif.). It was diluted by a factor of ten to prepare the running buffer. PAGE separations typically required 50 min at 200V. Reverse polarity was used for the analysis of the polycationic PAMAM dendrimers and Au DSNPs. Into each sample well 2 μL of a sample solution composed of 1 μL 1 mg/mL PAMAM dendrimer or Au DSNPs and 1 μL methylene blue sucrose dye solutions (50% sucrose, 1% methylene blue) was injected. Developed gels were stained with 0.025% Comassie Blue R-250 in 40% methanol and 7% acetic acid aqueous solution overnight. The gels were destained with 7% (v/v) acetic acid and 5% (v/v) methanol in water.

Generation of crystalline dendrimer-stabilized gold nanoparticles. Primary amine-terminated PAMAMs of generation 2 through 6 were selected to prepare Au DSNPs with consistent molar ratios of dendrimer terminal amines and gold atoms. Various characterization techniques were employed to investigate their structural characteristics including UV-Vis spectrometry, fluorescence spectroscopy, transmission electron microscopy (TEM), zeta potential measurements, and polyacrylamide gel electrophoresis (PAGE). UV-Vis and fluorescence spectrometry disclose the optical properties of Au DSNPs of some embodiments of the present invention, while electron microscopy imaging and selected area electron diffraction (SAED) are able to characterize the morphology, sizes, and crystal structure of Au DSNPs. Zeta potential measurements were used to record the surface charge potentials of the formed Au DSNPs. The stability of Au DSNPs was further characterized using both PAGE. In some embodiments, characterization of the Au DSNPs provides an understanding of the structures and properties of Au DSNPs, the understanding of which can then be used to in post-modification schemes. Such post modification schemes are useful for post-modifying the Au DSNPs with various biological ligands (e.g., functional groups including, but not limited to biological sensing groups, targeting groups, and therapeutic groups) and treatment of cells (e.g., cancer cells or infected cells) in vitro and in vivo.

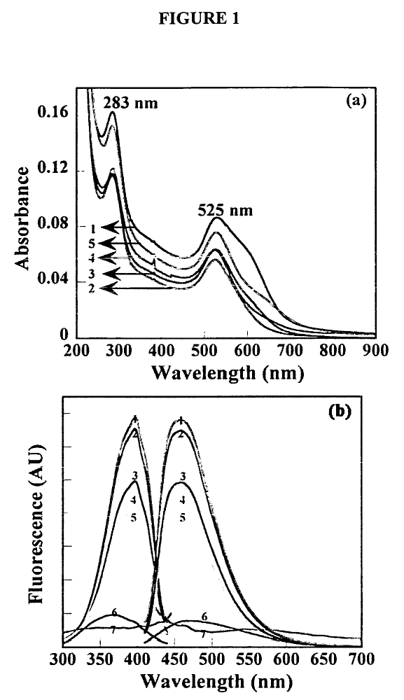

The stoichiometry used to prepare Au DSNPs of some embodiments of the present invention is listed in Table 1. For Au-DSNPs, the molar ratio between dendrimer terminal amines (DTA) and Au atoms are consistent except that there is some variation for E5.NH₂ PAMAM dendrimer (E denotes ethylenediamine core, 5 is the generation number). FIG. 1a shows the UV-Vis spectra of Au DSNPs prepared using PAMAMs of generation 2 through 6. The plasmon peak at around 525 nm is clearly observed for all samples, which can be attributed to the transition between the 5d¹⁰ level and unoccupied conduction bands of gold NPs (See, e.g., Alvarez et al., J. Phys. Chem. B 1997, 101, 3703). The larger size of {(Au⁰)₆-E2.NH₂} DSNPs was also confirmed by TEM measurements as described below. The absorbance peak at 283 nm for all Au DSNPs is assigned to certain carbonyl compounds formed presumably by oxidation of the dendrimers (Esumi et al., Langmuir, 14, 3157-3159 (1998)). FIG. 1b shows the fluorescence spectra of Au DSNPs and commercial Au colloid particles (5 nm and 100 nm). Au DSNPs were found to be fluorescent and display strong emissions. The maximum excitation and emission wavelengths were around 397 nm and 458 nm, respectively, in agreement with other studies (See, e.g., Zheng et al., J. Am. Chem. Soc. 2003, 125, 7780-7781). In contrast, commercial gold colloids (5 nm and 100 nm) that are prepared using citric acid reduction and protection approach do not exhibit fluorescence emission, suggesting that the dendrimer stabilizers play an important role or contribute to the fluorescence properties of the formed Au DSNPs. Both PAMAM and polypropyleneimine (PPI) dendrimers exhibit strong intrinsic fluorescence emission at certain concentration ranges (See, e.g., Wang and Imae, J. Am. Chem. Soc. 2004, 126, 13204-13205). It has been proposed that the backbone of dendrimers plays a key role in forming the fluorescence center. The fluorescent properties of the formed Au DSNPs provide potentially useful fluorescent markers for cell labeling and biological sensing studies.

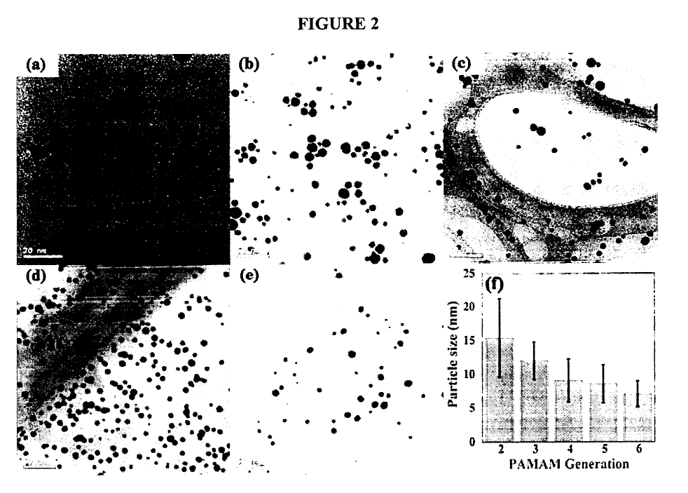

The size distribution and morphology of the synthesized Au DSNPs were studied by TEM. FIG. 2 shows TEM images of Au DSNPs prepared using PAMAM dendrimers of different generations. The sizes of the formed Au DSNPs are 15.4±5.8 nm, 12.0±2.8 nm, 9.1±3.2 nm, 8.6±2.8 nm, and 7.1±1.9 nm, for {(Au⁰)₆-E2.NH₂}, {(Au⁰)₁₂-E3.NH₂}, {(Au⁰)₂₄-E4.NH₂}, {(Au⁰)₅₇-E5.NH₂}, and {(Au⁰)₉₈-E6.NH₂}, respectively. All of the Au DSNPs are relatively monodispersed except {(Au⁰)₆-E2.NH₂}. {(Au⁰)₆-E2.NH₂} displays larger size and higher polydispersity, which can be attributed to limited number of amines of E2.NH₂ dendrimer to stabilize or encapsulate Au NPs.

Although an understanding of the mechanism is not necessary to practice the present invention and the present invention is not limited to any particular mechanism of action, the size of the Au DSNPs decreases with the increase of the number of dendrimer generations (See FIG. 2f), suggesting that there exist different nucleation and growth mechanisms for gold nanocrystals in the presence of PAMAM dendrimers. At basic pH conditions (pH≈10.4 when dendrimers are dissolved in water), AuCl₄⁻ anions bind preferably to the protonated amines of PAMAM dendrimers through electrostatic interaction. Larger generation PAMAM dendrimers have denser structures that would significantly limit the nucleation, movement, and growth of gold nanocrystals. In contrast, smaller generation PAMAMs have relatively open structures, that hinder the growth of gold nanocrystals less significantly than larger generation PAMAMs. Thus, in some embodiments, individual Au NPs may be stabilized by several dendrimer molecules. In some embodiments, the synthesized Au DSNPs of the present invention are significantly larger than those reported in the literature (See, e.g., Grohn et al, Macromolecules 2000, 33, 6042-6050; Manna et al., Chem. Mater. 2001, 13, 1674-1681; Esumi et al., Langmuir 2000, 16, 2604-2608; Kim et al., Chem. Mater. 2004, 16, 167-172).

Those of skill in the art appreciated that nanoparticle sizes and morphologies are variable under different preparation conditions (e.g., selection of reduction reagents, concentration, temperature, and solvent systems, See Cushing et al., Chem. Rev. 2004, 104, 3893-3946). Although an understanding of the mechanism is not necessary to practice the present invention and the present invention is not limited to any particular mechanism of action, it is believed that fast reduction induces the formation of smaller NPs, while slow reduction favors the aggregation and Ostwald ripening of NPs (See, e.g., Hayakawa et al., Langmuir 2003, 19, 5517-5521). The reduction potential of NH₂═NH₂ (−0.09 V) used in the present invention is significantly smaller than NaBH₄ (−0.481 V) which has been used by others (See, e.g., Dean, Lange's Handbook of Chemistry, 14th Edition ed.; MaGRAW-HILL, Inc: New York, 1992; Lide, CRC Handbook of Chemistry and Physics, 83 rd Edition 2002-2003 ed.; CRC Press LLC: New York, 2002). Thus, in some embodiments, the slower reaction rate favors the formation of larger Au DSNPs. Accordingly, in some embodiments, the formed Au DSNPs are covered with a monolayer of dendrimer molecules. Additionally, Au NPs (e.g., with diameter less than 5 nm) can be prepared by fast reduction process using NaBH₄ (See, e.g., Grohn et al., Macromolecules 2000, 33, 6042-6050; Esumi et al., Langniuir 2000, 16, 2604-2608; Kim et al., Chem. Mater. 2004, 16, 167-172). Thus, in some embodiments (e.g., fast reduction using NaBH₄), the dendrimer itself serves as a template to encapsulate Au NPs.

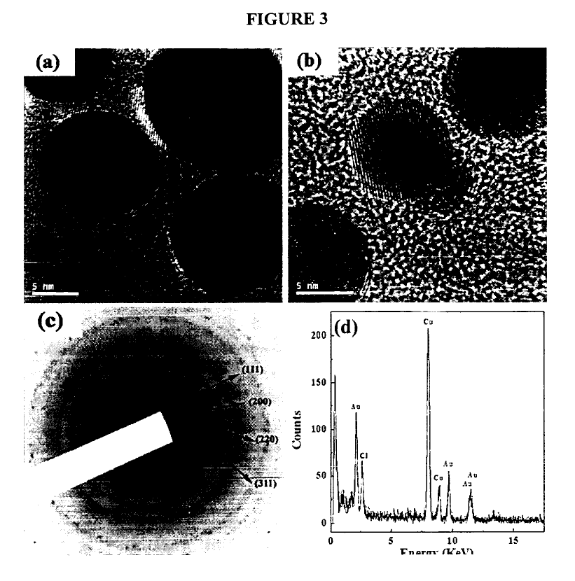

The synthesized Au DSNPs are highly polycrystalline as shown by both high-resolution TEM images (See, e.g., FIG. 3a and FIG. 3b) and SAED patterns (See, e.g. FIG. 3c). In FIG. 3a and FIG. 3b, typical crystal lattices for both single crystals and twin crystals of Au are clearly observed. A typical SAED pattern of {(Au⁰)₉₈-E6.NH₂} DSNPs (FIG. 3c) clearly shows the (111), (200), (220) and (311) rings, indicating the face-centered-cubic (fcc) crystal structures. Thus, the present invention provides for the first time high crystallite of Au DSNPs (e.g., as demonstrated by high resolution TEM). In order to confirm the composition of the formed Au DSNPs, energy dispersive spectroscopy (EDS) was collected for each Au DSNP sample. A typical EDS spectrum of {(Au⁰)₂₄-E4.NH₂} DSNPs (See FIG. 3d) clearly indicated the existence of Au elements.

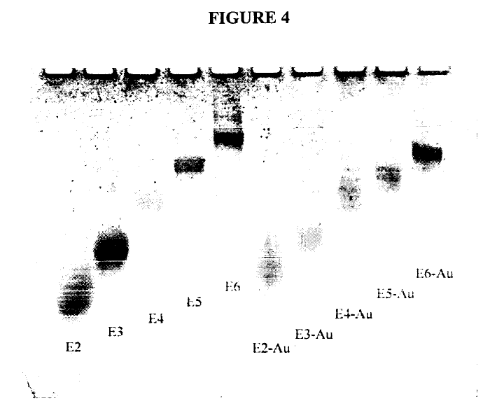

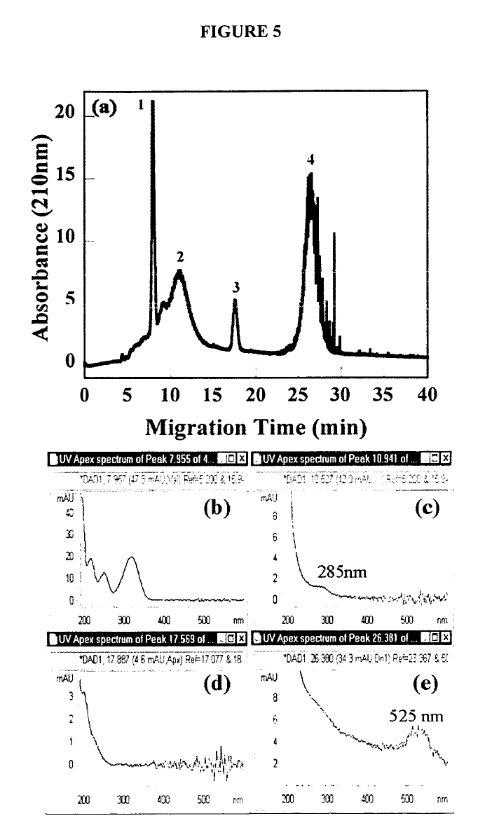

Zeta potential measurements confirmed that the synthesized Au DSNPs are positively charged (See Table 1, above) with zeta potentials ranging from 26.42 to 41.11 mV. Thus, in some embodiments, after the formation of the hybrid nanostructures, the terminal amines of the dendrimers are still available to be protonated. The surface charge polarity of Au DSNPs is similar to the corresponding protonated dendrimers, which was further confirmed by polyacrylamide gel electrophoresis (PAGE) measurements. FIG. 4 shows the PAGE electropherograms of both Au DSNPs and the corresponding dendrimer stabilizers. The Au DSNPs display very similar migration patterns as those of their respective dendrimer stabilizers. In some cases, the difference is that the Au DSNPs exhibit somewhat lower electrophoretic mobility than their corresponding dendrimer stabilizers due to their lower charge/mass ratios after “loading” with Au nanocrystals. PAGE measurements verified the existence of dendrimers for each Au DSNPs, because commercial negatively citric acid-protected Au NPs migrate upward toward the cathode during electrophoresis under the reverse polarity and the comassie-stained band of Au DSNPs are exclusively related to stained dendrimers. The PAGE results also indicate that the formed Au DSNPs are highly stable and both Au nanocrystals and dendrimers do not separate from each other during the electrophoresis at pH 8.3. The existence of Au element was also confirmed in the respective PAGE bands of all Au DSNP samples by collecting the EDS spectra of the respective sliced gel films. Control gel samples without the comassie blue staining and gels bands of respective dendrimer stabilizers stained with comassie blue do not show the presence of Au signals.

Accordingly, in some embodiments, the present invention provides Au DSNPs prepared and stabilized amine-terminated PAMAM dendrimers (e.g., of different generations with the same molar ratios of terminal nitrogen ligands/gold atoms). In some embodiments, the synthesized Au DSNPs display a UV-Vis spectra plasmon peak at 525 nm, while fluorescence spectroscopy demonstrated a strong emission at 458 nm. In some embodiments, the DSNPs are highly polycrystalline with fcc crystal structures. In some embodiments, the size of Au DSNPs decreases with the increase of the number of dendrimer generations. In some embodiments, the formed Au DSNPs are positively charged (e.g., indicating that the protonation state of dendrimer stabilizers is not significantly influenced after the formation of the hybrid nanostructures). In some embodiments, the formed Au DSNPs are stable and integrated (e.g., in preferred embodiments, each Au DSNP is considered as an entire entity). In some preferred embodiments, the amine groups of dendrimers residing on the surface of Au DSNPs can be used for linking functional groups (e.g., biologic ligands) or can be modified with acetyl and hydroxyl groups (See below). Thus, in preferred embodiments, the present invention provides a promising strategy to modify Au DSNPs for biologic sensing, targeting and therapeutics (e.g., cancer therapy).

### Example 2

### Materials and Methods for Post-Synthetic Modification of Dendrimer-Encapsulated Nanoparticles

Materials. Ethylenediamine core amine-terminated PAMAM dendrimers of generation 5 (G5.NH₂, also referred to herein as E5.NH₂, where E denotes the ethylenediamine core of the dendrimer) with a polydispersity index less than 1.08 were purchased from Dendritech (Midland, Mich.). All other chemicals were obtained from Aldrich and used as received. Water used in all of the experiments was purified using a Milli-Q Plus 185 water purification system (Millipore, Bedford, Mass.) with resistivity higher than 18 MΩ.cm. Regenerated cellulose dialysis membranes (MWCO=10,000) were acquired from Fisher.

Synthesis and post modification of dendrimer encapsulated nanoparticles (e.g., Au DENPs). The synthesis of dendrimer-encapsulated nanoparticles (DENPs) can be performed as described (See, e.g., Manna et al., Chew. Mater. 2001, 13, 16741681; Kim et al., Chem. Mater. 2004, 16, 167-172). The Au DENPs were prepared using sodium borohydride reduction chemistry with the dendrimer terminal amine/gold atom molar ratio at 1:0.4. Briefly, 5 mL HAuCl₄ solution (118.2 mM) was added into 20 mL E5.NH₂ aqueous solution (0.577 mM) under vigorous stirring. After 30 min, 6 mL NaBH₄ solution (1.18 mM) dissolved into water/methanol (1:2 in volume) mixture was slowly added to the gold salt/dendrimer mixture while stirring. The reaction mixture turned to a yellow or brown color within a few seconds after addition of the first drop of the NaBH₄ solution. The stirring was continued for 2 h to complete the reaction. The reaction mixture was extensively dialyzed against water (6 times 4 liters) for 3 days to remove the excess of reactants, followed by lyophilization to get pure product {(Au⁰)_{51.2}-E5.NH₂}.

An acetylation reaction procedure was used to modify dendrimer encapsulated nanoparticles (e.g., {(Au⁰)_{51.2}-E5.NH₂}) with acetamide groups (See, e.g., Majoros et al., Macromolecules 2003, 36, 5526-5529). Briefly, 198 μL of triethylamine was added to a 10-mL methanol solution containing 107.86 mg {(Au⁰)_{51.2}-E5.NH₂} DENPs. Methanolic solution (5 mL) of acetic anhydride (144.88 mg, 400% molar excess of the total primary amines of {(Au⁰)_{51.2}-E5.NH₂}) was slowly added (e.g., dropwise) into the DENPs/triethylamine mixture solution while vigorously stirring and the mixture allowed to react for 24 h. The methanolic solution of the reaction mixture was extensively dialyzed against PBS buffer (3 times 4 liters) and water (3 times 4 liters) for 3 days to remove the excess of reactants and byproducts, followed by lyophilization to get pure product (e.g., {(Au⁰)_{51.2}-E5.NHAc}).

A hydroxylation reaction procedure was used to modify dendrimer encapsulated nanoparticles (e.g., {(Au⁰)_{51.2}-E5.NH₂}) with hydroxyl groups (See, e.g., Quintana et al., Pharm. Res. 2002, 19, 1310-1316; Shi et al., Colloids and Surface A: Physicochemical Engineering Aspects 2006, 272, 139-150; Shi et al., Polymer 2005, 46, 3022-3034). To a 10-mL methanol solution containing 108.3 mg of {(Au⁰)_{51.2}-E5.NH₂} DENPs, a methanol solution containing 211.12 mg glycidol (400% molar excess of the amine groups of {(Au⁰)_{51.2}-E5.NH₂}) was added dropwise while stirring. The reaction was stopped after 24 h and the reaction mixture dialyzed against water (6 times 4 liters) for 3 days to remove byproducts and the excess of reactants, followed by lyophilization to get pure product {(Au⁰)_{51.2}-E5.NGlyOH}.

¹H NMR. ¹H NMR spectra of Au DENPs were recorded on a Bruker DRX 500 nuclear magnetic resonance spectrometer. Samples were dissolved in D₂O to give a concentration of approximately 5 mg/mL before NMR measurements.

UV-Vis spectrometry. UV-Vis spectra were collected using a Lambda 20 UV-Vis Spectrometer. All samples were dissolved in water at the concentration of 1 mg/mL.

Zeta potential measurements. Zeta potential measurements were performed using a PSS/NICOMP 380 ZLS particle sizing system (Santa Barbara, Calif.) with a red-diode laser at 635 nm in a multiangle cell.

TEM. A JEOL 2010F analytical electron microscope was performed at 200 kV with an EDS system attached. 5 μL aqueous solution of Au DENPs (0.2 mg/mL) was dropped onto carbon-coated copper grid and air dried before measurements.

PAGE. Analysis of PAMAM dendrimers and Au DENPs by PAGE was performed on a Micrograd vertical electrophoresis system (Model FB-VE10-1, FisherBiotech, Pittsburgh, Pa.) with a commercial power supply (Model EC135-90; Thermo Electron Corporation, Milford, Mass.). Precast 4-20% gradient express gels for PAGE were obtained from ISC BioExpress (Kaysville, Utah). Tris-Glycine (TG) native buffer (pH=8.3) was purchased from Invitrogen (Carlsbad, Calif.). It was diluted by a factor of ten to prepare the running buffer. PAGE separations typically required 50 min at 200 V. Reverse polarity was used for the analysis of the polycationic PAMAM dendrimers and Au DENPs. Into each sample well 2 μL of a sample solution composed of equal volume of 1 mg/mL PAMAM dendrimers (amine, acetamide, hydroxyl-terminated E5 dendrimers) or Au DENPs and methylene blue sucrose dye solutions (50% sucrose, 1% methylene blue) was injected. Developed gels were stained with 0.025% Comassie Blue R-250 in 40% methanol and 7% acetic acid aqueous solution overnight. The gels were destained with 7% (v/v) acetic acid and 5% (v/v) methanol in water.

### Example 3

### Analysis of Post-Synthetic Modification of Dendrimer-Encapsulated Nanoparticles

The present invention provides a new, facile approach to generate functionalized dendrimer-encapsulated nanoparticles (e.g., Au DENPs) with different functional groups (e.g., through the modification of the terminal amine groups of PAMAM templates, See Example 2, above).

Preformed Au DENPs using amine-terminated PAMAM dendrimers of generation 5 (E5.NH₂) as templates were reacted with acetic anhydride or glycidol molecules to form acetamide or hydroxyl-functionalized Au DENPs (See Example 2 and FIG. 6). Thus, in some embodiments, the present invention provides a novel method of modifying Au DENP surfaces through dendrimer-mediated conventional organic reactions. The formed Au DENPs after surface functionalization are stable, water-soluble, and display similar size distributions and optical properties, while their surface charge polarity changes. Accordingly, in some embodiments, the present invention provides the ability to directly tailor the surface functionalities of preformed Au DENPs.

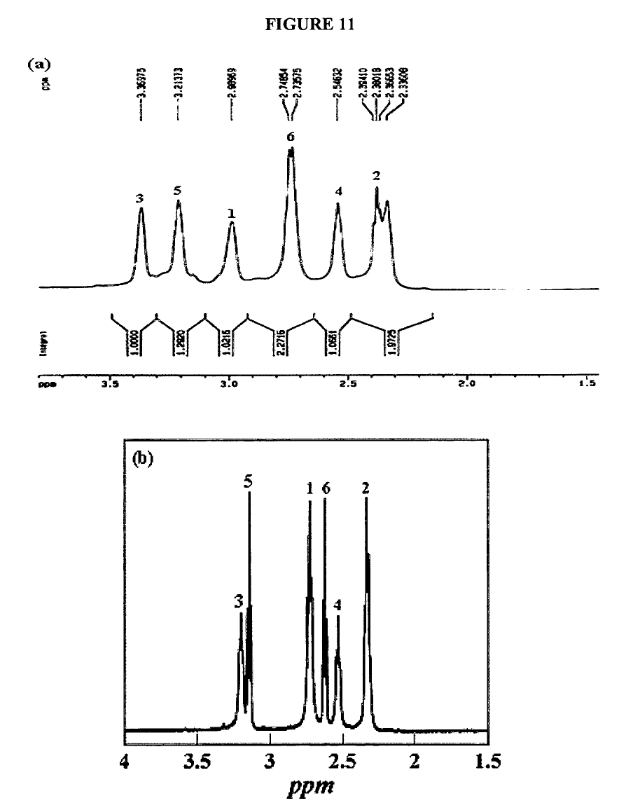

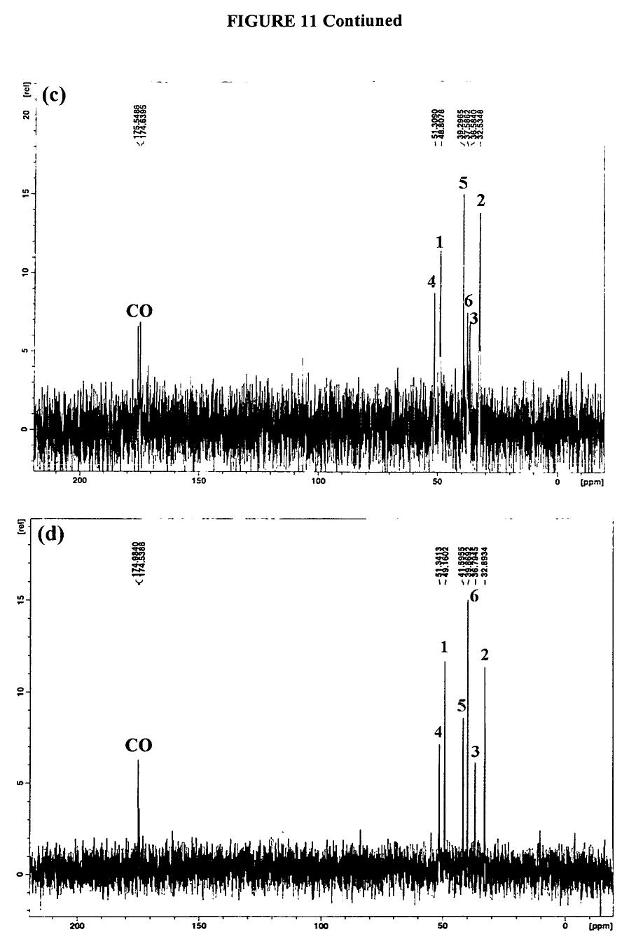

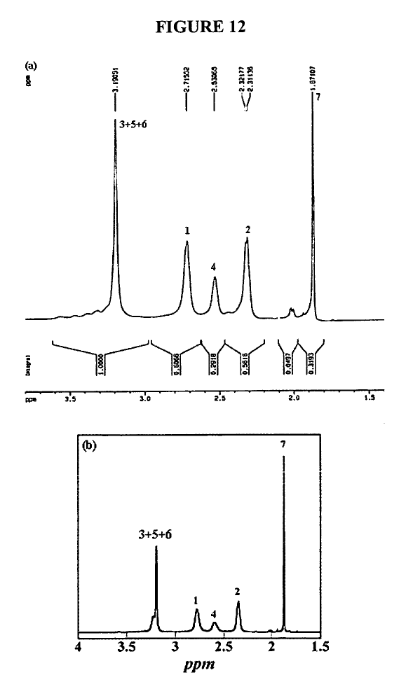

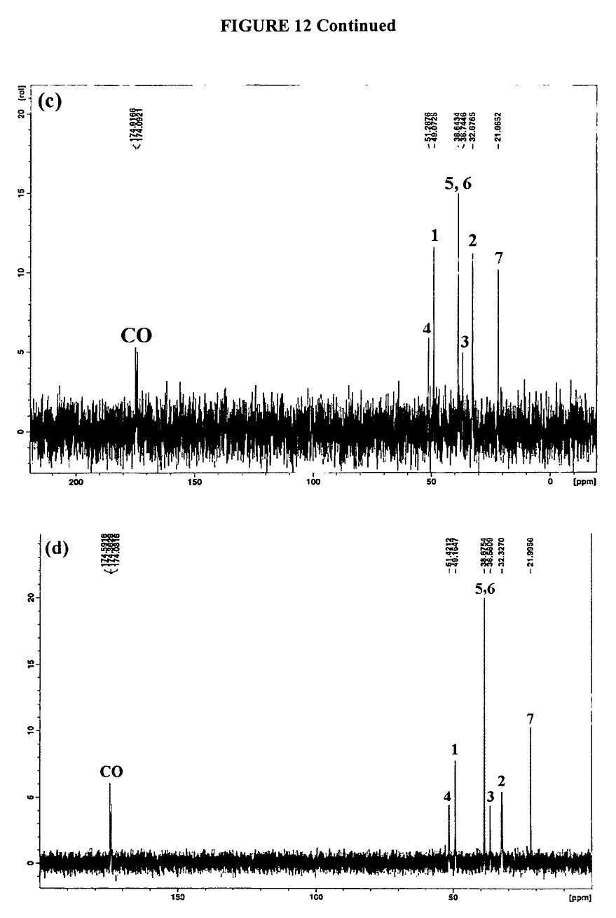

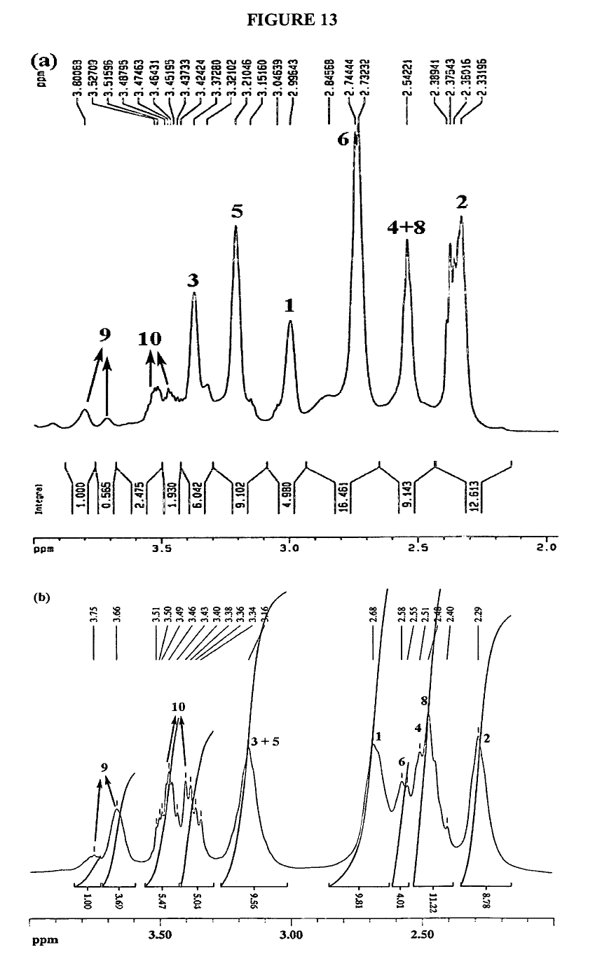

The surface modifications of the formed {(Au⁰)_{51.2}-E5.NH₂} DENPs with acetamides and hydroxyl groups were confirmed by ¹H NMR measurements. ¹H NMR and ¹³C NMR spectra of Au DENPs with amine, acetamide, and hydroxyl groups and the corresponding dendrimers G5.NH₂, G5.NHAc, and G5.NGlyOH are shown in FIGS. 11, 12, and 13, respectively. ¹H NMR spectra of Au DENPs with amine, acetamide, and hydroxyl groups are described in Example 2. The ¹H NMR spectra of {(Au⁰)_{51.2}-E5.NH₂}, {(Au⁰)_{51.2}-E5.NHAc}, and {(Au⁰)_{51.2}-E5.NGlyOH} DENPs are very similar to those of E5.NH₂, E5.NHAc, and E5.NglyOH. dendrimers (See, e.g., Shi et al., Polymer 2005, 46, 3022-3034). Although an understanding of the mechanism is not necessary to practice the present invention and the present invention is not limited to any particular mechanism of action, the slight down-field shift of —CH₂— proton signals related to {(Au⁰)_{51.2}-E5.NH₂} (from 3.20, 3.14, 2.73, 2.62, 2.53, 2.33 ppm of E5.NH₂ to 3.37, 3.21, 2.99, 2.75, 2.55, 2.38 ppm, respectively), in some embodiments, are likely due to the strong interaction between some of the dendrimer terminal amine groups and the Au NPs. Similarly, although an understanding of the mechanism is not necessary to practice the present invention and the present invention is not limited to any particular mechanism of action, the slight variation of the NMR spectrum of {(Au⁰)_{51.2}-E5.NGlyOH} from that of E5.NGlyOH may be due, in some embodiments, to much more incomplete hydroxylation reaction (e.g., because some of the dendrimer terminal amines bound with Au NPs are not available to react with glycidol molecules). The incomplete reaction is also verified by polyacrylamide gel electrophoresis (PAGE) (see below). However, the ¹H NMR spectra of both {(Au⁰)_{51.2}-E5.NHAc} and E5.NHAc are similar because the acetylation reaction is very fast and the binding of dendrimer terminal amines with Au NPs does not affect the reaction.

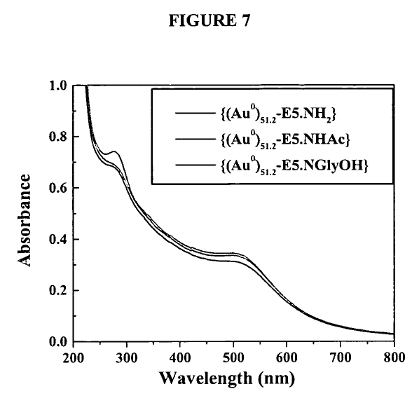

The optical properties of the formed and modified Au DENPs were investigated using UV-Vis spectrometry. UV-Vis spectra of {(Au⁰)_{51.2}-E5.NH₂}, {(Au⁰)_{51.2}-E5.NHAc} and {(Au⁰)_{51.2}-E5.NGlyOH} DENPs show that they exhibit similar absorption behaviors with surface plasmon peaks around 510 nm, indicating their similar sizes and size distributions (See FIG. 7).

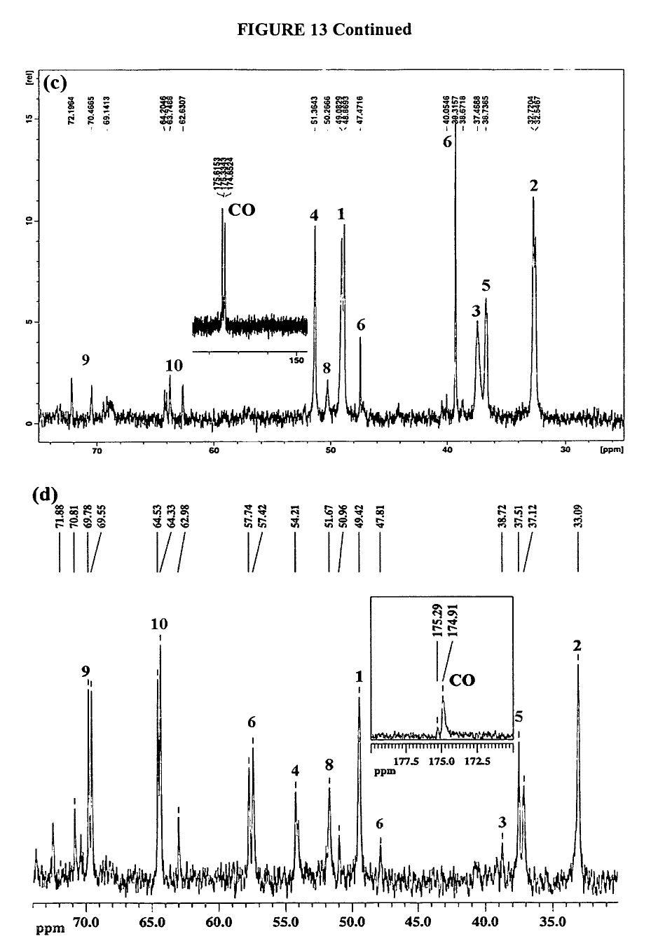

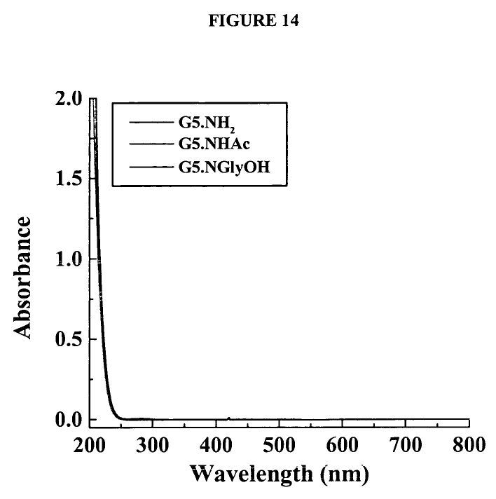

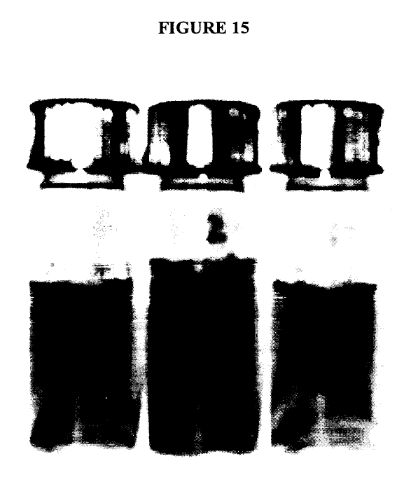

The corresponding surface modified dendrimers in the absence of the Au NPs do not show any absorption features at wavelengths above 250 nm (UV-vis spectra of G5.NH₂, E5.NHAc, and E5.NGlyOH dendrimers are shown in FIG. 14). The Au DENPs are soluble and stable in water, and no aggregates formed for at least 10 months after synthetic modifications with either acetic anhydride or glycidol molecules (a photograph of the aqueous solutions of the Au DENPs is shown in FIG. 15)

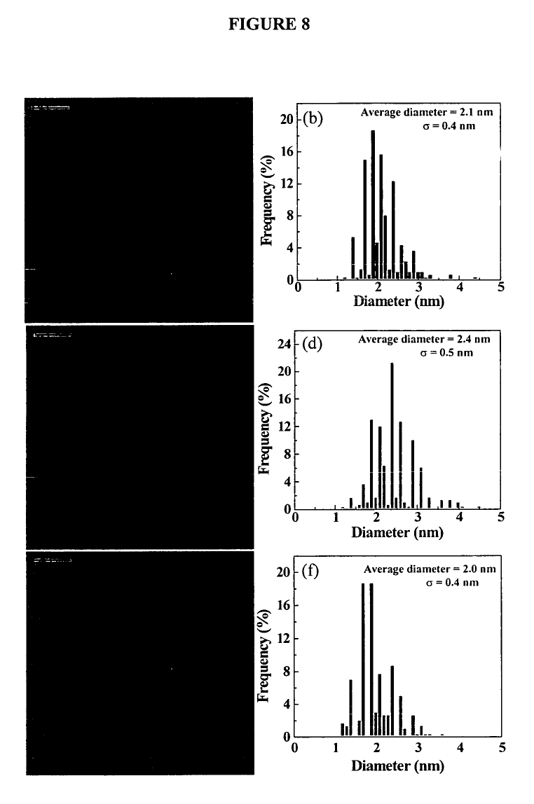

The morphology and size distribution of synthesized Au DENPs were observed by transmission electron microscopy (TEM) imaging (See FIG. 8). The Au DENPs appear to be monodispersed and small with sizes ranging from 2.0±0.4 to 2.4±0.5 nm. High resolution TEM images (insets of FIGS. 2a, 2c, and 2e) show that all Au DENPs are crystalline. The crystalline nature of the Au DENPs was also confirmed using selected area electron diffraction (SAED). The (111), (200), (220) and (311) rings in the SAED patterns indicate face-centered-cubic (fcc) crystal structures. It has been shown that dendrimer-stabilized Au NPs (size around 7-15 nm) formed using hydrazine reduction chemistry display high crystallites (See Example 1, above). Thus, in some embodiments, the high crystallite nature of Au NPs is not influenced upon dendrimer stabilization or encapsulation (e.g., in the latter case, even if they are small in sizes). Energy dispersive spectroscopy (EDS) of each Au DENP samples indicates the existence of Au elements.

Although the optical properties and size distributions of the Au DENPs are similar, their surface charges change. Zeta potential measurements show that the surface charge potentials of Au DENPs follow the order of {(Au⁰)_{51.2}-E5.NH₂} (+36.86 mV)>{(Au⁰)_{51.2}-E5.NGlyOH} (+23.47 mV)>{(Au⁰)_{51.2}-E5.NHAc} (+4.27 mV). The zeta potential changes indicate the successful surface modification of {(Au⁰)_{51.2}-E5.NH₂} DENPs. Thus, in some embodiments, the present invention provides a method of manipulating the surface charge potentials of Au DENPs through dendrimer-mediated conventional organic synthesis.

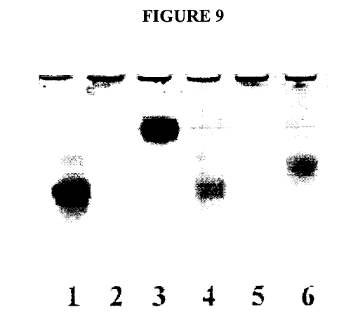

The water solubility and stability of Au DENPs do not change after post synthetic modifications with acetamides and hydroxyl groups. PAGE analysis of the synthesized and modified Au DENPs and the corresponding dendrimer derivatives (See FIG. 9) show that Au DENPs exhibit similar migration patterns to those of the corresponding dendrimer derivatives. {(Au⁰)_{51.2}-E5.NGlyOH} migrates faster than E5.NGlyOH dendrimer, which may be attributed to the much more incomplete hydroxylation reaction after loading with Au NPs. This is consistent with the ¹H NMR results. The slight broader bands of Au DENPs resulted from increased polydispersity of Au DENPs compared with corresponding dendrimers without loading of Au nanocrystals.

Thus, the present invention provides small monodispersed Au DENPs with different surface functionalities (e.g., acetamide or hydroxyl groups). The Au DENPs are crystalline and have sizes in the range of 2.0-2.4 nm. The functionalization of Au DENPs does not influence their sizes, size distributions, crystallites, water solubility, and stability. The surface charge potentials of Au DENPs are varied depending on their surface functional groups (e.g., acetamide versus hydroxyl). Accordingly, the present invention provides compositions comprising post-synthetic modification of Au DENPs, and methods of using these compositions to generate dendrimer encapsulated nanoparticles (e.g., Au DENPs) with surface functional groups (e.g., therapeutic agents, targeting agents, imaging agents, biological monitoring agents). For example, in some embodiments, a composition comprising post synthetically modified Au DENPs (e.g., with a hydroxyl or acetamide group) can be used to generate (e.g., through attachment of one or more functional groups to the hydroxyl or acetamide reactive sites) Au DENPs conjugated to one or more functional groups (e.g., a therapeutic agent, a targeting agent, an imaging agent, and/or a biological monitoring agent).

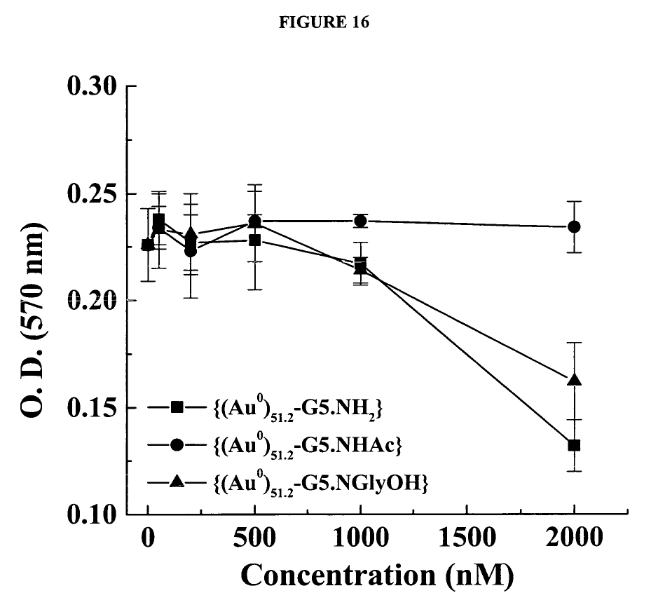

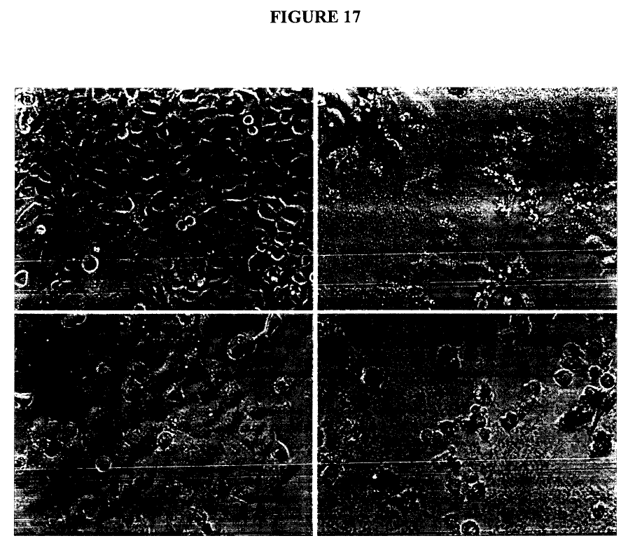

The cytotoxicity of the synthesized Au DENPs was evaluated by an MTT (3-(4,5-dimethylthiazol-2-yl)-2,5-diphenyltetrazolium bromide) assay of KB cells (a human epithelial carcinoma cell line) (See FIG. 16). All Au DENPs are non-toxic below the concentration of 1.0 μM. Above 1.0 μM, the cytotoxicity of Au DENPs follows the order of {(Au⁰)_{51.2}-E5.NH₂}>{(Au⁰)_{51.2}-E5.NGlyOH}>{(Au⁰)_{51.2}-E5.NHAc}, related to the degree of cationic surface charge. The toxicity of Au DENPs was also evaluated by visualizing the morphologies of KB cells treated with different surface-functionalized Au DENPs. At the concentration of 2.0 μM, the morphology of KB cells treated with {(Au⁰)_{51.2}-E5.NHAc} DENPs is similar to the morphology of untreated KB cells, providing that {(Au⁰)_{51.2}-E5.NHAc} DENPs display a very good biocompatibility (See FIG. 17). Acetylation of {(Au⁰)_{51.2}-E5.NH₂} DENPs neutralizes the surface charges of Au NPs, as confirmed by PAGE and zeta-potential measurements, making them highly compatible with biological systems. In contrast to the acetylation reaction, much less complete hydroxylation of {(Au⁰)_{51.2}-E5.NH₂} DENPs (as compared with hydroxylation of E5.NH₂ dendrimers) cannot effectively neutralize their positive charges; therefore, the formed {(Au⁰)_{51.2}-E5.NGlyOH} DENPs still display some cytotoxicity at high concentrations. The toxicity data of {(Au⁰)_{51.2}-E5-NH₂} and {(Au⁰)_{51.2}-E5.NHAc} DENPs are comparable with the corresponding dendrimer derivatives in the absence of Au NPs (See FIG. 18).

However, E5.NGlyOH dendrimers do not exhibit toxicity even at a concentration of 2.0 μM, providing that hydroxylation of E5.NH₂ dendrimers to form E5.NGlyOH significantly decreases the surface charge of the dendrimers. Thus, the present invention provides that post-synthetic modification of Au DENPs is a straightforward approach to designing non-toxic Au NPs for biological applications.

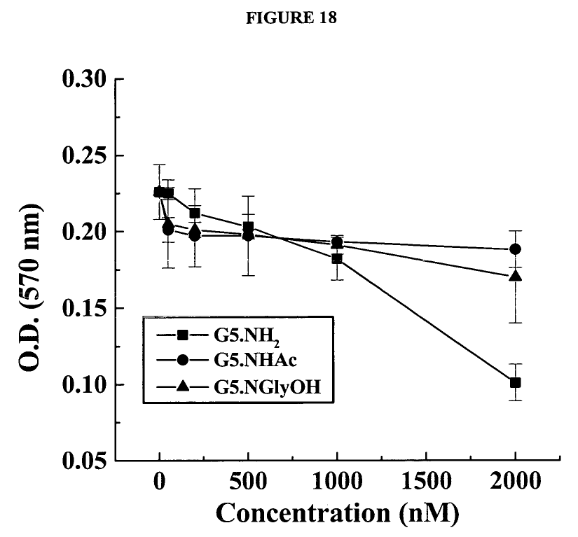

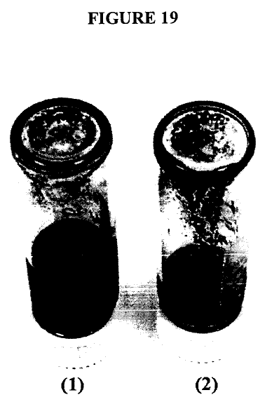

Attempts were made to synthesize non-toxic Au NPs using preformed E5.NHAc and E5.NGlyOH dendrimers as templates under similar conditions. In both cases, black precipitates were fonrmcd (A photograph of the aqueous solutions of Au NPs synthesized using E5.NHAc and E5.NGlyOH dendrimers as templates is shown FIG. 19). Thus, the present invention provides that the complexation of AuCl₄⁻ ions with either the acetamide or glycidol hydroxyl-terminated E5 dendrimer is much weaker than that with amine-terminated E5 dendrimers, significantly decreasing the stability of the Au NPs.

### Example 4

### Cancer Cell Targeting and Imaging Using Dendrimer-Encapsulated Nanoparticles

### Materials and Methods.

Synthesis and functionalization of Au DENPs. The procedure to synthesize Au DENPs was similar to that described in Examples 1 and 2. The Au DENPs were prepared using sodium borohydride reduction chemistry with the dendrimer terminal amine/gold atom molar ratio at 1:0.4. Briefly, 5 mL HAuCl₄ solution (118.2 mM) was added into 20 mL G5.NH₂ (G denotes generation) dendrimer (Dendritech, Midland, Mich.) aqueous solution (0.577 mM) under vigorous stirring. After 30 min, 6 mL NaBH₄ solution (197 mM) dissolved into water/methanol (2:1 in volume) mixture was slowly added to the gold salt/dendrimer mixture while stirring. The reaction mixture turned to a deep red color within a few seconds after addition of the NaBH₄ solution. The stirring was continued for 2 hours to complete the reaction. The reaction mixture was extensively dialyzed against water (6 times 4 liters) for 3 days to remove the excess of reactants, followed by lyophilization to get product {(Au⁰)_{51.2}-G5.NH₂} DENPs.

Five molar equivalents of fluorescein isothiocyanate (FI) (3.27 mg, 8.4 μmol) (Aldrich) dissolved in DMSO (5 mL) were dropwise added to a solution of {(Au⁰)_{51.2}-G5.NH₂} DENPs (61.19 mg, 1.68 μmol) in DMSO (10 mL) in a nitrogen atmosphere under vigorous magnetic stirring. After 24 h, the reaction mixture (15 mL) was divided into 2 aliquots with equal volume (7.5 mL). For Aliquot 1, the FI-labeled {(Au⁰)_{51.2}-G5.NH₂} DENPs were acetylated to convert the remaining amino groups of G5 dendrimer to acetamide groups according to a described methods (See, e.g., Majoros et al., Macromolecules 2003, 36, 5526. Briefly, Aliquot 1 was added with triethylamine (37.4 mg, 0.37 mmol) and mixed well, followed by dropwise addition of an acetic anhydride solution (37.74 mg, 0.37 mmol) under vigorous stirring. After 24 h, the reaction mixture was extensively dialyzed against PBS buffer (3 times 4 liters) and water (3 times 4 liters) using regenerated cellulose dialysis membranes (MWCO=10,000) (Fisher) for 3 days to remove the excess of reactants and byproducts, followed by lyophilization to get product {(Au⁰)_{51.2}-G5-FI₅-Ac} DENPs (yield=59.3%). ¹H NMR (500 MHz, D₂O) δ 6.99 (bs, 2H), 6.39 (bs, 4H), 3.10 (s, 174H), 2.67 (s, 107H), 2.43 (s, 73H), 2.25 (s, 118H), 1.78 (s, 102H). The numbers of dendrimer protons were extrapolated based on the integration values relative to those for FI protons.

For Aliquot 2, the FI-labeled {(Au⁰)_{51.2}-G5.NH₂} DENPs were further modified with folic acid (FA) (Aldrich) according to a described procedure (See, e.g., Choi et al., Chem. Biol. 2005, 12, 35) with a slight modification. Briefly, a 5-molar equivalent of folic acid (FA) (1.90 mg, 4.3 μmol) (Aldrich) in 2 mL DMSO was mixed with a 2-mL DMSO solution containing EDC (34.8 gmol, 6.67 mg) (Aldrich) and stirred for 3 h. This process activated the γ-carboxylic acid of FA for further reaction with FI-labeled {(Au⁰)_{51.2}-G5.NH₂} DENPs. The activated FA solution (4 mL in DMSO) was added to Aliquot 2-FI-labeled {(Au⁰)_{51.2}-G5.NH₂} DENPs and stirred for 3 days. Then, the FI- and FA-modified {(Au⁰)_{51.2}-G5.NH₂} DENPs were further acetylated to neutralize the remaining amino groups of G5 dendrimers, followed by extensive dialysis against PBS buffer (3 times 4 liters) and water (3 times 4 liters) for 3 days to remove the excess of reactants and byproducts and lyophilization to get product {(Au⁰)_{51.2}-G5-FI₅-FA₅-Ac} DENPs (yield=77.1%). ¹H NMR (500 MHz, D₂O) δ 8.60 (bs, 1H), 7.46 (bs, 2H), 6.93 (bs, 2H), 6.50 (bs, 2H), 6.37 (bs, 4H), 3.09 (s, 174H), 2.66 (s, 107H), 2.48 (s, 73H), 2.23 (s, 118H), 1.78 (s, 102H).

Instrumentation for Characterization of Functionalized Au DENPs. ¹H NMR. ¹H NMR spectra of Au DENPs were recorded on a Bruker DRX 500 nuclear magnetic resonance spectrometer. Samples were dissolved in D₂O before NMR measurements.

UV-Vis spectrometry. UV-Vis spectra were collected using a Perkin Elmer Lambda 20 UV-Vis Spectrometer. All samples were dissolved in water at the concentration of 1 mg/mL.

Zeta potential measurements. Zeta potential measurements were performed using a Malvern Zetasizer Nano ZS model ZEN3600 (Worcestershire, UK) equipped with a standard 633 nm laser.

Transmission electron microscopy (TEM). A JEOL 2010F analytical electron microscope was performed at 200 kV with an EDS system attached. A 5-μL aqueous solution of Au DENPs (1 mg/mL) was dropped onto a carbon-coated copper grid and air dried before measurements.

Cell Cultures and Biological Evaluation. The KB cells (ATCC, CLL17, Rockville, Md.) were continuously grown in two 24-well plates, one in rA-free media and the other in regular RPMI 1640 medium (Gibco/BRL, Gaithersburg, Md.) supplemented with penicillin (100 units/mL) (Sigma, St. Louis, Mo.), streptomycin (100 μg/mL) (Sigma, St. Louis, Mo.), 10% heat-inactivated FBS, and 2.5 μM FA. The cells grown in FA-free media express high-level folic acid receptor (FAR), while the cells grown in FA-containing media express low-level FAR.

MTT cytotoxicity assay. An MTT (3-(4,5-dimethylthiazol-2-yl)-2,5-diphenyltetrazolium bromide) assay was used to quantify the viability of cells. Briefly, 5×10⁴ KB cells per well were seeded into a 96-well plate. After overnight incubation, functionalized Au DENPs at concentrations ranging from 0 to 2 μM in PBS (pH 7.4) was added. After 24 h incubation with Au DENPs at 37° C., MTT reagent in PBS solution was added. The assays were carried out according to manufacturer's instructions. For each concentration of Au DENPs, 5 wells of cells were analyzed.

Flow cytometry analysis. Approximately 2×10⁶ cells per well were seeded in 12-well plates the day before the experiments. An hour before initiating an experiment, the cells were rinsed four times with serum-free and FA-deficient RPMI 1640 media. Functionalized Au DENPs were added in the final concentrations of 0-300 nM. After 1 h incubation with the functionalized Au DENPs, KB cells with both high and low-level FAR expression were trypsinized (Gibco/BRL, Gaithersburg, Martyland) and suspended in PBS containing 0.1% bovine serum albumin (Sigma, St. Louis, Mo.) and analyzed using a Becton Dickinson FACScan analyzer. The FL1-fluorescence of 10 000 cells was measured, and the mean fluorescence of gated viable cells was quantified.

Confocal microscopy. Confocal microscopic analysis was performed in cells plated on a plastic cover-slip using an Olympus FluoView 500 laser scanning confocal microscope (Melville, N.Y.). FI fluorescence was excited with a 488-nm argon blue laser and emission was measured through a 505-525 barrier filter. The optical section thickness was set at 5 μm. The cells were incubated with functionalized Au DENPs for 2 h, followed by rinsing with PBS buffer. The nuclei were counterstained with 1 μg/mL of Hoescht33342, using a standard procedure. Samples were scanned using a 60× water immersion objective lens and magnified with FluoView software.

Transmission electron microscopy (TEM). The uptake of functionalized Au DENPs was further examined by a Phillips CM 100 TEM microscope operating at a voltage of 60 kV. Images were recorded using a Hamamatsu digital camera controlled by AMT (advance microscopy technology) software. The specimens were prepared according to the following procedures. The KB cells with high-level FAR were aliquoted in 5-mL tubes at a concentration of 1×10⁶ cells/mL. After overnight growth at 37° C., the medium was removed and 2% FBS solution containing 50 nM of functionalized Au DENPs was added; the incubation was carried out for 2 h at 37° C. Then, the medium was removed and cells were washed with Sorenson buffer and fixed at room temperature for 1 h using 2.5% of glutaraldehyde in Sorenson buffer. Cells were rinsed 3 times with Sorenson buffer, resuspended in the same medium and post-fixed using 1.0% osmium tetroxide for 1 h. After additional washing in buffer, cells were dehydrated in a series of ethanol solutions of 30%, 50%, 70%, 95%, and 100%. Samples were further infiltrated using the following sequence of mixtures of 100% ethanol and Epon: 3 parts of ethanol+1 part resin (for 1 h), 1 part of ethanol+1 part resin (for 1 h), 1 part of ethanol+3 parts resin (overnight), full-strength resin (4 h), and full-strength resin (overnight). After the third change of resin, polymerization was performed and sections with the thickness of 75 nm were obtained using a Reichart Ultramicrotom. Sections were mounted on 200 mesh copper grids before TEM measurements.

Dendrimer-encapsulated gold nanoparticles (Au DENPS) covalently linked with targeting and imaging molecules.

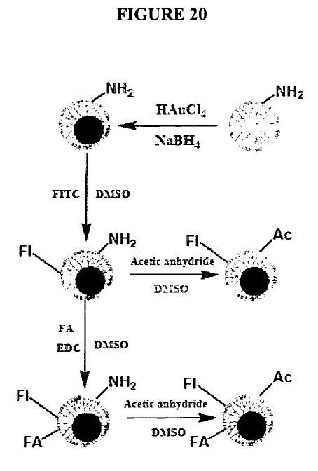

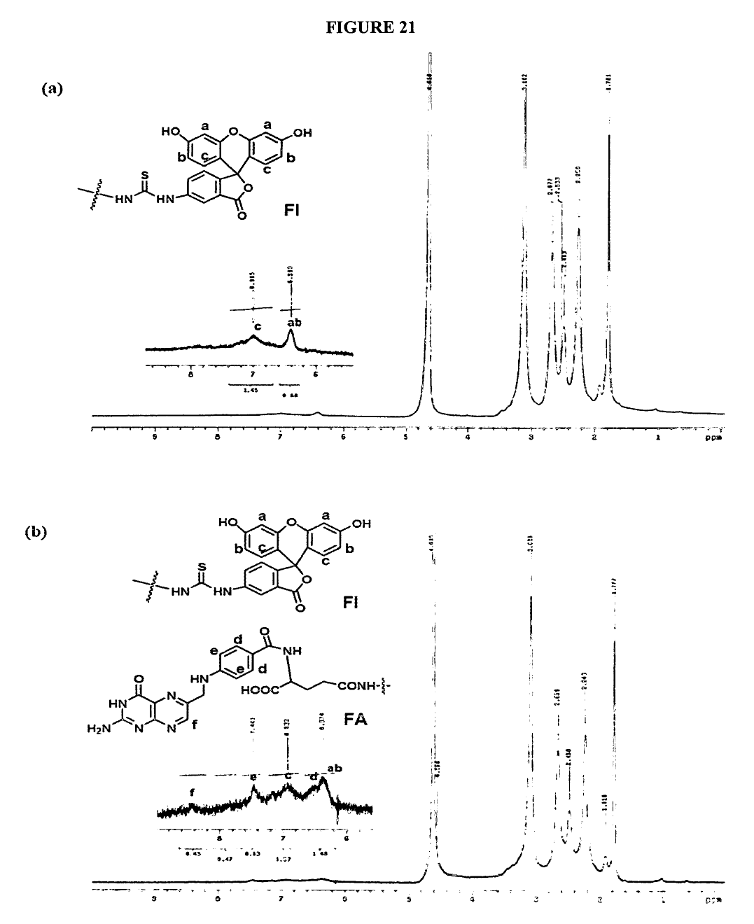

The approach to functionalize the Au DENPs with defined numbers of targeting molecules (e.g. FA and dyes (e.g., FI) (See FIG. 20) were modified from the methods used to functionalize dendrimers (e.g., without encapsulated metal nanoparticles) for targeting and imaging of cancer cells (See, e.g., Majoros et al., Med. Chem. 2005, 48, 5892; J. R. Baker, Jr. et al., Biomed. Microdevices 2001, 3, 61; Quintana et al., Pharm. Res. 2002, 19, 1310). One of the steps in the preparation of FA- and FI-modified Au DENPs was to keep the surface charges on the particles neutral in order to avoid toxicity and non-specific binding. Although not limited to any particular method, this was accomplished by a final acetylation step to convert the remaining amine groups of G5.NH₂ dendrimers to acetamides (See FIG. 20). Zeta potential measurements showed that after the final acetylation step, the surface potentials of the formed {(Au⁰)_{51.2}-G5-FI₅-Ac} (ξ=−1.11 mV) and {(Au⁰)_{51.2}-G5-FI₅-FA₅-Ac} (ξ=−2.30 mV) DENPs (Ac denotes acetyl) are close to neutral, indicating the success of the acetylation reaction. Although an understanding of the mechanism is not necessary to practice the present invention and the present invention is not limited to any particular mechanism of action, in some embodiments, the slight negative charges of both DENPs may be derived from the deprotonated carboxyl groups in both FI and FA moieties being conjugated. The numbers of FI and FA moieties conjugated onto each Au DENP can be estimated by comparing the differences between the integration values of ¹H NMR signals associated with dendrimers and the FI and FA moieties. This was carried out for dendrimer (without encapsulated Au nanoparticles) conjugation (See FIG. 21). The average numbers of FI and FA moieties conjugated onto each Au DENP were estimated to be 4.0 and 4.5, respectively.

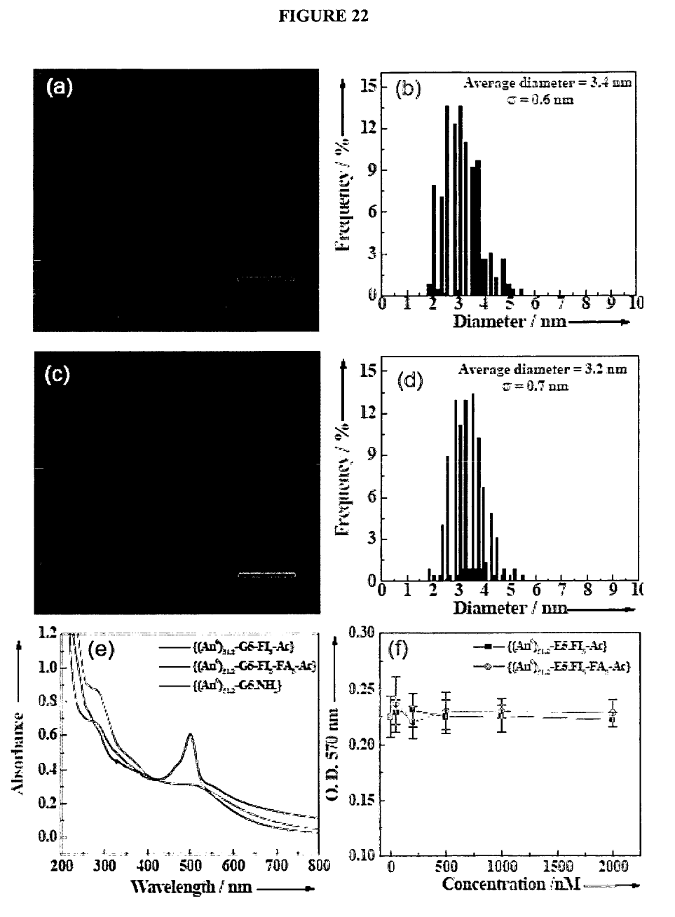

TEM images showed that the sizes of {(Au⁰)_{51.2}-G5-FI₅-Ac} and {(Au⁰)_{51.2}-G5-FI₅-FA₅-Ac} DENPs were 3.4±0.6, and 3.2±0.7 nm (FIG. 2a-d), respectively. Although an understanding of the mechanism is not necessary to practice the present invention and the present invention is not limited to any particular mechanism of action, in some embodiments, the somewhat larger size compared with the pristine {(Au⁰)_{51.2}-G5.NH₂} DENPs (2.1 nm) may be due to multiple surface modifications, that facilitate Ostwald ripening of the Au DENPs. UV-vis spectrometry (FIG. 22e) verified the conjugation of FI and FA moieties onto Au DENPs. The {(Au⁰)_{51.2}-G5-FI₅-FA₅-Ac} DENs showed characteristic absorption peaks at both 500 runm and 280 nm for respective FI and FA moieties, while only the characteristic absorption peak at 500 nm related to FI moiety was observed with {(Au⁰)_{51.2}-G5-FI₅-Ac} DENPs. In addition, a band representing an overlap of the surface plasmon resonance of Au DENPs (510 mm) with the absorption of FI moiety was also observed. The functionalized Au DENPs are stable, and no precipitation of the solution appeared even after periods of storage as long as 9 months. A photograph of the aqueous solutions of {(Au⁰)_{51.2}-G5-FI₅-Ac} and {(Au⁰)_{51.2}-G5-FI₅—FA₅-Ac} DENPs is shown in FIG. 23. An MTT (3-(4,5-dimethylthiazol-2-yl)-2,5-diphenyltetrazolium bromide) assay of KB cells (a human epithelial carcinoma cell line) showed that the functionalized Au DENPs were not cytotoxic even at a concentration up to 2000 nM (FIG. 22f), providing that the final acetylation step creates biocompatible nanoparticles.

Cancer cell binding and internalization using DENPs comprising a folic acid targeting molecule.

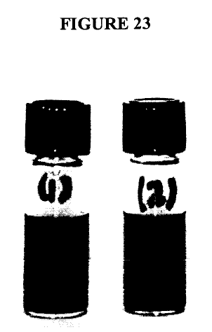

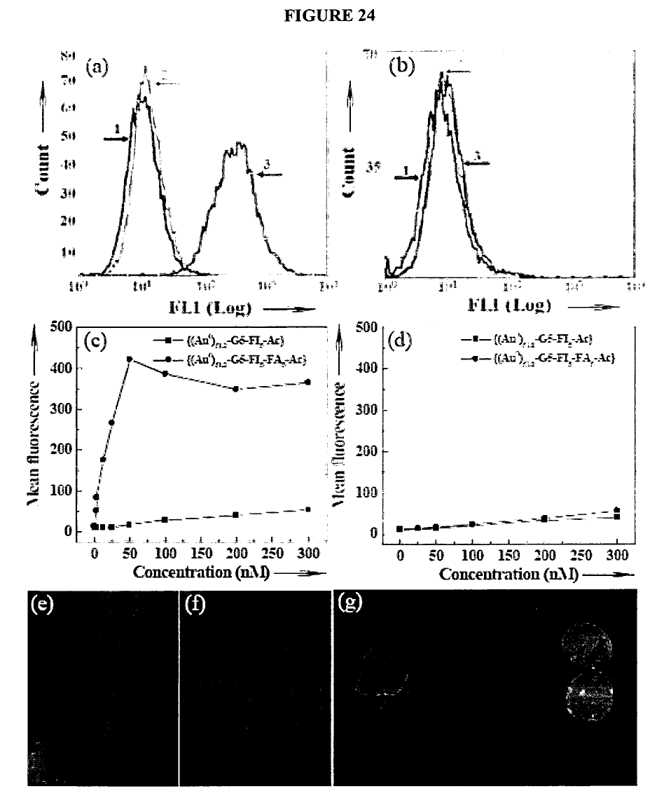

FA has been extensively investigated for targeting various cancer cells, including ovary, kidney, uterus, testis, brain, colon, lung, and myelocytic blood that overexpress FA receptors (FAR) (See, e.g., Campbell, T. A. Jones, W. D. Foulkes, J. Trowsdale, Cancer Res. 1991, 51, 5329; Garin-Chesa, I. Campbell, P. E. Saigo, J. L. Lewis, Jr., L. J. Old, W. J. Rettig, Am. J. Pathol. 1993, 142, 557; Weitman, R. H. Lark, L. R. Coney, D. W. Fort, V. Frasca, V. R. Surawski, B. A. Kamen, Cancer Res. 1992, 52, 3396; Ross, P. K. Chaudhauri, M. Ratnam, Cancer Res. 1994, 73, 2432). The high-affinity FAR for FA (K_{d}=0.1-1 nM) affords specific binding and internalization of FA-modified materials to cancer cells in the presence of normal cells through receptor-mediated endocytosis. KB cells were selected for the specific binding with functionalized Au DENPs. KB cells with both high- and low-levels of FAR were respectively incubated with {(Au⁰)_{51.2}-G5-FI₅-FA₅-Ac} and {(Au⁰)_{51.2}-G5-FI₅-Ac} DENPs for 1 h. FIG. 24a-d shows the flow cytometric analyses of KB cells that express both high- and low-level FAR after exposure to functionalized Au DENPs (25 nM) for 1 h. It is clear that treatment of KB cells expressing high-level FAR with {(Au⁰)_{51.2}-G5-FI₅-FA₅-Ac} DENPs results in a significant increase in the fluorescence signal within the cells. In contrast, the same KB cells treated with {(Au⁰)_{51.2}-G5-FI₅-Ac} DENPs without FA display a similar fluorescence signal to cells treated with PBS buffer (See FIG. 24a), suggesting no binding and/or targeting of the {(Au⁰)_{51.2}-G5-FI₅-Ac} DENPs. KB cells with low-level FAR treated with either {(Au⁰)_{51.2}-G5-FI₅—FA₅-Ac} or {(Au⁰)_{51.2}-G5-FI₅-Ac} DENPs show a similar fluorescence intensity to the PBS control (See FIG. 24b). These results indicate that the specificity of {(Au⁰)_{51.2}-G5-FI₅-FA₅-Ac} DENPs binding to KB cells is restricted to cells containing high levels of FAR. The cellular uptake of the FA-functionalized Au DENPs showed a dose-dependent fashion, with saturation and 50% binding occurring at approximately 50 nM and 18 nM, respectively (See FIG. 24c), which is comparable with the binding capacity of FA-modified G5 dendrimers (See, e.g., Thomas et al., J. Med. Chem. 2005, 48, 3729). For KB cells with low-level FAR, neither {(Au⁰)_{51.2}-G5-FI₅-FA₅-Ac} nor {(Au⁰)_{51.2}-G5-FI₅-Ac} DENPs displayed any significant binding, even at concentrations up to 300 nM (See FIG. 24d).

The conjugation of FI moiety onto Au DENPs also affords confocal microscopic imaging of the intracellular uptake. FIGS. 24e, 24f, and 24g show that only KB cells with high-level FAR treated with FA-modified {(Au⁰)_{51.2}-G5-FI₅-FA₅-Ac} DENPs display fluorescence signals, associated with the specific internalization of {(Au⁰)_{51.2}-G5-FI₅-FA₅-Ac} DENPs into the cytoplasm of the cells (FIG. 24g). In contrast, the same KB cells treated with {(Au⁰)_{51.2}-G5-FI₅-Ac} DENPs without FA modification do not show any fluorescence signals (See FIG. 24f), similar to KB cells treated with PBS buffer (See FIG. 24e). The present invention provides that binding and/or targeting and intracellular uptake do not occur in cells exposed to non-FA modified Au DENPs.

### Subcellular Localization of Internalized DENPs.

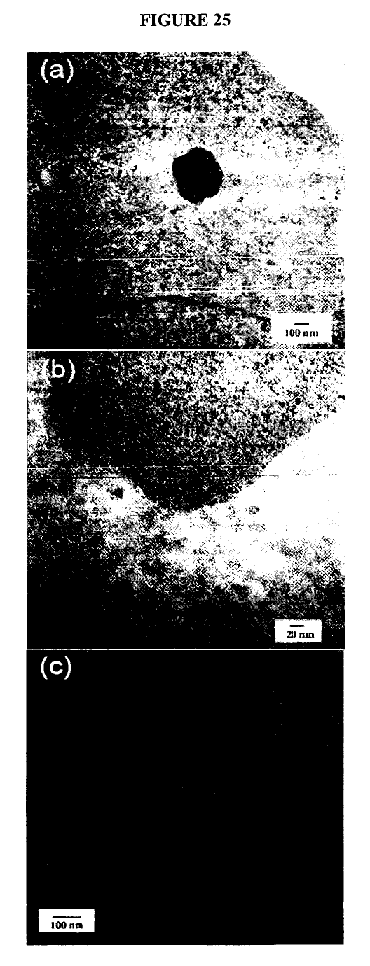

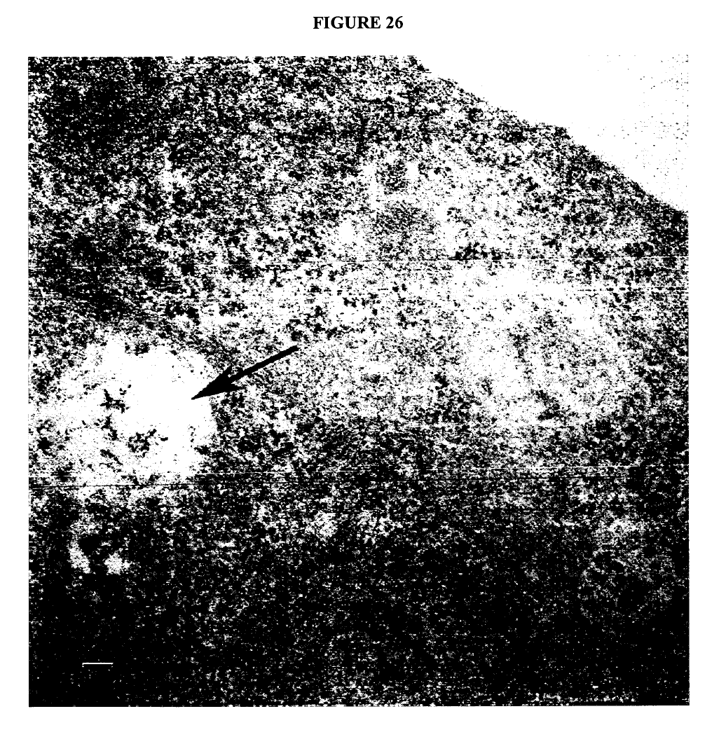

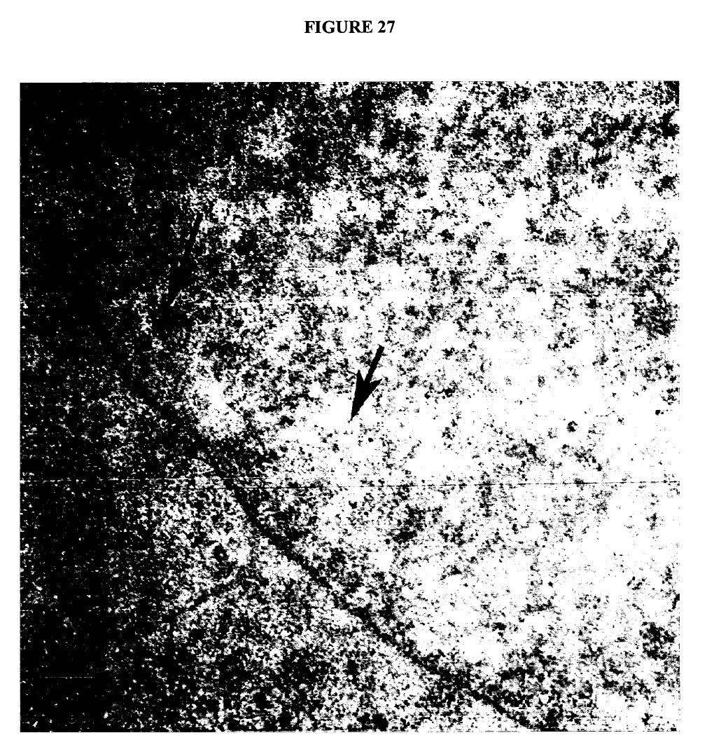

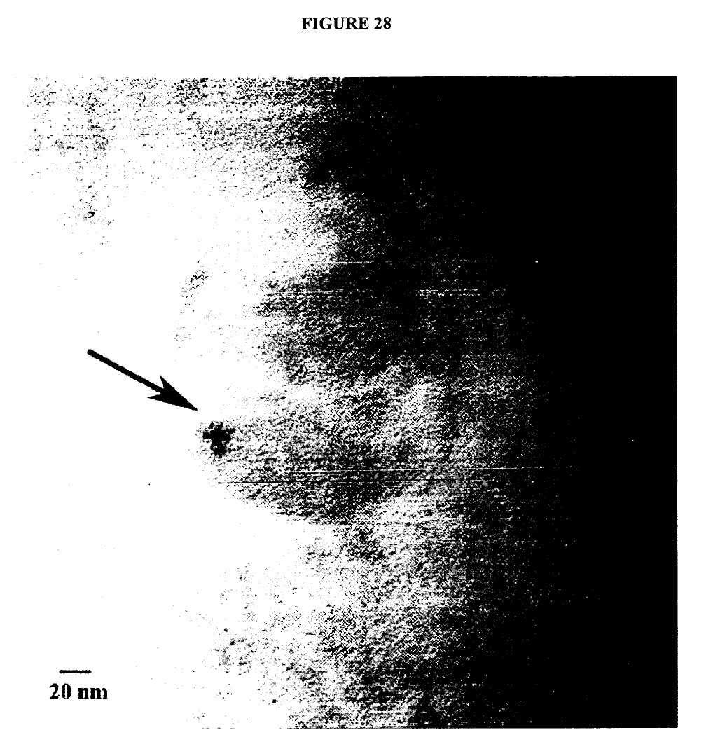

One advantage of using functionalized Au DENPs to target and/or image cancer is the DENPs' ability to differentiate between cancer cells and surrounding non-cancerous cells or tissues by using contrast agents with high electron density. Dendrimers without encapsulated metal nanoparticles cannot achieve this goal (See, e.g., Majoros et al., J. Med. Chem. 2005, 48, 5892; Thomas et al., J. Med. Chem. 2005, 48, 3729). By using TEM imaging techniques, it was possible to clarify the distribution of functionalized Au DENPs in different compartments inside targeted cells. Thus, the present invention provides compositions and methods to characterize mechanisms of targeted drug delivery and therapeutics (e.g., using a DENP comprising a metal nanoparticle of the present invention). Upon 2 h incubation of functionalized Au DENPs with KB cells, the FA-modified {(Au⁰)_{51.2}-G5-FI₅-FA₅-Ac} DENPs were predominantly located in the lysosomes of KB cells with high-level FAR expression (See FIGS. 25a and 25b). A small portion of {(Au⁰)_{51.2}-G5-FI₅-FA₅-Ac} DENPs were also observed in vacuoles and the nucleus (See FIGS. 26 and 27). Uptake of the {(Au⁰)_{51.2}-G5-FI₅-Ac} DENPs lacking FA modification into the lysosomes of the same KB cells was not observed (See FIG. 25c). A very small quantity of {(Au⁰)_{51.2}-G5-FI₅-Ac} DENPs were observed in the vacuoles of some cells (See FIG. 28), that was undetectable using confocal microscopy. Although an understanding of the mechanism is not necessary to practice the present invention and the present invention is not limited to any particular mechanism of action, this uptake may be due to diffusion-driven non-specific binding since control cells not exposed to Au DENPs show no internalized metal nanoparticles. Thus, the present invention provides the high specificity of FA-modified Au DENPs for targeting cells expression FAR.

### Example 5

### One-Step Formation of Functionalized Dendrimer-Stabilized Nanoparticles

### Materials and Methods.

Ethylenediamine core amine-terminated PAMAM dendrimers of generation 5 (G5.NH₂) with a polydispersity index less than 1.08 were purchased from Dendritech (Midland, Mich.). Hydroxyl-terminated G5 dendrimers (G5.NGlyOH) and acetamide-terminated G5 dendrimers (G5.NHAc) were prepared by reacting G5.NH₂ dendrimers with glycidol and acetic anhydride, respectively, and were characterized elsewhere (See Examples 1 and 2, and, e.g., Shi et al., Colloid Surf. A-Physicochem. Eng. Asp. 2006, 272, 139-150; Shi et al., Electrophoresis 2006, 27, 1758-1767). All other chemicals were obtained from Aldrich and used as received. Water was purified using a Milli-Q Plus 185 water purification system (Millipore, Bedford, Mass.) with resistivity higher than 18 MΩ.cm. Regenerated cellulose dialysis membranes (MWCO=10,000) were acquired from Fisher.

Synthesis of Au DSNPs. Synthesis of acetamide-functionalized Au DSNPs. G5.NH₂—Au(III) complexes were first prepared in methanol solution by mixing a methanol solution of HAuCl₄ (3 mL, 1.7 mM) with a methanol solution of the G5.NH₂ dendrimers (3 mL, 0.24 mM) with a molar ratio of G5.NH₂/Au atoms equivalent to 1:7. The yellow HAuCl₄ solution lost its color immediately upon mixing with G5.NH₂ dendrimers, indicating the formation of complexes between the dendrimer terminal amines and the gold anions. The formed complex was denoted as {(Au³⁺)₇-G5.NH₂}. The primary amine groups of {(Au³⁺)₇-G5.NH₂} complexes were acetylated using a described procedure (See, e.g., Majoros et al., Macromolecules 2003, 36, 5526-5529). Briefly, 65 μL of triethylamine was added to the above formed {(Au³⁺)₇-G5.NH₂} solution. A methanolic solution (0.5 mL) of acetic anhydride (48 mg, 500% molar excess of the total primary amines of {(Au³⁺)₇-G5.NH₂}) was added dropwise into the {(Au³⁺)₇-G5.NH₂}/triethylamine mixture solution while it was being stirred vigorously, and the mixture was allowed to react for 24 h. The mixture solution spontaneously changed to pink after 6 h, indicating the formation of Au NPs. The methanol solution of the reaction mixture was extensively dialyzed against PBS buffer (3 times 4 liters) and water (3 times 4 liters) for 3 days to remove the excess of reactants and by-products, followed by lyophilization to get {(Au⁰)₇-G5.NHAc} DSNPs.

Synthesis of hydroxyl-functionalized Au DSNPs. Hydroxyl-functionalized Au DSNPs were prepared by mixing a methanol solution of G5.NGlyOH dendrimers (3 mL, 0.24 mM) with a methanol solution of HAuCl₄ (3 mL, 1.7 mM). The mixture was stirred vigorously for 24 h. After 12 h, the mixture solution changed to a deep-red color, suggesting the spontaneous formation of Au NPs functionalized with hydroxyl groups. The mixture solution was dried under a gentle N₂ stream and then re-dissolved into water, followed by lyophilization. The formed Au NPs was denoted as {(Au⁰)₇-G5.NGlyOH}. Note that the number “7” in the nomenclatures is the number of Au atoms per dendrimer molecule according to the preparation stoichiometry. The denotation of {(Au⁰)₇-G5.NHAc} and {(Au⁰)₇-G5.NGlyOH} does not necessarily represent one single particle.

Characterization. UV-Vis spectra were collected using a Lambda 20 UV-Vis Spectrometer. All Au DSNP and dendrimer-Au (III) complex samples were dissolved in water at the concentration of 3 mg/mL. ¹H NMR spectra of Au DENPs were recorded on a Bruker DRX 500 nuclear magnetic resonance spectrometer. Samples were dissolved in D₂O before NMR measurements. Zeta potential measurements were performed using a Malvern Zetasizer Nano ZS model ZEN3600 (Worcestershire, UK) equipped with a standard 633 nm laser. A JEOL 2010F analytical electron microscope was performed at 200 kV with an energy dispersive spectroscopy (EDS) system attached. A 5-jiL aqueous solution of Au DSNPs (3 mg/mL) was dropped onto a carbon-coated copper grid and air dried before measurements.

Experiments conducted during development of the present invention provide that Au NPs can be spontaneously formed by acetylation of {(Au³⁺)₇-G5.NH₂} complex or by mixing G5.NGlyOH dendrimers with HAuCl₄. In either case, no additional reducing agents were added.

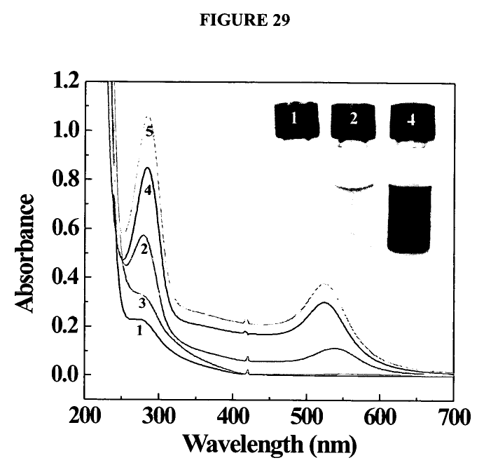

FIG. 29 shows the UV-Vis spectra uf The formed {(Au³⁺)₇-(65.NH₂} complexes (Curve 1), {(Au⁰)₇-G5.NHAc} DSNPs (Curve 2), and {(Au⁰)₇-G5.NGlyOH} DSNPs (Curve 4). The broad shoulder-like absorbance at 280 nm for {(Au³⁺)₇-G5.NH₂} complexes (Curve 1, FIG. 29) is indicative of an ion pair formation between AuCl₄⁻ and G5.NH₂. (See, e.g., Kim et al., Chem. Mater. 2004, 16, 167-172; Esumi et al., Langmuir 2000, 16, 2604-2608). After acetylation of the {(Au³⁺)₇-G5.NH₂} complexes, the formed {(Au⁰)₇-G5.NHAc} DSNPs display a surface plasmon band at 540 nm (Curve 2, FIG. 29), attributed to collective oscillation of free electrons in Au NPs (See, e.g., Alvarez et al., J. Phys. Chem. B 1997, 101, 3706-3712). The {(Au⁰)₇-G5.NGlyOH} DSNPs formed by simply mixing preformed G5.NGlyOH dendrimers with HAuCl₄ display a surface plasmon band at 525 nm (Curve 4, FIG. 29), providing that Au DSNPs with different sizes can be prepared using the two different approaches described above. The size differences of Au NPs were further confirmed by TEM imaging.

Since acetylation of {(Au³⁺)₇-G5.NH₂} complexes spontaneously induced the formation of {(Au⁰)₇-G5.NHAc} DSNPs, it was determined whether {(Au⁰)₇-G5.NGlyOH} DSNPs could be formed by reaction of {(Au³⁺)₇-G5.NH₂} complexes with glycidol molecules. Experiments conducted during development of the present invention showed that this reaction did not induce the formation of the {(Au⁰)₇-G5.NGlyOH} DSNPs, as no characteristic surface plasmon band appeared after the reaction (See Curve 3, FIG. 29). Although an understanding of the mechanism is not necessary to practice the present invention and the present invention is not limited to any particular mechanism of action, in some embodiments, this is due to the natural differences between acetylation and hydroxylation reactions. Although an understanding of the mechanism is not necessary to practice the present invention and the present invention is not limited to any particular mechanism of action, in some embodiments, as compared with a hydroxylation reaction, acetylation is a faster reaction, thereby generating high local energy to assist the reduction of Au (III). The amine groups of G5.NH₂ dendrimers did not readily reduce Au (III) ions to form Au NPs at room temperature, as a slight yellow solution remained for at least three months (See FIG. 29, inset). Although an understanding of the mechanism is not necessary to practice the present invention and the present invention is not limited to any particular mechanism of action, in some embodiments, this is due to the strong complexation between dendrimer terminal amines and AuCl₄⁻ ions, confirmed by NMR studies described below. However, under UV-irradiation (See, e.g., Esumi et al., Langmuir 1998, 14, 3157-3159), thermo treatment (See, e.g., Sun et al., Macromolecular Rapid Communications 2003, 24, 1024-1028), laser irradiation (See, e.g., Hayakawa et al., 2003, 19, 5517-5521), or long-term aging, the amine-terminated dendrimers reduce and stabilize Au NPs. Thus, the present invention provides that acetylation reactions play an important role in the formation of Au DSNPs.

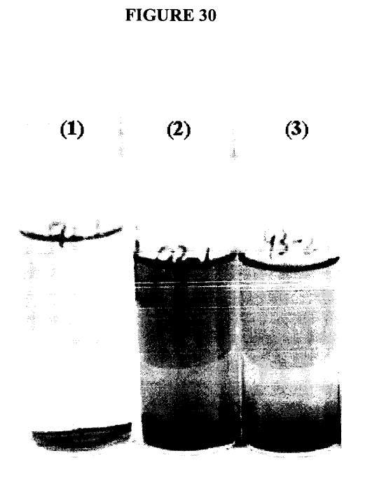

In order to further understand the formation of the synthesized {(Au⁰)₇-G5.NHAc} DSNPs, preformed G5.NHAc dendrimers were attempted to be used as stabilizers. Three experiments were performed in parallel to synthesize acetamide-functionalized Au NPs under similar conditions for use to synthesize {(Au⁰)₇-G5.NHAc} DSNPs (e.g., the molar ratio of dendrimer/Au atom, the amount of triethylamine and/or acetic anhydride added, and the reaction solvent): (1) simply mixing G5.NHAc dendrimers with HAuCl₄; (2) the mixture of G5.NHAc dendrimers and HAuCl₄ added with triethylamine; and (3) the mixture of G5.NHAc dendrimers, HAuCl₄, and triethylamine added with acetic anhydride. In all three cases, black precipitates appeared on the bottom of the vials (See FIG. 30). Thus, the present invention provides that preformed G5.NHAc dendrimers are not able to stabilize Au NPs, while in all cases the G5.NHAc dendrimers with or without triethylamine are able to reduce Au (III) to Au (0). Although an understanding of the mechanism is not necessary to practice the present invention and the present invention is not limited to any particular mechanism of action, in some embodiments, the dendrimer tertiary amines are able to reduce AuCl₄⁻ ions to form Au NPs, while for amine-terminated G5 dendrimers, the strong complexation between the terminal amine groups and AuCl₄⁻ ions impedes the reduction of AuCl₄⁻ ions.

The formation of {(Au⁰)₇-G5.NGlyOH} DSNPs by simply mixing G5.NGlyOH dendrimers with Au (III) ions was quite surprising. Although an understanding of the mechanism is not necessary to practice the present invention and the present invention is not limited to any particular mechanism of action, in some embodiments, the tertiary or secondary terminal amine groups of G5.NGlyOH dendrimers reduce Au (III) and the terminal hydroxyl groups stabilize the formed Au NPs.

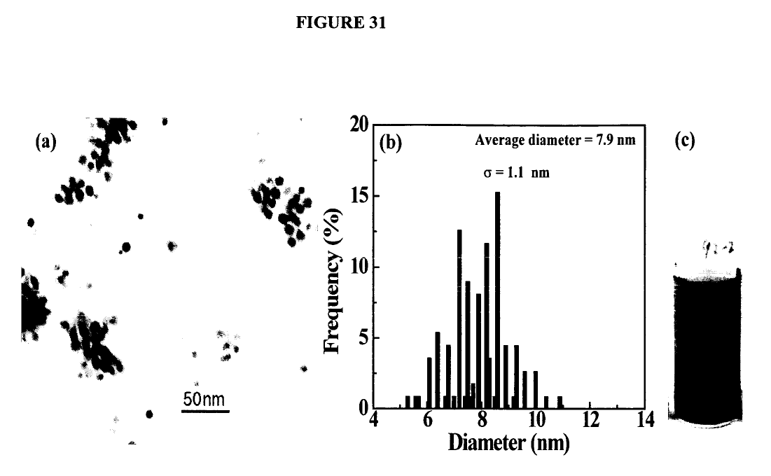

It was also determined that the addition of 5-time molar excess glycidol into G5.NGlyOH dendrimers before mixing with Au (III) ions resulted in the formation of {(Au⁰)₇-G5.NGlyOH} DSNPs (See Curve 5, FIG. 29) with a size similar to that formed in the absence of free glycidol molecules. A TEM image of {(Au⁰)₇-G5.NGlyOH} DSNPs prepared in the presence of free glycidol molecules is shown in FIG. 31. However, simply mixing free glycidol molecules (molar equivalent similar to the terminal groups of G5.NGlyOH dendrimers) with Au (III) ions under similar conditions did not induce the formation of Au NPs. Thus, the present invention provides that both glycidol terminal groups and dendrimer tertiary amine groups are important for the formation of Au NPs.

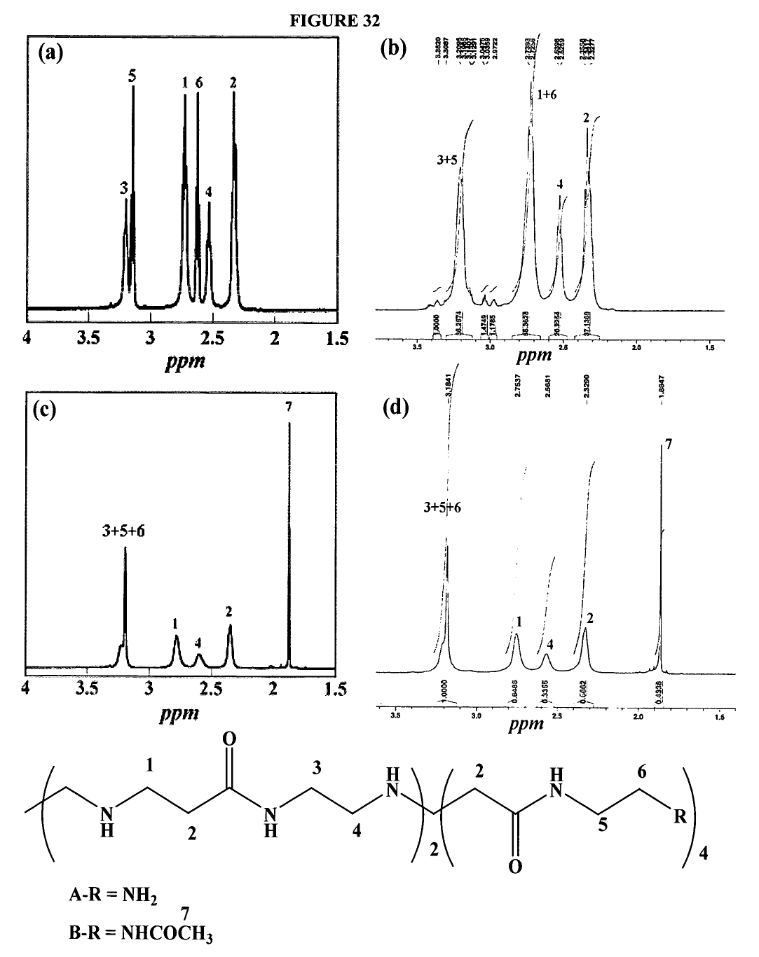

The formation of {(Au³⁺)₇-G5.NH₂} complexes and {(Au⁰)₇-G5.NHAc} DSNPs with acetamide groups was further confirmed by ¹H NMR measurements. FIG. 32 shows the ¹H NMR spectra of G5.NH₂ dendrimers, {(Au³⁺)₇-G5.NH₂} complexes, G5.NHAc dendrimers, and {(Au⁰)₇-G5.NHAc} DSNPs. The chemical shift of protons related to {(Au³⁺)₇-G5.NH₂} (See FIG. 32b) is quite different from those of G5.NH₂ dendrimers (FIG. 32a). Although an understanding of the mechanism is not necessary to practice the present invention and the present invention is not limited to any particular mechanism of action, in some embodiments, this is due to the strong complexation between dendrimer terminal amines and AuCl₄⁻ ions. It is clear that the protons 5 and 6 in G5.NH₂ dendrimers displayed downfield shift and merged with protons 3 and 1, respectively, after the formation of {(Au³⁺)₇-G5.NH₂} complexes. After acetylation of the {(Au³⁺)₇-G5.NH₂} complexes, the formed {(Au⁰)₇-G5.NHAc} DSNPs (See FIG. 32d) exhibited similar ¹H NMR signals to G5.NHAc dendrimers in the absence of Au NPs (See FIG. 32c). This indicates the successful conversion of the amine groups of {(Au³⁺)₇-G5.NH₂} complexes to acetamide groups. The formation of Au DSNPs and the converted dendrimer terminal acetamide groups diminished the strong electrostatic interaction between dendrimer terminal amines and AuCl₄-ions in the original complexes, leading to similar ¹H NMR signals for both G5.NHAc dendrimers and {(Au⁰)₇-G5.NHAc} DSNPs.

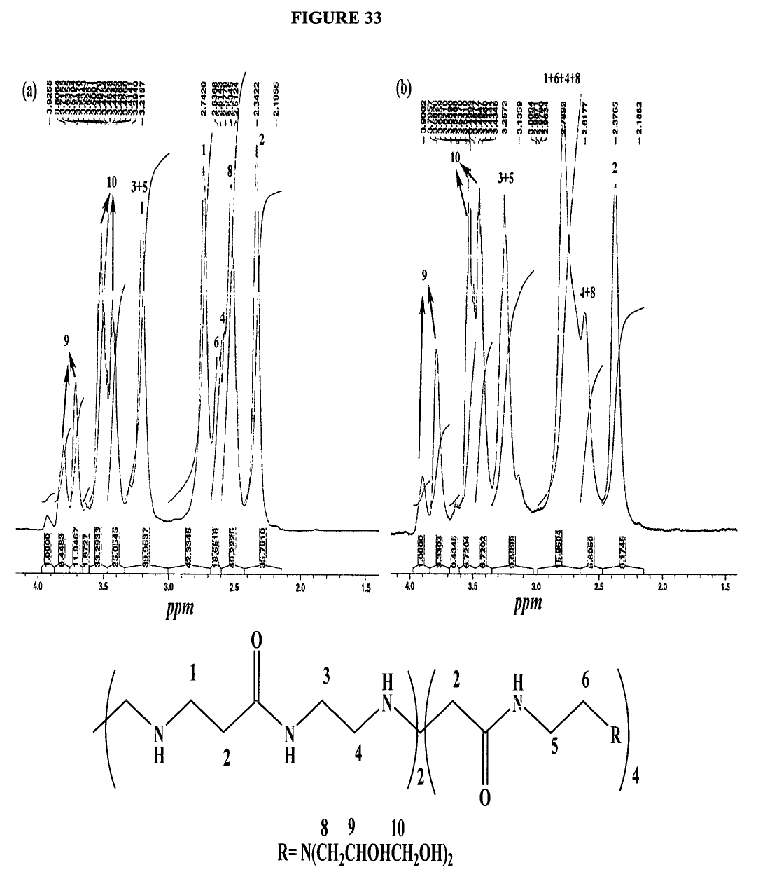

For {(Au⁰)₇-G5.NGlyOH} DSNPs, it is clear that the ¹H NMR signals related to Protons 6, 4, and 8 shifted to downfield as compared with G5.NGlyOH dendrimers (See FIG. 33). Only a part of Protons 4 and 8 still remained in the original position. This implies that after the formation of {(Au⁰)₇-G5.NGlyOH} DSNPs, the secondary terminal amines and tertiary amines of G5.NGlyOH interacted with the surface of Au NPs, while Protons 9 and 10 related to the hydroxyl terminal groups did not show any significant changes as compared with G5.NGlyOH dendrimers. However, the hydroxyl terminal groups helped to stabilize the Au NPs since the terminal acetamide groups of G5.NHAc dendrimers induced the precipitation of Au NPs when directly used to form Au DSNPs as described above.

The morphology of the formed Au DSNPs was observed through TEM imaging.

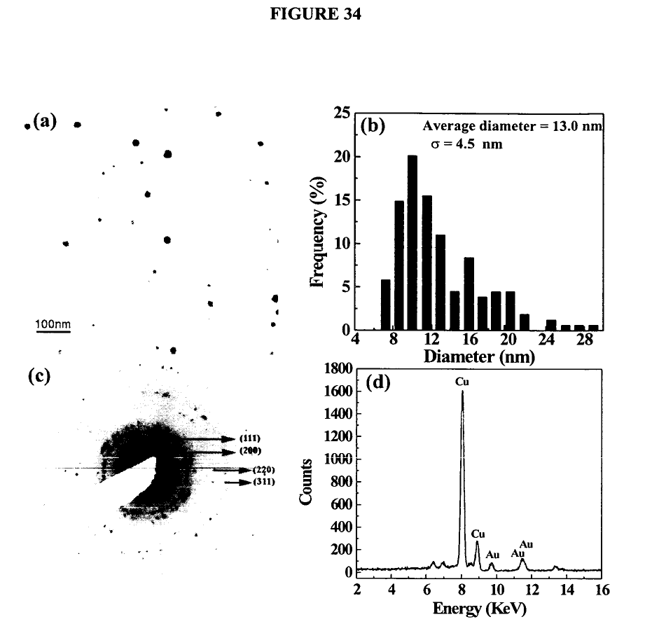

FIGS. 34a and 34b show a bright-field TEM image of {(Au⁰)₇-G5.NHAc} DSNPs and the corresponding size distribution histogram, respectively. The average diameter of {(Au⁰)₇-G5.NHAc} DSNPs is 13±4.5 nm. The {(Au⁰)₇-G5.NHAc} DSNPs are highly crystalline as confirmed by selected area electron diffraction (SAED) pattern (FIG. 34c). The indexed (111), (200), (220), and (311) rings in FIG. 34c are indicatives of the face-centered cubic (fcc) crystal structure of Au DSNPs. An EDS spectrum of the {(Au⁰)₇-G5.NHAc} DSNPs confirms the existence of the Au element (FIG. 34d).

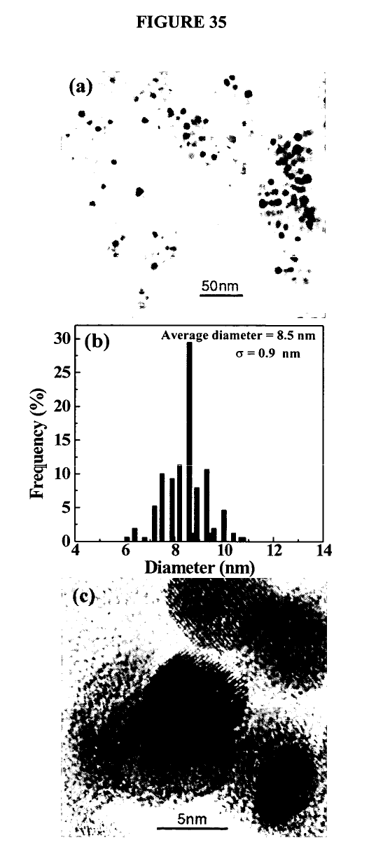

FIG. 35 shows a bright-field TEM image (FIG. 35a), a size distribution histogram (FIG. 35b), and a high-resolution TEM image of the formed {(Au⁰)₇-G5.NGlyOH} DSNPs (FIG. 35c), respectively. The size of the {(Au⁰)₇-G5.NGlyOH} DSNPs (8.5±0.9 nm) was smaller and more monodisperse than that of {(Au⁰)₇-G5.NHAc}. Although an understanding of the mechanism is not necessary to practice the present invention and the present invention is not limited to any particular mechanism of action, in some embodiments, this is due to the existence of the secondary amines and hydroxyl groups of G5.NGlyOH dendrimers that prevent the aggregation of Au NPs during their formation process. A typical EDS spectrum verified the existence of the Au element. A high-resolution TEM image (See FIG. 35c) of the {(Au⁰)₇-G5.NGlyOH} DSNPs shows that most NPs are single crystals, further confirming the crystalline nature of the formed Au DSNPs. Under the TEM conditions, dendrimers on the surfaces of Au NPs are invisible in the TEM images because of their low electron contrast, compared with metal Au.

The formed {(Au⁰)₇-G5.NHAc} and {(Au⁰)₇-G5.NGlyOH} DSNPs are soluble in water and stable after at least 3 months in storage (photographs of the solution of {(Au⁰)₇-G5.NHAc} and {(Au⁰)₇-G5.NGlyOH} DSNPs are shown in the inset of FIG. 29). The surface potential of {(Au³⁺)₇-G5.NH₂} complexes (+39.7 mV) significantly decreased after the formation of {(Au⁰)₇-G5.NHAc} DSNPs (+9.8 mV), confirming the successful transformation of dendrimer terminal amines to acetamide groups. Although an understanding of the mechanism is not necessary to practice the present invention and the present invention is not limited to any particular mechanism of action, in some embodiments, the slight positive charge results from the incomplete acetylation reaction as observed for dendrimers in the absence of metals (See, e.g., Majoros et al., Macromolecules 2003, 36, 5526-5529). For {(Au⁰)₇-G5.NGlyOH} DSNPs, the surface potential was measured to be +26.8 mV. Although an understanding of the mechanism is not necessary to practice the present invention and the present invention is not limited to any particular mechanism of action, in some embodiments, the more positive charge compared to {(Au⁰)₇-G5.NHAc} DSNPs is ascribed to the presence of secondary terminal amines of G5.NGlyOH dendrimers (See, e.g., Shi et al., Colloid Surf. A-Physicochem. Eng. Asp. 2006, 272, 139-150). Thus, the present invention provides that Au DSNPs synthesized using different approaches display different surface charges.

### Example 6

### One-Step Formation and Biofunctionalization of Dendrimer-Stabilized Nanoparticles for Targeted Cancer Therapy

### Materials and Methods

Synthesis and Characterization of Dendrimers Functionalized with Dye and Folate Moieties. Amine-terminated G5 dendrimer (G5.NH₂) was conjugated with FI or both FI and FA moieties, according to described methods (See, e.g., Shi et al., Analyst 2006, 131, 374-381; Majoros et al., J. Med. Chem. 2005, 48, 5892-5899; Quintana et al., Pharm. Res. 2002, 19, 1310-1316). Briefly, G5.NH₂ (60 mg, 0.00225301 mmol) was dissolved in anhydrous DMSO (24 mL). A solution of FI (4.4 mg, 0.00563275 mmol) in DMSO (24 mL) was added dropwise to the above solution under vigorous stirring at room temperature. The reaction was stopped after 24 h. The mixture was dialyzed against PBS buffer and water through a 10,000 MWCO membrane. Lyophilization gave G5.NH₂-FI as an orange solid (60.6 mg, yield 94.0%). For the synthesis of G5.NH₂-FI-FA, FA (3.7 mg, 0.0084004 mmol) and EDC (9.3 mg, 0.021001 mmol) were dissolved in DMSO (3 mL) and the mixture was stirred at room temperature for 3 h. The resulting solution was added dropwise to a solution of G5.NH₂-FI (30 mg, 0.0010501 mmol) in DMSO (12 mL) under vigorous stirring at room temperature. After 3 days, the reaction mixture was dialyzed through a 10,000 MWCO membrane against PBS buffer and then water, followed by lyophilization to give G5.NH₂-FI-FA (31.2 mg, yield 96.4%). The G5.NH₂-FI and G5.NH₂-FI-FA conjugates were characterized by ¹H NMR and matrix-assisted laser desorption ionization-time of flight (MALDI-TOF) mass spectrometry. The numbers of FI and FA moieties conjugated onto each G5 dendrimer were estimated by comparing the differences between the integration values of ¹H NMR signals associated with dendrimers and the FI and FA moieties. The average numbers of FI and FA moieties conjugated onto each G5 dendrimer were estimated to be 4.5 and 4.8, respectively. The molecular weights of G5.NH₂-FI and G5.NH₂-FI-FA conjugates were determined to be 29,900 and 33,600, respectively.

Formation of Au DSNPs Functionalized with Dye and Folate Moieties. G5.NH₂-FI-FA-Au(III) complexes were first prepared by mixing a methanol/water (v/v=1:1) solution of G5.NH₂-FI-FA (6 mL, 0.102 mM) with a methanol solution of HAuCl₄ (108 μL, 39.6 mM) with a molar ratio of G5.NH₂-FI-FA/Au atoms equivalent to 1:7 for 1 h. The formed complex was denoted as {(Au³⁺)₇-G5.NH₂-FI-FA}. The primary amine groups of {(Au³⁺)₇-G5.NH₂-FI-FA} complexes were acetylated (See, e.g., Majoros et al., Macromolecules 2003, 36, 5526-5529). Briefly, 55 μL of triethylamine was added to the above formed {(Au³⁺)₇-G5.NH₂-FI-FA} solution. A methanolic solution (0.5 mL) of acetic anhydride (40 mg, 500% molar excess of the total primary amines of {(Au³⁺)₇-G5.NH₂-FI-FA} was added dropwise into the {(Au³⁺)₇-G5.NH₂-FI-FA}/triethylamine mixture solution while it was being stirred vigorously, and the mixture was allowed to react for 24 h. The reaction mixture solution was extensively dialyzed against PBS buffer (3 times 4 liters) and water (3 times 4 liters) for 3 days to remove the excess of reactants and by-products, followed by lyophilization to get {(Au⁰)₇-G5.NHAc-FI-FA} DSNPs. The control {(Au⁰)₇-G5.NHAc-FI} DSNPs without the presence of FA moieties were prepared in the same manner as the procedure used to prepare {(Au⁰)₇-G5.NTPAc-FI-FA} DSNPs.

Characterization of Biofunctionalized Au DSNPs. UV-Vis spectra were collected using a Lambda 20 UV-Vis Spectrometer. All Au DSNP samples were dissolved in water before UV-Vis measurement. ¹H NMR spectra of Au DSNPs were recorded on a Bruker DRX 500 nuclear magnetic resonance spectrometer. Samples were dissolved in D₂O before NMR measurements. Zeta potential measurements were performed using a Malvern Zetasizer Nano ZS model ZEN3600 (Worcestershire, UK) equipped with a standard 633 nm laser. A JEOL 2010F analytical electron microscope was performed at 200 kV with an energy dispersive spectroscopy (EDS) system attached. A 5-μL aqueous solution of Au DSNPs (3 mg/mL) was dropped onto a carbon-coated copper grid and air dried before measurements.

KB Cell Culture. The KB cells (ATCC, CLL 17, Rockville, Md.) were continuously grown in two 24-well plates, one in FA-free media and the other in regular RPMI 1640 medium (Gibco/BRL, Gaithersburg, Md.) supplemented with penicillin (100 units/mL) (Sigma, St. Louis, Mo.), streptomycin (100 μg/mL) (Sigma, St. Louis, Mo.), 10% heat-inactivated FBS, and 2.5 μM FA. The cells grown in FA-free media express high-level FAR, while the cells grown in FA-containing media express low-level FAR.

Cytotoxicity Analysis. An MTT (3-(4,5-dimethylthiazol-2-yl)-2,5-diphenyltetrazolium bromide) assay was used to quantify the viability of cells. Briefly, 5×10⁴ KB cells per well were seeded into a 96-well plate. After overnight incubation, biofunctionalized Au DSNPs at concentrations ranging from 0 to 2 μM in PBS (pH 7.4) was added. After 24 h incubation with Au DSNPs at 37° C., MTT reagent in PBS was added.

Flow Cytometry. Approximately 2×10⁵ cells per well were seeded in 24-well plates the day before the experiments. An hour before initiating an experiment, the cells were rinsed four times with serum-free and FA-deficient RPMI 1640 media. Functionalized Au DSNPs were added in the final concentrations of 0-300 nM. After 1 h incubation with the functionalized Au DSNTs, KB cells with both high and low-level FAR expression were trypsinized (Gibco/BRL, Gaithersburg, Md.) and suspended in PBS containing 0.1% bovine serum albumin (Sigma, St. Louis, Mo.) and analyzed using a Becton Dickinson FACScan analyzer. The FL1-fluorescence of 10,000 cells was measured, and the mean fluorescence of gated viable cells was quantified.

Confocal Laser Scanning Microscopy. Confocal microscopic analysis was performed in cells plated on a plastic cover-slip using a Zeiss LSM 510 confocal microscope (Thornwood, N.Y.). FI fluorescence was excited with a 488-nm argon blue laser and emission was measured through a 505-525 barrier filter. The optical section thickness was set at 5 μm. The cells were incubated with functionalized Au DSNPs (25 nM) for 2 h, followed by rinsing with PBS buffer. The nuclei were counterstained with 1 μg/mL of Hoescht33342, using a standard procedure. Samples were scanned using a 60× water immersion objective lens and magnified with FluoView software.

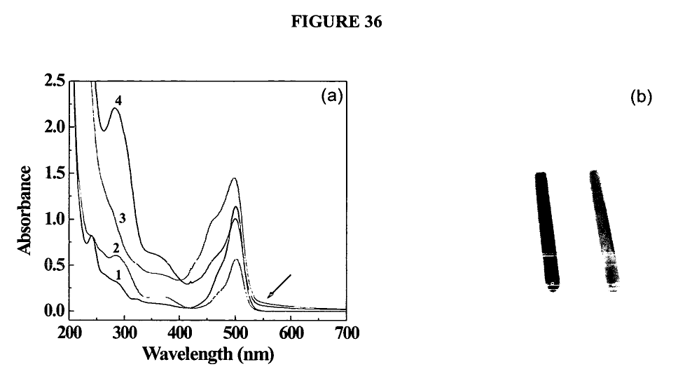

UV-Vis spectra of G5.NHAc-FI and G5.NHAc-FI-FA dendrimers (Curve 1 and Curve 2, respectively), and {(Au⁰)₇-G5.NHAc-FI} and {(Au⁰)₇-G5.NHAc-FI-FA} DSNPs (Curve 3 and Curve 4, respectively) are shown in FIG. 36. The arrow shows the shoulder, indicating The formation of Au DSNPs is indicated by the shoulder shape of {(Au⁰)₇-G5.NHAc-FI} and {(Au⁰)₇-G5.NHAc-FI-FA} DSNPs (See Arrow in FIG. 36). The red color of the Au DSNP solution in FIG. 36b confirms the formation of Au DSNPs.

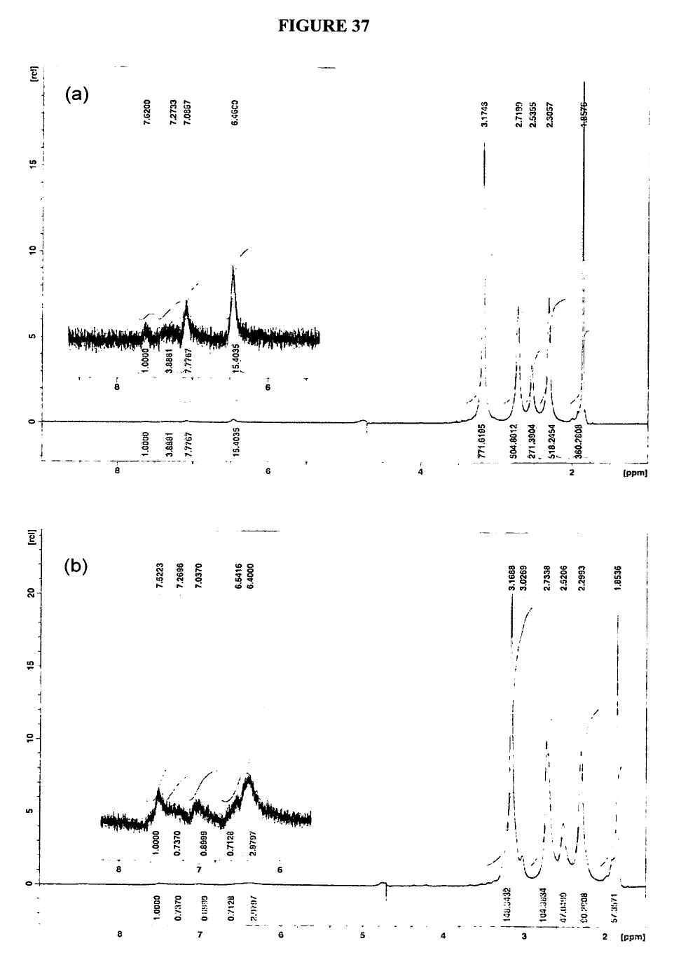

¹H NMR was used to characterize the formation of dendrimer complexes. ¹H NMR verified the successful acetylation of {(Au³⁺)₇-G5.NH₂-FI} and {(Au³⁺)₇-G5.NH₂-FI-FA} complexes (See FIG. 37). Zeta potential data of {(Au⁰)₇-G5.NHAc-FI} (+9.2 mV) and {(Au⁰)₇-G5.NHAc-FI-FA} (+5.3 mV) DSNPs confirmed the transformation of the remaining amine groups of {(Au³⁺)₇-G5.NH₂-FI} and {(Au³⁺)₇-G5.NH₂-FI-FA} complexes to acetamide groups (See FIG. 37).

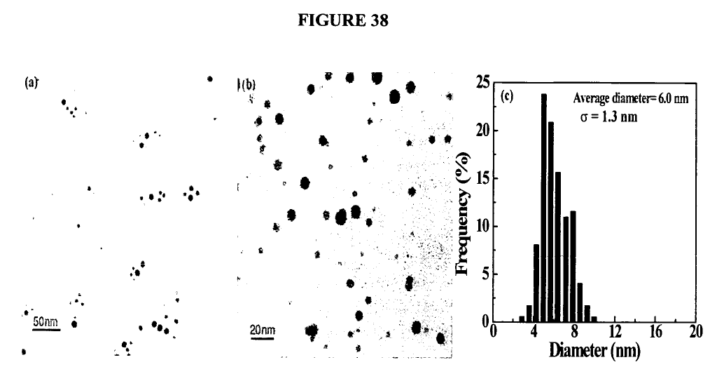

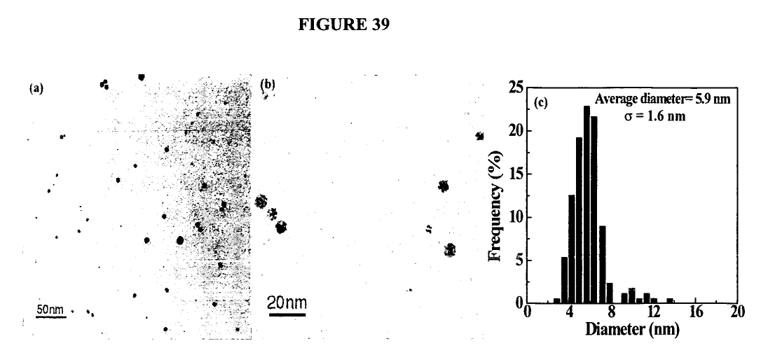

TEM images were utilized to characterize the complexes. The images showed that the formed biofunctionalized Au DSNPs are relatively monodisperse and have a narrow size distribution. Large scale and magnified TEM images of {(Au⁰)₇-G5.NHAc-Fl} and {(Au⁰)₇-G5.NHAc-FI-FA} DSNPs are shown in FIGS. 38 and 39, respectively.

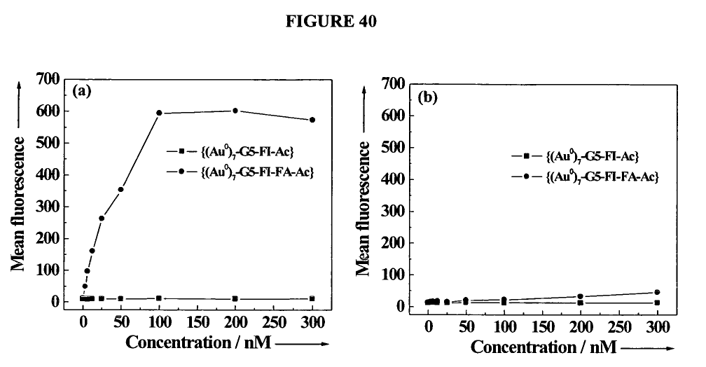

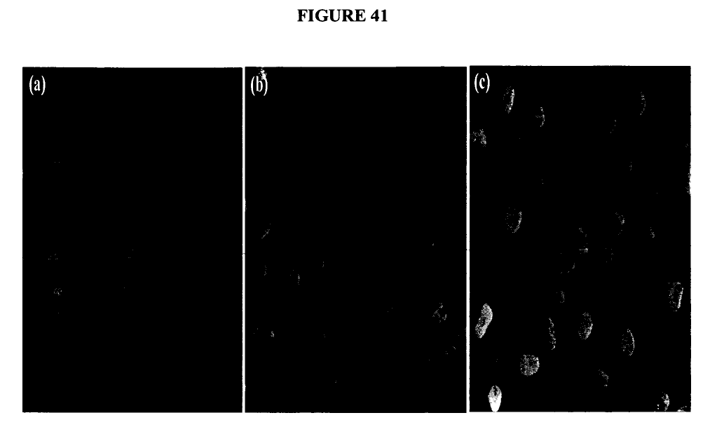

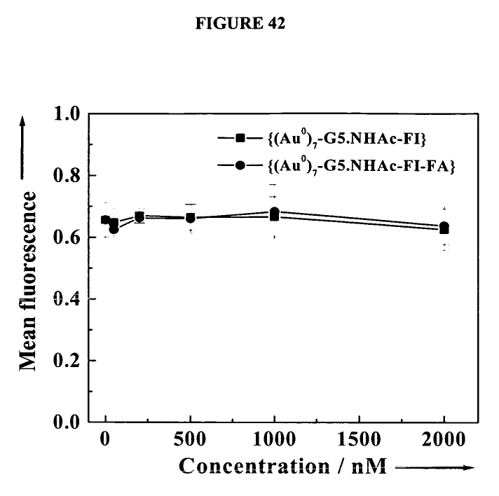

The ability of the DSNPs to target and bind to target cells was determined. Various KB cells, some expressing high levels of folate receptor (FAR) whereas others expressed low levels of FAR, were used in experiments. Flow cytometric analysis showed that only {(Au⁰)₇-G5.NHAc-FI-FA} DSNPs functionalized with folate moieties were able to specifically bind KB cells expressing high-level folate receptor (See FIG. 40). Furthermore, only FA-functionalized Au DSNPs were taken-up by KIB cells with high-level FAR whereas DSNPs comprising dendrimers lacking FA were not found within the KB cells (See FIG. 41). The potential toxicity of the DSNPs to target cells was also determined. MTT data showed that the biofunctionalized Au DSNPs were non-toxic at concentrations up to 2 μM (See FIG. 42).

All publications and patents mentioned in the above specification are herein incorporated by reference. Various modifications and variations of the dcscribed method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the present invention.

We claim:

1. A composition comprising a functionalized dendrimer nanoparticle.

2. The composition of claim 1, wherein said nanoparticle is a metal.

3. The composition of claim 2, wherein said metal is gold.

4. The composition of claim 2, wherein said metal is selected from the group consisting of copper, platinum, silver, lead, cobalt, iron, manganese, chromium and nickel.

5. The composition of claim 1, wherein said functionalized dendrimer nanoparticle is selected from the group consisting of a hydroxyl-functionalized dendrimer encapsulated nanoparticle, an acetamide-functionalized dendrimer encapsulated nanoparticle, an acetamide-functionalized dendrimer stabilized nanoparticle, and a hydroxyl-functionalized dendrimer stabilized nanoparticle.

6. The composition of claim 5, wherein said hydroxyl-functionalized dendrimer encapsulated nanoparticle is synthesized by providing a dendrimer encapsulated nanoparticle and reacting said dendrimer encapsulated nanoparticle with glycidol.

7. The composition of claim 5, wherein said acetamide-functionalized dendrimer encapsulated nanoparticle is synthesized by providing a dendrimer encapsulated nanoparticle and reacting said dendrimer encapsulated nanoparticle with acetic anhydride.

8. The composition of claim 5, wherein synthesizing said acetamide-functionalized dendrimer stabilized nanoparticle comprises:
a) providing:
i) a solution comprising {(Au³⁺)₇-G5.NH₂} complex and triethylamine; and
ii) a solution of acetic anhydride; and
b) mixing the two solutions.

9. The composition of claim 5, wherein synthesizing said hydroxyl-functionalized dendrimer stabilized nanoparticle comprises:
a) providing:
i) a solution comprising G5.NGlyOH dendrimers
ii) a solution comprising HAuCl₄; and
b) mixing the two solutions.

10. The composition of claim 1, wherein said functionalized dendrimer nanoparticle comprises one or more functional groups.

11. The composition of claim 10, wherein said functional group is selected from the group consisting of a therapeutic agent, a targeting agent, an imaging agent, and a biological monitoring agent.

12. The composition of claim 11, wherein said targeting agent comprises folic acid.

13. The composition of claim 11, wherein said imaging agent comprises fluorescein isothiocyanate.

14. The composition of claim 11, wherein said therapeutic agent is selected from the group consisting of a chemotherapeutic agent, an anti-oncogenic agent, an anti-vascularizing agent, a tumor suppressor agent, an anti-microbial agent, and an expression construct comprising a nucleic acid encoding a therapeutic protein.

15. A method of targeting a functionalized dendrimer nanoparticle to a cell comprising:
a) providing:
i) a functionalized dendrimer nanoparticle, wherein said functionalized dendrimer nanoparticle comprises a cell specific targeting moiety; and
ii) a cell; and
b) exposing said cell to said functionalized dendrimer nanoparticle under conditions such that said targeting moiety interacts with said cell.

16. The method of claim 15, wherein said functionalized dendrimer nanoparticle is selected from the group consisting of a hydroxyl-functionalized dendrimer encapsulated nanoparticle, an acetamide-functionalized dendrimer encapsulated nanoparticle, an acetamide-functionalized dendrimer stabilized nanoparticle, and a hydroxyl-functionalized dendrimer stabilized nanoparticle.

17. The method of claim 15, wherein said cell is a cancer cell.

18. The method of claim 15, wherein said cell specific targeting moiety comprises folic acid.

19. The method of claim 15, wherein said functionalized dendrimer nanoparticle is internalized by said cell.

20. The method of claim 15, wherein said functionalized dendrimer nanoparticle further comprises one or more functional groups, wherein said one or more functional groups are selected from the group consisting of a therapeutic agent, a targeting agent, an imaging agent, and a biological monitoring agent.
